(19) United States
(12) Patent Application Publication
(10) Pub. No.: US 2009/0099168 A1
(43) Pub. Date: Apr. 16, 2009

(54) HIV Integrase inhibitors

(76) Inventors: Monica Donghi, Pomezia (IT); Cristina Gardelli, Pomezia (IT)

Correspondence Address: MERCK AND CO., INC, P O BOX 2000, RAHWAY, NJ, 07065-0907, US

(21) Appl. No.: 12/316,027

(22) Filed: Dec. 9, 2008

Related U.S. Application Data
(62) Division of application No. 10/587,601, filed on Jul. 28, 2006.
(60) Provisional application No. 60/551,601, filed on Mar. 9, 2004.

(51) Int. Cl. A61K 31/5377; C07D 471/16; A61P 31/18; A61K 31/4985

(52) U.S. Cl. 514/233.2; 544/250; 514/250; 544/115

(57) ABSTRACT

Pyridopyrazine- and pyrimidopyrazine-dione compounds are inhibitors of HIV integrase and inhibitors of HIV replication. In one embodiment, the dihydroxypyridine carboxamides are of Formula I:

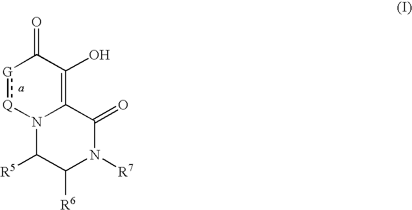

wherein G, Q, bond a, R⁵, R⁶ and R⁷ are defined herein. The compounds are useful in the prevention and treatment of infection by HIV and in the prevention, delay in the onset, and treatment of AIDS. The compounds are employed against HIV infection and AIDS as compounds per se or in the form of pharmaceutically acceptable salts. The compounds and their salts can be employed as ingredients in pharmaceutical compositions, optionally in combination with other antivirals, immunomodulators, antibiotics or vaccines.

### FIELD OF THE INVENTION

The present invention is directed to pyridopyrazine- and pyrimidopyrazine-dione compounds and pharmaceutically acceptable salts thereof, their synthesis, and their use as inhibitors of the HIV integrase enzyme. The compounds and pharmaceutically acceptable salts thereof of the present invention are useful for preventing or treating infection by HIV and for preventing, treating or delaying the onset of AIDS.

### BACKGROUND OF THE INVENTION

A retrovirus designated human immunodeficiency virus (HIV) is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the insertion by virally-encoded integrase of proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277 (1985)]. Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells. The particular advantage of the present invention is highly specific inhibition of HIV integrase and HIV replication.

The following references are of interest as background:

U.S. Pat. No. 6,380,249, U.S. Pat. No. 6,306,891, and U.S. Pat. No. 6,262,055 disclose 2,4-dioxobutyric acids and acid esters useful as HIV integrase inhibitors.

WO 01/00578 discloses 1-(aromatic- or heteroaromatic-substituted)-3-(heteroaromatic substituted)-1,3-propanediones useful as HIV integrase inhibitors.

US 2003/0055071 (corresponding to WO 02/30930), WO 02/30426, and WO 02/55079 each disclose certain 8-hydroxy-1,6-naphthyridine-7-carboxamides as HIV integrase inhibitors.

WO 02/036734 discloses certain aza- and polyaza-naphthalenyl ketones to be HIV integrase inhibitors.

WO 03/016275 discloses certain compounds having integrase inhibitory activity.

WO 03/35076 discloses certain 5,6-dihydroxypyrimidine-4-carboxamides as HIV integrase inhibitors, and WO 03/35077 discloses certain N-substituted 5-hydroxy-6-oxo-1,6-dihydropyrimidine-4-carboxamides as HIV integrase inhibitors.

WO 03/062204 discloses certain hydroxynaphthyridinone carboxamides that are useful as HIV integrase inhibitors.

WO 04/004657 discloses certain hydroxypyrrole derivatives that are HIV integrase inhibitors.

### SUMMARY OF THE INVENTION

The present invention is directed to pyridopyrazine- and pyrimidopyrazine-dione compounds. These compounds are useful in the inhibition of HIV integrase, the prevention of infection by HIV, the treatment of infection by HIV and in the prevention, treatment, and delay in the onset of AIDS and/or ARC, either as compounds or their pharmaceutically acceptable salts or hydrates (when appropriate), or as pharmaceutical composition ingredients, whether or not in combination with other HIV/AIDS antivirals, anti-infectives, immunomodulators, antibiotics or vaccines. More particularly, the present invention includes compounds of Formula I, and pharmaceutically acceptable salts thereof:

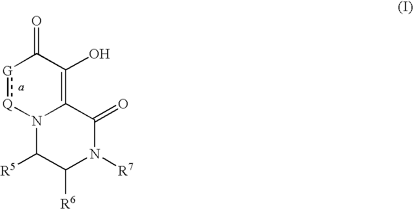

wherein:

### G is C—R¹, CH—R¹, N, or N—R²;

Q is C—R³, C—R⁴, CH—R³ or CH—R⁴, with the proviso that (i) when G is C—R¹, then Q is C—R³, (ii) when G is CH—R¹, then Q is CH—R³, (iii) when G is N, then Q is C—R⁴, and (iv) when G is N—R², then Q is CH—R⁴;
bond “a” is a single bond or a double bond between G and Q, with the proviso that (i) when G is N or C—R¹, bond “a” is a double bond, and (ii) when G is CH—R¹ or N—R², bond “a” is a single bond;

### R¹ is:

(1) H,

(2) halogen,

(3) C₁₋₆ alkyl,

(4) C₁₋₆alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₆ alkylene-O—C₁₋₆ alkyl,
(e) —N(R^{a})—C(═O)—C(═O)—N(R^{a})R^{b},
(f) —OH,
(g) —HetD, or
(h) —N(R^{a})—C₁₋₆ alkylene-HetA,

(5) HetA,

(6) C(═O)—R^{a},

(7) C(═O)-aryl, or

(8) C(═O) —HetA;

R² is H or C₁₋₆ alkyl;

### R³ is:

(1) H,

(2) C₁₋₆ alkyl,

(3) C₁₋₆ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₆ alkylene-O—C₁₋₆ alkyl,
(e) —N(R^{a})—C(═O)—C(═O)—N(R^{a})R^{b},
(f) —HetD,
(g) —N(R^{a})—C₁₋₆ alkylene-HetA, or

(4) C(═O)—C₁₋₆ alkyl,

(5) CO₂H,

(6) C(═O)—O—C₁₋₆ alkyl,

(7) C(═O)N(R^{a})R^{b}, or

(8) C(═O) —HetF;

### R⁴ is:

(1) H,

(2) C₁₋₆ alkyl, or

(3) C₁₋₆ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₆ alkylene-O—C₁₋₆ alkyl,
(e) —N(R^{a})—C(═O)—C(═O)—N(R^{a})R^{b},
(f) —HetD, or
(g) —N(R^{a})—C₁₋₆ alkylene-HetA;

### R⁵ is:

(1) H,

(2) C₁₋₆ alkyl, or

(3) C₁₋₆ alkyl substituted with:
(a) —CO₂H,
(b) —C(═O)—O—C₁₋₆ alkyl,
(c) —C(═O)—C₁₋₆ alkyl,
(d) —N(R^{a})R^{b},
(e) —C(═O)N(R^{a})R^{b},
(f) —N(R^{a})—C(═O)—R^{b},
(g) —N(R^{a})—SO₂R^{b},
(h) —N(R^{a})—C(═O)—C(═O)—N(R^{a})R^{b},
(i) —HetF,
(j) —C(═O)—HetF, or
(k) —N(R^{a})—C(═O)—C(═O)—HetF;
   or alternatively R⁴ and R⁵ together with the carbon atoms to which each is attached and the fused ring N atom therebetween form a ring such that the compound of Formula I is a compound of Formula Ia or Ib:

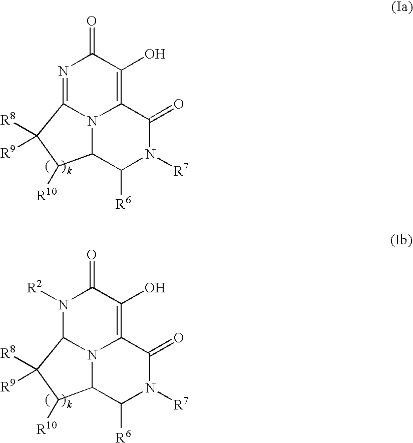

wherein k is an integer equal to 1 or 2;
R⁶ is H or C₁₋₆ alkyl;
R⁷ is C₁₋₆ alkyl substituted with T, wherein T is:
(A) aryl or aryl fused to a 5- or 6-membered heteroaromatic ring containing from 1 to 4 heteroatoms independently selected from N, O and S, wherein the aryl or fused aryl is optionally substituted with from 1 to 5 substituents each of which is independently:
   (1) —C₁₋₆ alkyl optionally substituted with —OH, —O—C₁₋₆ alkyl, —O—C₁₋₆ haloalkyl, —CN, —NO₂, —N(R^{a})R^{b}, —C(═O)N(R^{a})R^{b}, —C(═O)R^{a}, —CO₂R^{a}, —S(O)ₙR^{a} where n is an integer equal to zero or 1 or 2, —SO₂N(R^{a})R^{b}, —N(R^{a})C(═O)R^{b}, —N(R^{a})CO₂R^{b}, —N(R^{a})SO₂R^{b}, —N(R^{a})SO₂N(R^{a})R^{b}, —OC(═O)N(R^{a})R^{b}, or —N(R^{a})C(═O)N(R^{a})R^{b},
   (2) —O—C₁₋₆ alkyl,
   (3) —C₁₋₆ haloalkyl,
   (4) —O—C₁₋₆ haloalkyl,
   (5) —OH,
   (6) halo,
   (7) —CN,
   (8) —NO₂,
   (9) —N(R^{a})R^{b},
   (10) —C(═O)N(R^{a})R^{b},
   (11) —C(═O)R^{a},
   (12) —CO₂R^{a},
   (13) —SR^{a},
   (14) —S(═O)R^{a},
   (15) —SO₂R^{a},
   (16) —SO₂N(R^{a})R^{b},
   (17) —N(R^{a})SO₂R^{b},
   (18) —N(R^{a})SO₂N(R^{a})R^{b},
   (19) —N(R^{a})C(═O)R^{b},
   (20) —N(R^{a})C(═O)—C(═O)N(R^{a})R^{b},
   (21) —N(R^{a})CO₂R^{b},
   (22) phenyl,
   (23) benzyl,
   (24) —HetB,
   (25) —C(═O)—HetB, or
   (26) —HetC, or
(B) a 5- or 6-membered heteroaromatic ring containing from 1 to 4 heteroatoms independently selected from N, O and S; wherein the heteroaromatic ring is
   (i) optionally substituted with from 1 to 4 substituents each of which is independently halogen, —C₁₋₆ alkyl, —C₁₋₆ haloalkyl, —O—C₁₋₆ alkyl, —O—C₁₋₆ haloalkyl, or hydroxy; and
   (ii) optionally substituted with 1 or 2 substituents each of which is independently aryl or —C₁₋₆ alkyl substituted with aryl;

### R⁸ is:

(1) H,

(2) C₁₋₆ alkyl,

(3) N(R^{a})R^{b},

(4) N(R^{a})—CO₂R^{b},

(5) N(R^{a})—SO₂R^{b},

(6) N(R^{a})—C(═O)—R^{b},

(7) N(R^{a})—C(═O)—N(R^{a})R^{b},

(8) N(R^{a})—C(═O)—C(═O)—N(R^{a})R^{b},

(9) HetF,

(10) N(R^{a})—C(═O)—HetF, or

(11) N(R^{a})—C(═O)—C(═O)—HetF;

R⁹ is H, C₁₋₆ alkyl, or C₁₋₆ alkyl substituted with U, wherein U independently has the same definition as T;
each R¹⁰ is independently H or C₁₋₆ alkyl;
each HetA is independently:
(A) a 5- or 6-membered heteroaromatic ring containing from 1 to 4 heteroatoms independently selected from N, O and S; wherein the heteroaromatic ring is attached to the rest of the compound via a carbon atom in the ring, and wherein the heteroaromatic ring is:
   (i) optionally substituted with 1 or 2 substituents each of which is independently a —C₁₋₄ alkyl; and
   (ii) optionally substituted with aryl or —C₁₋₄ alkylene-aryl; or
(B) a 9- or 10-membered aromatic heterobicyclic fused ring system containing from 1 to 4 heteroatoms independently selected from N, O and S; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
   (i) optionally substituted with 1 or 2 substituents each of which is independently a —C₁₋₄ alkyl; and
   (ii) optionally substituted with aryl or —C₁₋₄ alkylene-aryl;
      each HetB is independently a C₄₋₇ azacycloalkyl or a C₃₋₆ diazacycloalkyl, either of which is optionally substituted with from 1 to 4 substituents each of which is oxo or C₁₋₆ alkyl;
      each HetC is independently a 5- or 6-membered heteroaromatic ring containing from 1 to 4 heteroatoms independently selected from N, O and S, wherein the heteroaromatic ring is optionally substituted with from 1 to 4 substituents each of which is independently halo, —C₁₋₆ alkyl, —C₁₋₆ haloalkyl, —O—C₁₋₆ alkyl, —O—C₁₋₆ haloalkyl, or hydroxy; or
      each HetD is independently a 4- to 7-membered saturated heterocyclic ring containing at least one carbon atom and a total of from 1 to 4 heteroatoms independently selected from 1 to 4 N atoms, from 0 to 2 O atoms, and from 0 to 2 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with 1 or 2 substituents each of which is independently a —C₁₋₄ alkyl, —C₁₋₄ alkylene-N(R^{a})R^{b}, or —C(═O)OR^{a}; and
(ii) optionally substituted with aryl, —C₁₋₄ alkylene-aryl, HetE, or —C₁₋₄ alkylene-HetE; wherein HetE is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S;
   each HetF is independently a 4- to 7-membered saturated heterocyclic ring containing 1 or 2 N atoms, zero or 1 O atom, and zero or 1 S atom, wherein any ring S atom is optionally oxidized to SO or SO₂, and wherein the heterocyclic ring is attached to the rest of the compound via a N atom in the ring, and wherein the heterocyclic ring is optionally substituted with 1 or 2 substituents each of which is independently a —C₁₋₆ alkyl;
   each aryl is independently phenyl or naphthyl;
   each R^{a} is independently H or C₁₋₆ alkyl; and
   each R^{b} is independently H or C₁₋₆ alkyl.

The present invention also includes pharmaceutical compositions containing a compound of the present invention and methods of preparing such pharmaceutical compositions. The present invention further includes methods of treating AIDS, methods of delaying the onset of AIDS, methods of preventing AIDS, methods of preventing infection by HIV, and methods of treating infection by HIV.

Other embodiments, aspects and features of the present invention are either further described in or will be apparent from the ensuing description, examples and appended claims.

### DETAILED DESCRIPTION OF THE INVENTION

The present invention includes compounds of Formula I above, and pharmaceutically acceptable salts thereof. These compounds and pharmaceutically acceptable salts thereof are HIV integrase inhibitors.

A first embodiment of the present invention is a compound of Formula I, or a pharmaceutically acceptable salt thereof, wherein

### R³ is:

(1) H,

(2) C₁₋₆ alkyl,

(3) C₁₋₆ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₆ alkylene-O—C₁₋₆ alkyl,
(e) —N(R^{a})—C(═O)—C(═O)—N(R^{a})R^{b},
(f) —HetD, or
(g) —N(R^{a})—C₁₋₆ alkylene-HetA, or

(4) C(═O)—C₁₋₆ alkyl;

### R⁴ is:

(1) H,

(2) C₁₋₆ alkyl, or

(3) C₁₋₆ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₆ alkylene-O—C₁₋₆ alkyl,
(e) —N(R^{a})—C(═O)—C(═O)—N(R^{a})R^{b},
(f) —HetD, or
(g) —N(R^{a})—C₁₋₆ alkylene-HetA; and
   R⁵ and R⁶ are each independently H or C₁₋₆ alkyl; and all other variables are as originally defined (i.e., as defined in the Summary of the Invention).

A second embodiment of the present invention is a compound of Formula I, or a pharmaceutically acceptable salt thereof, wherein R¹ is:

(1) H,

(2) halogen,

(3) C₁₋₄ alkyl,

(4) C₁₋₄ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₃ alkylene-O—C₁₋₄ alkyl (e.g., —N(R^{a})—C₂₋₃ alkylene-O—C₁₋₄ alkyl),
(e) —N(R^{a})—C(═O)—C(═O)—N(R^{a})R^{b},
(f) —OH,
(g) —HetD, or
(h) —N(R^{a})—C₁₋₃ alkylene-HetA,

(5) HetA,

(6) C(═O)—R^{a},

(7) C(═O)-aryl, or

(8) C(═O)—HetA;

and all other variables are as originally defined or as defined in the first embodiment.

A third embodiment of the present invention is a compound of Formula I, or a pharmaceutically acceptable salt thereof, wherein R¹ is:

(1) H,

(2) halogen,

(3) C₁₋₃ alkyl,

(4) C₁₋₃ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₃ alkylene-O—C₁₋₃ alkyl (e.g., —N(R^{a})—C₂₋₃ alkylene-O—C₁₋₄ alkyl),
(e) —N(R^{a})—C(═O)—C(═O)—N(R^{a})R^{b},
(f) —OH,
(g) —HetD, or
(h) —N(R^{a})—C₁₋₃ alkylene-HetA,

(5) HetA,

(6) C(═O)—R^{a},

(7) C(═O)-aryl, or

(8) C(═O)—HetA;

and all other variables are as originally defined or as defined in the first embodiment.

A fourth embodiment of the present invention is a compound of Formula I, or a pharmaceutically acceptable salt thereof, wherein R¹ is:
(1) H,
(2) C₁₋₃ alkyl,
(3) chloro,
(4) bromo,
(5) CH₂—N(R^{a})R^{b},
(6) CH(CH₃)—N(R^{a})R^{b},
(7) CH₂—N(R^{a})—C(═O)—R^{b},
(8) CH(CH₃)—N(R^{a})—C(═O)—R^{b},
(9) CH₂—N(R^{a})—SO₂R^{b},
(10) CH(CH₃)—N(R^{a})—SO₂R^{b},
(11) CH₂—N(R^{a})—C₁₋₃ alkylene-O—C₁₋₁₃ alkyl (e.g., CH₂—N(R^{a})—C₂₋₃ alkylene-O—C₁₋₁₃ alkyl),
(12) CH(CH₃)—N(R^{a})—C₁₋₃ alkylene-O—C₁₋₃ alkyl (e.g., CH(CH₃)—N(R^{a})—C₂₋₃ alkylene-O—C₁₋₃ alkyl),
(13) CH₂—N(R^{a})—C(═O)—C(═O)—N(R^{a})R^{b},
(14) CH(CH₃)—N(R^{a})—C(═O)—C(═O)—N(R^{a})R^{b},
(15) CH₂OH,
(16) CH(CH₃)OH,
(17) CH₂—HetD,
(18) CH(CH₃)—HetD,
(19) CH₂—N(R^{a})—CH₂—HetA,
(20) CH(CH₃)—N(R^{a})—CH₂—HetA,
(21) HetA, or
(22) C(═O)—R^{a};
   and all other variables are as originally defined or as defined in the first embodiment.

A fifth embodiment of the present invention is a compound of Formula I, or a pharmaceutically acceptable salt thereof, wherein R¹ is:

(1) H,

(2) CH₃,

(3) chloro,

(4) bromo,

(5) CH₂—NH(CH₃),

(6) CH₂—N(CH₃)₂,

(7) CH(CH₃)—NH(CH₃),

(8) CH(CH₃)—N(CH₃)₂,

(9) CH(CH₃)—NH(CH(CH₃)₂),

(10) CH₂—NH—C(═O)CH₃,

(11) CH₂—N(CH₃)—C(═O)CH₃,

(12) CH(CH₃)—NH—C(═O)CH₃,

(13) CH(CH₃)—N(CH₃)—C(═O)CH₃,

(14) CH₂—NH—SO₂CH₃,

(15) CH₂—N(CH₃)—SO₂CH₃,

(16) CH(CH₃)—NH—SO₂CH₃,

(17) CH(CH₃)—N(CH₃)—SO₂CH₃,

(18) CH₂—NH—(CH₂)₁₋₂—OCH₃ (e.g., CH₂—NH—(CH₂)₂—OCH₃),

(19) CH₂—N(CH₃)—(CH₂)₁₋₂—OCH₃ (e.g., CH₂—N(CH₃)—(CH₂)₂—OCH₃),

(20) CH(CH₃)—NH—(CH₂)₁₋₂—OCH₃ (e.g., CH(CH₃)—NH—(CH₂)₂—OCH₃),

(21) CH(CH₃)—N(CH₃)—(CH₂)₁₋₂—OCH₃ (e.g., CH(CH₃)—N(CH₃)—(CH₂)₂—OCH₃),

(22) CH₂—NH—C(═O)—C(═O)—N(CH₃)₂,

(23) CH₂—N(CH₃)—C(═O)—C(═O)—N(CH₃)₂,

(24) CH(CH₃)—NH—C(═O)—C(═O)—N(CH₃)₂,

(25) CH(CH₃)—N(CH₃)—C(═O)—C(═O)—N(CH₃)₂,

(26) CH₂OH,

(27) CH(CH₃)OH,

(28) CH₂—HetD,

(29) CH(CH₃)—HetD,

(30) CH₂—NH—CH₂—HetA,

(31) CH₂—N(CH₃)—CH₂—HetA,

(32) CH(CH₃)—NH—CH₂—HetA,

(33) CH(CH₃)—N(CH₃)—CH₂—HetA,

(34) HetA, or

(35) C(═O)—CH₃;

and all other variables are as originally defined or as defined in the first embodiment.

A sixth embodiment of the present invention is a compound of Formula I, or a pharmaceutically acceptable salt thereof, wherein R² is H or C₁₋₄ alkyl; and all other variables are as originally defined or as defined in any one of the preceding embodiments.

A seventh embodiment of the present invention is a compound of Formula I, or a pharmaceutically acceptable salt thereof, wherein R² is H or C₁₋₃ alkyl; and all other variables are as originally defined or as defined in any one of the preceding embodiments.

An eighth embodiment of the present invention is a compound of Formula I, or a pharmaceutically acceptable salt thereof, wherein R² is H or CH₃; and all other variables are as originally defined or as defined in any one of the preceding embodiments. In an aspect of this embodiment, R² is H.

A ninth embodiment of the present invention is a compound of Formula I, or a pharmaceutically acceptable salt thereof, wherein R³ is

(1) H,

(2) C₁₋₆ alkyl,

(3) C₁₋₆ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₆ alkylene-O—C₁₋₆ alkyl,
(e) —N(R^{a})—C(═O)—C(═O)—N(R^{a})R^{b},
(f) —HetD, or
(g) —N(R^{a})—C₁₋₆ alkylene-HetA, or

(4) C(═O)—C₁₋₆ alkyl;

and all other variables are as originally defined or as defined in any one of the preceding embodiments.

A tenth embodiment of the present invention is a compound of Formula I, or a pharmaceutically acceptable salt thereof, wherein R³ is:

(1) H,

(2) C₁₋₄ alkyl,

(3) C(═O)—C₁₋₄ alkyl,

(4) CO₂H,

(5) C(═O)—O—C₁₋₄ alkyl,

(6) C(═O)N(R^{a})R^{b}, or

(7) C(═O)—HetF;

and all other variables are as originally defined or as defined in any one of the preceding embodiments.

An eleventh embodiment of the present invention is a compound of Formula I, or a pharmaceutically acceptable salt thereof, wherein R³ is:

(1) H,

(2) C₁₋₃ alkyl,

(3) C(═O)—C₁₋₃ alkyl,

(4) CO₂H,

(5) C(═O)—O—C₁₋₃ alkyl, or

(6) C(═O)N(R^{a})R^{b};

and all other variables are as originally defined or as defined in any one of the preceding embodiments.

A twelfth embodiment of the present invention is a compound of Formula I, or a pharmaceutically acceptable salt thereof, wherein R³ is:

(1) H,

(2) CH₃,

(3) C(═O)—CH₃,

(4) CO₂H,

(5) C(═O)—O—CH₃,

(6) C(═O)N(H)CH₃, or

(7) C(═O)N(CH₃)₂;

and all other variables are as originally defined or as defined in any one of the preceding embodiments.

A thirteenth embodiment of the present invention is a compound of Formula I, or a pharmaceutically acceptable salt thereof, wherein R³ is H, C₁₋₃ alkyl, or C(═O)—C₁₋₃ alkyl; and all other variables are as originally defined or as defined in any one of the preceding embodiments.

A fourteenth embodiment of the present invention is a compound of Formula I, or a pharmaceutically acceptable salt thereof, wherein R³ is H, CH₃, or C(═O)—CH₃; and all other variables are as originally defined or as defined in any one of the preceding embodiments. In an aspect of this embodiment, R³ is H or CH₃. In another aspect, R³ is H.

A fifteenth embodiment of the present invention is a compound of Formula I, or a pharmaceutically acceptable salt thereof, wherein R⁴ is:

(1) H,

(2) C₁₋₄ alkyl, or

(3) C₁₋₄ alkyl substituted with:
(a) —N(R^{a})R^{b},
(b) —N(R^{a})—C(═O)—R^{b},
(c) —N(R^{a})—SO₂R^{b};
(d) —N(R^{a})—C₁₋₃ alkylene-O—C₁₋₄ alkyl,
(e) —N(R^{a})—C(═O)—C(═O)—N(R^{a})R^{b},
(f) —HetD, or
(g) —N(R^{a})—C₁₋₃ alkylene-HetA;
   and all other variables are as originally defined or as defined in any one of the preceding embodiments.

A sixteenth embodiment of the present invention is a compound of Formula I, or a pharmaceutically acceptable salt thereof, wherein R⁴ is:

(1) H,

(2) C₁₋₃ alkyl, or

(3) C₁₋₃ alkyl substituted with:
(a) —N(R^{a})R^{b},
(b) —N(R^{a})—C(═O)—R^{b},
(c) —N(R^{a})—SO₂R^{b},
(d) —N(R^{a})—C₁₋₃ alkylene-O—C₁₋₃ alkyl,
(e) —N(R^{a})—C(═O)—C(═O)—N(R^{a})R^{b},
(f) —HetD, or
(g) —N(R^{a})—C₁₋₃ alkylene-HetA;
   and all other variables are as originally defined or as defined in any one of the preceding embodiments.

A seventeenth embodiment of the present invention is a compound of Formula I, or a pharmaceutically acceptable salt thereof, wherein R⁴ is:

(1) H,

(2) C₁₋₃ alkyl,

(3) CH₂—N(R^{a})R^{b},

(4) CH(CH₃)—N(R^{a})R^{b},

(5) CH₂—N(R^{a})—C(═O)—R^{b},

(6) CH(CH₃)—N(R^{a})—C(═O)—R^{b},

(7) CH₂—HetD, or

(8) CH(CH₃)—HetD.

and all other variables are as originally defined or as defined in any one of the preceding embodiments.

An eighteenth embodiment of the present invention is a compound of Formula I, or a pharmaceutically acceptable salt thereof, wherein R⁴ is:

(1) H,

(2) CH₃,

(3) CH₂—NH(CH₃),

(4) CH(CH₃)—NH(CH₃),

(5) CH₂—N(CH₃)₂,

(6) CH(CH₃)—N(CH₃)₂,

(7) CH₂—N(CH₃)—C(═O)—CH₃,

(8) CH(CH₃)—N(CH₃)—C(═O)—CH₃, or

(9) CH₂—HetD;

and all other variables are as originally defined or as defined in any one of the preceding embodiments.

A nineteenth embodiment of the present invention is a compound of Formula I, or a pharmaceutically acceptable salt thereof, wherein R⁵ is:

(1) H,

(2) C₁₋₄ alkyl, or

(3) C₁₋₄ alkyl substituted with:
(a) —CO₂H,
(b) —C(═O)—O—C₁₋₄ alkyl,
(c) —N(R^{a})R^{b},
(d) —C(═O)N(R^{a})R^{b},
(e) —N(R^{a})—C(═O)—C(═O)—N(R^{a})R^{b},
(f) —HetF,
(g) —C(═O)—HetF, or
(h) —N(R^{a})—C(═O)—C(═O)—HetF;
   and all other variables are as originally defined or as defined in any one of the preceding embodiments.

A twentieth embodiment of the present invention is a compound of Formula I, or a pharmaceutically acceptable salt thereof, wherein R⁵ is:

(1) H,

(2) C₁₋₃ alkyl,

(3) CH₂CO₂H,

(4) CH₂C(═O)—O—C₁₋₄ alkyl,

(5) (CH₂)₁₋₂N(R^{a})R^{b},

(6) CH₂C(═O)N(R^{a})R^{b},

(7) (CH₂)₁₋₂N(R^{a})—C(═O)—C(═O)—N(R^{a})R^{b},

(8) (CH₂)₁₋₂—HetF,

(9) CH₂C(═O)—HetF, or

(10) (CH₂)₁₋₂N(R^{a})—C(═O)—C(═O)—HetF;

and all other variables are as originally defined or as defined in any one of the preceding embodiments.

A twenty-first embodiment of the present invention is a compound of Formula I, or a pharmaceutically acceptable salt thereof, wherein R⁵ is:

(1) H,

(2) CH₃,

(3) CH₂CO₂H,

(4) CH₂CO₂CH₃,

(5) CH₂CO₂CH₂CH₃,

(6) (CH₂)₁₋₂N(H)CH₃,

(7) (CH₂)₁₋₂N(CH₃)₂,

(8) CH₂C(═O)N(H)CH₃,

(9) CH₂C(═O)N(CH₃)₂, or

(10) (CH₂)₁₋₂—HetF;

and all other variables are as originally defined or as defined in any one of the preceding embodiments.

A twenty-second embodiment of the present invention is a compound of Formula I, or a pharmaceutically acceptable salt thereof, wherein R⁶ is H or C₁₋₄ alkyl; and all other variables are as originally defined or as defined in any one of the preceding embodiments.

A twenty-third embodiment of the present invention is a compound of Formula I, or a pharmaceutically acceptable salt thereof, wherein R⁶ is H or C₁₋₃ alkyl; and all other variables are as originally defined or as defined in any one of the preceding embodiments.

A twenty-fourth embodiment of the present invention is a compound of Formula I, or a pharmaceutically acceptable salt thereof, wherein R⁶ is H or CH₃; and all other variables are as originally defined or as defined in any one of the preceding embodiments. In an aspect of this embodiment, R⁶ is H.

A twenty-fifth embodiment of the present invention is a compound of Formula I, or a pharmaceutically acceptable salt thereof, wherein R⁵ and R⁶ are each independently H or C₁₋₄ alkyl; and all other variables are as originally defined or as defined in any one of the preceding embodiments.

A twenty-sixth embodiment of the present invention is a compound of Formula I, or a pharmaceutically acceptable salt thereof, wherein R⁵ and R⁶ are each independently H or C₁₋₃ alkyl; and all other variables are as originally defined or as defined in any one of the preceding embodiments.

A twenty-seventh embodiment of the present invention is a compound of Formula I, or a pharmaceutically acceptable salt thereof, wherein R⁵ and R⁶ are each independently H or CH₃; and all other variables are as originally defined or as defined in any one of the preceding embodiments.

A twenty-eighth embodiment of the present invention is a compound of Formula I, or a pharmaceutically acceptable salt thereof, wherein R⁵ and R⁶ are both H; and all other variables are as originally defined or as defined in any one of the preceding embodiments.

A twenty-ninth embodiment of the present invention is a compound of Formula I, or a pharmaceutically acceptable salt thereof, wherein HetA is:
(A) a 5- or 6-membered heteroaromatic ring containing a total of from 1 to 3 heteroatoms independently selected from zero to 3 N atoms, zero or 1 O atom, and zero or 1 S atom; wherein the heteroaromatic ring is attached to the rest of the compound via a carbon atom in the ring, and wherein the heteroaromatic ring is:
   (i) optionally substituted with 1 or 2 substituents each of which is independently a —C₁₋₃ alkyl; and
   (ii) optionally substituted with phenyl or —CH₂-phenyl; or
(B) a 9- or 10-membered aromatic heterobicyclic fused ring system containing a total of from 1 to 4 heteroatoms independently selected from 1 to 4 N atoms, zero or 1 O atom, and zero or 1 S atom; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
   (i) optionally substituted with 1 or 2 substituents each of which is independently a —C₁₋₃ alkyl; and
   (ii) optionally substituted with phenyl or —CH₂-phenyl; and all other variables are as originally defined or as defined in any one of the preceding embodiments.

A thirtieth embodiment of the present invention is a compound of Formula I, or a pharmaceutically acceptable salt thereof, wherein HetA is a heteroaromatic ring selected from the group consisting of oxadiazolyl, thiophenyl (alternatively referred to in the art as “thienyl”), pyrazolyl, thiazolyl, isothiazolyl, oxazolyl, isoxazolyl, imidazolyl, pyridinyl, pyrimidinyl, pyrazinyl, and pyridoimidazolyl; wherein the heteroaromatic ring is attached to the rest of the compound via a carbon atom in the ring, and wherein the heteroaromatic ring is optionally substituted with methyl or phenyl; and all other variables are as originally defined or as defined in any one of the preceding embodiments.

A thirty-first embodiment of the present invention is a compound of Formula I, or a pharmaceutically acceptable salt thereof, wherein HetD is a 5- or 6-membered saturated heterocyclic ring containing a total of from 1 to 3 heteroatoms independently selected from 1 to 3 N atoms, zero or 1 O atom, and zero or 1 S atom, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is: (i) optionally substituted with —C₁₋₃ alkyl, —(CH₂)₁₋₂—NH(—C₁₋₃ alkyl), —(CH₂)₁₋₂—N(—C₁₋₃ alkyl)₂ or —C(═O)O—C₁₋₃ alkyl; and (ii) optionally substituted with phenyl, —CH₂-phenyl, HetE, or —(CH₂)₁₋₂—HetE; wherein HetE is (i) a 5- or 6-membered heteroaromatic ring containing a total of from 1 to 3 heteroatoms independently selected from zero to 3 N atoms, zero or 1 O atom, and zero or 1 S atom or (ii) a 5- or 6-membered saturated heterocyclic ring containing a total of from 1 to 3 heteroatoms independently selected from 1 to 3 N atoms, zero or 1 O atom, and zero or 1 S atom; and all other variables are as originally defined or as defined in any one of the preceding embodiments.

A thirty-second embodiment of the present invention is a compound of Formula I, or a pharmaceutically acceptable salt thereof, wherein HetD is a heterocyclic ring selected from the group consisting of pyrrolidinyl, morpholinyl, piperidinyl, piperazinyl, 4-methylpiperazinyl, and piperidinyl fused with a benzene ring; wherein the heterocyclic ring is attached to the rest of the compound via a N atom in the ring; and all other variables are as originally defined or as defined in any one of the preceding embodiments.

A thirty-third embodiment of the present invention is a compound of Formula I, or a pharmaceutically acceptable salt thereof, wherein HetD has the same definition as in the thirty-second embodiment except that 4-methylpiperazinyl is excluded therefrom.

A thirty-fourth embodiment of the present invention is a compound of Formula I, or a pharmaceutically acceptable salt thereof, wherein each HetF is independently a 5- or 6-membered saturated heterocyclic ring containing 1 or 2 N atoms, zero or 1 O atom, and zero or 1 S atom, wherein any ring S atom is optionally oxidized to SO or SO₂, and wherein the heterocyclic ring is attached to the rest of the compound via a N atom in the ring, and wherein the heterocyclic ring is optionally substituted with 1 or 2 substituents each of which is independently a —C₁₋₄ alkyl; and all other variables are as originally defined or as defined in any one of the preceding embodiments.

A thirty-fifth embodiment of the present invention is a compound of Formula I, or a pharmaceutically acceptable salt thereof, wherein each HetF is independently a heterocyclic ring selected from the group consisting of pyrrolidinyl, morpholinyl, thiomorpholinyl, piperidinyl, piperazinyl, and 4-methylpiperazinyl, wherein the heterocyclic ring is attached to the rest of the compound via a N atom in the ring; and all other variables are as originally defined or as defined in any one of the preceding embodiments.

A thirty-sixth embodiment of the present invention is a compound of Formula I, or a pharmaceutically acceptable salt thereof, wherein R⁷ is H, C₁₋₄ alkyl, or C₁₋₄ alkyl substituted with T, wherein T is phenyl, naphthyl, quinolinyl, or isoquinolinyl, wherein the phenyl, naphthyl, quinolinyl, or isoquinolinyl is optionally substituted with from 1 to 3 substituents each of which is independently halo, —C₁₋₄ alkyl, —O—C₁₋₁₄ alkyl, —C₁₋₄ fluoroalkyl, —SO₂—C₁₋₁₄ alkyl, —C(═O)—NH(—C₁₋₄ alkyl), or —C(═O)—N(—C₁₋₄ alkyl)₂, or HetC; and all other variables are as originally defined or as defined in any one of the preceding embodiments.

A thirty-seventh embodiment of the present invention is a compound of Formula I, or a pharmaceutically acceptable salt thereof, wherein R⁷ is H, C₁₋₃ alkyl, or CH₂-T, wherein T is phenyl which is optionally substituted with from 1 to 3 substituents each of which is independently halo, —C₁₋₃ alkyl, —O—C₁₋₃ alkyl, —C₁₋₃ fluoroalkyl, —SO₂—C₁₋₃ alkyl, —C(═O)—NH(—C₁₋₃ alkyl), or —C(═O)—N(—C₁₋₃ alkyl)₂, or HetC; and all other variables are as originally defined or as defined in any one of the preceding embodiments.

A thirty-eighth embodiment of the present invention is a compound of Formula I, or a pharmaceutically acceptable salt thereof, wherein R⁷ is CH₂-T, wherein T is phenyl which is optionally substituted with from 1 to 3 substituents each of which is independently chloro, bromo, fluoro, CH₃, OCH₃, CF₃, SO₂CH₃, C(═O)NH(CH₃, C(═O)N(CH₃)₂, or oxadiazolyl; and all other variables are as originally defined or as defined in any one of the preceding embodiments.

A thirty-ninth embodiment of the present invention is a compound of Formula I, or a pharmaceutically acceptable salt thereof, wherein R⁷ is CH₂-T; and wherein T is:

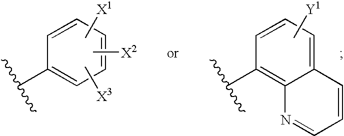

wherein X¹, X² and X³ are each independently selected from the group consisting of —H, halo, —C₁₋₄ alkyl, —O—C₁₋₄ alkyl, —C₁₋₄ fluoroalkyl, —SO₂—C₁₋₁₄ alkyl, —C(═O)—NH(—C₁₋₄ alkyl), —C(═O)—N(—C₁₋₄ alkyl)₂, and HetC; Y¹ is —H, halo, —C₁₋₄ alkyl, or —C₁₋₄ fluoroalkyl; and all other variables are as originally defined or as defined in any one of the preceding embodiments. In an aspect of this embodiment, HetC in the definition of X¹, X² and X³ is a 5- or 6-membered heteroaromatic ring containing from 1 to 4 heteroatoms independently selected from N, O and S, wherein the heteroaromatic ring is optionally substituted with 1 or 2 substituents each of which is independently a —C₁₋₃ alkyl. In another aspect of this embodiment, HetC in the definition of X¹, X² and X³ is selected from the group consisting of oxadiazolyl, thiophenyl, pyrazolyl, thiazolyl, isothiazolyl, oxazolyl, isoxazolyl, imidazolyl, pyridinyl, pyrimidinyl, pyrazinyl, and pyridoimidazolyl; wherein the heteroaromatic ring is attached to the rest of the compound via a carbon atom in the ring, and wherein the heteroaromatic ring is optionally substituted with methyl;

A fortieth embodiment of the present invention is a compound of Formula I, or a pharmaceutically acceptable salt thereof, wherein R⁷ is CH₂-T; and wherein T is:

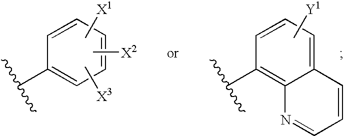

X¹ is fluoro, chloro, methyl, trifluoromethyl, methoxy, —SO₂CH₃, —C(═O)—NH(CH₃), —C(═O)—N(CH₃)₂, or oxadiazolyl; X² and X³ are each independently selected from the group consisting of —H, fluoro, chloro, methyl, trifluoromethyl, methoxy, —SO₂CH₃, —C(═O)—NH(CH₃), and —C(═O)—N(CH₃)₂; Y¹ is —H, fluoro, chloro, methyl, or trifluoromethyl; and all other variables are as originally defined or as defined in any one of the preceding embodiments.

A forty-first embodiment of the present invention is a compound of Formula I, or a pharmaceutically acceptable salt thereof, wherein R⁷ is CH₂-T; and wherein T is 4-fluorophenyl, 4-fluoro-3-methylphenyl, or 3-chloro-4-fluorophenyl; and all other variables are as originally defined or as defined in any one of the preceding embodiments.

A forty-second embodiment of the present invention is a compound of Formula I, or a pharmaceutically acceptable salt thereof, wherein R⁷ is CH₂-T; and wherein T is 4-fluorophenyl; and all other variables are as originally defined or as defined in any one of the preceding embodiments.

A forty-third embodiment of the present invention is a compound of Formula I, or a pharmaceutically acceptable salt thereof, wherein HetC is a 5- or 6-membered heteroaromatic ring containing a total of 1 to 4 heteroatoms independently selected from 1 to 4 N atoms, zero or 1 O atom, and zero or 1 S atom, wherein the heteroaromatic ring is attached to the rest of the compound via a carbon atom in the ring, and wherein the heteroaromatic ring is optionally substituted with 1 or 2 substituents each of which is independently a —C₁₋₄ alkyl; and all other variables are as originally defined or as defined in any one of the preceding embodiments.

A forty-fourth embodiment of the present invention is a compound of Formula I, or a pharmaceutically acceptable salt thereof, wherein R⁴ and R⁵ together with the carbon atoms to which each is attached and the fused ring N atom therebetween form a ring such that the compound of Formula I is a compound of Formula Ia1 or Ib1:

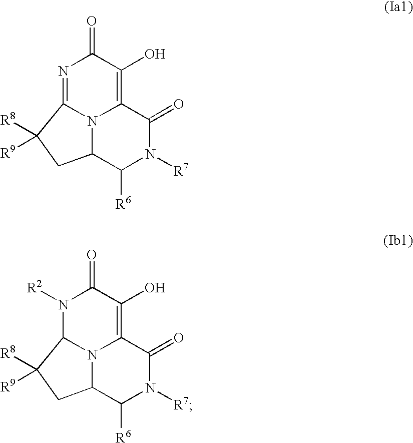

and all other variables are as originally defined or as defined in any one of the preceding embodiments.

A forty-fifth embodiment of the present invention is a compound of Formula I, or a pharmaceutically acceptable salt thereof, wherein R⁸ is: (1) H, (2) C₁₋₄ alkyl, (3) N(R^{a})R^{b}, (4) N(R^{a})—CO₂R^{b}, (5) N(R^{a})—C(═O)—C(═O)—N(R^{a})R^{b}, (6) HetF, or (7) N(R^{a})—C(═O)—C(═O)—HetF; and all other variables are as originally defined or as defined in any one of the preceding embodiments.

A forty-sixth embodiment of the present invention is a compound of Formula I, or a pharmaceutically acceptable salt thereof, wherein R⁸ is: (1) H, (2) C₁₋₃ alkyl, (3) N(R^{a})R^{b}, (4) N(R^{a})—C(═O)—O—C₁₋₄ alkyl, (5) N(R^{a})—C(═O)—C(═O)—N(R^{a})R^{b}, (6) HetF, or (7) N(R^{a})—C(═O)—C(═O)—HetF; and all other variables are as originally defined or as defined in any one of the preceding embodiments.

A forty-seventh embodiment of the present invention is a compound of Formula I, or a pharmaceutically acceptable salt thereof, wherein R⁸ is: (1) H, (2) CH₃, (3) N(H)CH₃, (4) N(CH₃)₂, (5) N(CH₃)—C(═O)—O—C₁₋₄ alkyl, (6) N(CH₃)—C(═O)—C(═O)—N(H)CH₃, (7) N(CH₃)—C(═O)—C(═O)—N(CH₃)₂, (8) HetF, or (9) N(CH₃)—C(═O)—C(═O)—HetF; and all other variables are as originally defined or as defined in any one of the preceding embodiments.

A forty-eighth embodiment of the present invention is a compound of Formula I, or a pharmaceutically acceptable salt thereof, wherein R⁹ is H, C₁₋₄ alkyl, or C₁₋₄ alkyl substituted with U, wherein U is phenyl, naphthyl, quinolinyl, or isoquinolinyl, wherein the phenyl, naphthyl, quinolinyl, or isoquinolinyl is optionally substituted with from 1 to 3 substituents each of which is independently halo, —C₁₋₄ alkyl, —O—C₁₋₄ alkyl, —C₁₋₄ fluoroalkyl, —SO₂—C₁₋₄ alkyl, —C(═O)—NH(—C₁₋₄ alkyl), —C(═O)—N(—C₁₋₄ alkyl)₂, or HetC; and all other variables are as originally defined or as defined in any one of the preceding embodiments.

A forty-ninth embodiment of the present invention is a compound of Formula I, or a pharmaceutically acceptable salt thereof, wherein R⁹ is H, C₁₋₃ alkyl, or CH₂-U, wherein U is phenyl which is optionally substituted with from 1 to 3 substituents each of which is independently halo, —C₁₋₃ alkyl, —O—C₁₋₃ alkyl, —C₁₋₃ fluoroalkyl, —SO₂—C₁₋₃ alkyl, —C(═O)—NH(—C₁₋₃ alkyl), —C(═O)—N(—C₁₋₃ alkyl)₂, or HetC; and all other variables are as originally defined or as defined in any one of the preceding embodiments.

A fiftieth embodiment of the present invention is a compound of Formula I, or a pharmaceutically acceptable salt thereof, wherein R⁹ is H, CH₃, or CH₂-U, wherein U is phenyl which is optionally substituted with from 1 to 3 substituents each of which is independently chloro, bromo, fluoro, CH₃, OCH₃, CF₃, SO₂CH₃, C(═O)NH(CH₃, C(═O)N(CH₃)₂, or oxadiazolyl; and all other variables are as originally defined or as defined in any one of the preceding embodiments.

A fifty-first embodiment of the present invention is a compound of Formula I, or a pharmaceutically acceptable salt thereof, wherein T in the definition of R⁷ and U in the definition of R⁹ are the same; and all other variables are as originally defined or as defined in any one of the preceding embodiments. In an aspect of this embodiment, R⁷ is CH₂-T and R⁹ is CH₂-U, wherein T=U, wherein T is as originally defined or as defined in a previous embodiment.

A fifty-second embodiment of the present invention is a compound of Formula I, or a pharmaceutically acceptable salt thereof, wherein R⁹ is H; and all other variables are as originally defined or as defined in any one of the preceding embodiments. In an aspect of this embodiment, R⁹ is H and R⁷ is C₁₋₆ alkyl substituted with T, or is C₁₋₄ alkyl substituted with T or is CH₂-T, wherein T is as originally defined or as defined in a previous embodiment.

A fifty-third embodiment of the present invention is a compound of Formula I, or a pharmaceutically acceptable salt thereof, wherein R¹⁰ is H or C₁₋₄ alkyl; and all other variables are as originally defined or as defined in any one of the preceding embodiments.

A fifty-fourth embodiment of the present invention is a compound of Formula I, or a pharmaceutically acceptable salt thereof, wherein R¹⁰ is H; and all other variables are as originally defined or as defined in any one of the preceding embodiments.

A fifty-fifth embodiment of the present invention is a compound of Formula I, wherein each R^{a} and R^{b} is independently H or C₁₋₄ alkyl; and all other variables are as originally defined or as defined in any one of the preceding embodiments.

A fifty-sixth embodiment of the present invention is a compound of Formula I, wherein each R^{a} and R^{b} is independently H or C₁₋₃ alkyl; and all other variables are as originally defined or as defined in any one of the preceding embodiments.

A fifty-seventh embodiment of the present invention is a compound of Formula I, wherein each R^{a} and R^{b} is independently H or methyl; and all other variables are as originally defined or as defined in any one of the preceding embodiments.

A first class of the present invention includes compounds of Formula IIa, and pharmaceutically acceptable salts thereof:

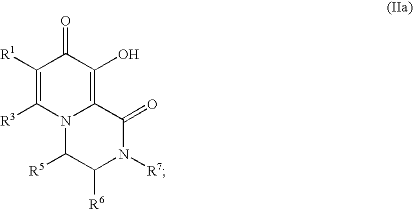

wherein R¹, R³, R⁵, R⁶ and R⁷ are each independently as originally defined above or as defined in any one of the foregoing embodiments.

A second class of the present invention includes compounds of Formula IIa, and pharmaceutically acceptable salts thereof:

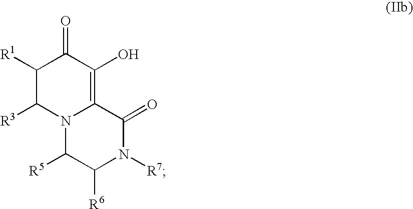

wherein R¹, R³, R⁵, R⁶ and R⁷ are each independently as originally defined above or as defined in any one of the foregoing embodiments.

A third class of the present invention includes compounds of Formula IIIa, and pharmaceutically acceptable salts thereof:

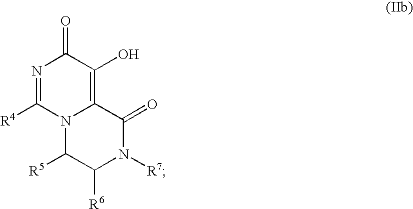

wherein R⁴, R⁵, R⁶ and R⁷ are each independently as originally defined above or as defined in any one of the foregoing embodiments.

A fourth class of the present invention includes compounds of Formula IIIb, and pharmaceutically acceptable salts thereof:

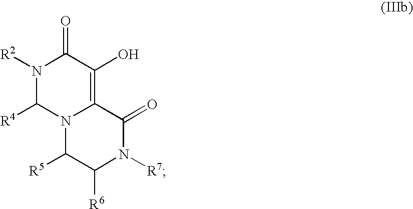

wherein R², R⁴, R⁵, R⁶ and R⁷ are each independently as originally defined above or as defined in any one of the foregoing embodiments.

A fifth class of the present invention includes compounds of Formula I, and pharmaceutically acceptable salts thereof, wherein R¹ is as defined in the third embodiment; R² is as defined in the seventh embodiment; R³ is as defined in the thirteenth embodiment; R⁴ is as defined in the sixteenth embodiment; R⁵ and R⁶ are as defined in the twenty-sixth embodiment; HetA is as defined in the twenty-ninth embodiment; HetD is as defined in the thirty-first embodiment; R^{a} and R^{b} are as defined in the fifty-sixth embodiment; and all other variables are as originally defined above or as defined in any one of the foregoing embodiments.

A sub-class of the fifth class includes compounds of Formula I, and pharmaceutically acceptable salts thereof, wherein R¹ is as defined in the fourth embodiment; R⁴ is as defined in the seventeenth embodiment; and all other variables are as defined in the fifth class.

A sixth class of the present invention includes compounds of Formula I, and pharmaceutically acceptable salts thereof, wherein R¹ is as defined in the fifth embodiment; R² is as defined in the eighth embodiment; R³ is as defined in the fourteenth embodiment; R⁴ is as defined in the seventeenth embodiment; R⁵ and R⁶ are as defined in the twenty-seventh embodiment; HetA is as defined in the thirtieth embodiment; HetD is as defined in the thirty-third embodiment; R^{a} and R^{b} are as defined in the fifty-sixth embodiment; and all other variables are as originally defined above or as defined in any one of the foregoing embodiments. In a sub-class of the sixth class, R^{a} and R^{b} are as defined in the fifty-seventh embodiment.

A seventh class of the present invention includes compounds of Formula I, and pharmaceutically acceptable salts thereof, wherein R¹ is as defined in the second embodiment; R² is as defined in the sixth embodiment; R³ is as defined in the tenth embodiment; R⁴ is as defined in the fifteenth embodiment; R⁵ is as defined in the nineteenth embodiment; or alternatively R⁴ and R⁵ together with the carbon atoms to which each is attached and the fused ring N atom therebetween form a ring such that the compound of Formula I is a compound of Formula Ia or Ib; R⁶ is as defined in the twenty-second embodiment; R⁷ is as defined in the thirty-sixth embodiment; R⁸ is as defined in the forty-fifth embodiment; R⁹ is as defined in the forty-eighth embodiment; R¹⁰ is as defined in the fifty-third embodiment; HetA is as defined in the twenty-ninth embodiment; HetC is as defined in the forty-third embodiment; HetD is as defined in the thirty-first embodiment; HetF is as defined in the thirty-fourth embodiment; R^{a} and R^{b} are as defined in the fifty-fifth embodiment; and all other variables are as originally defined above or as defined in any one of the foregoing embodiments.

An eighth class of the present invention includes compounds of Formula I, and pharmaceutically acceptable salts thereof, wherein R¹ is as defined in the fourth embodiment; R² is as defined in the seventh embodiment; R³ is as defined in the eleventh embodiment; R⁴ is as defined in the seventeenth embodiment; R⁵ is as defined in the twentieth embodiment; or alternatively R⁴ and R⁵ together with the carbon atoms to which each is attached and the fused ring N atom therebetween form a ring such that the compound of Formula I is a compound of Formula Ia1 or Ib1 as set forth in the forty-fourth embodiment; R⁶ is as defined in the twenty-third embodiment; R⁷ is as defined in the thirty-seventh embodiment; R⁸ is as defined in the forty-sixth embodiment; R⁹ is as defined in the forty-ninth embodiment; HetA is as defined in the twenty-ninth embodiment; HetC is as defined in the forty-third embodiment; HetD is as defined in the thirty-first embodiment; HetF is as defined in the thirty-fourth embodiment; R^{a} and R^{b} are as defined in the fifty-sixth embodiment; and all other variables are as originally defined above or as defined in any one of the foregoing embodiments.

A ninth class of the present invention includes compounds of Formula I, and pharmaceutically acceptable salts thereof, wherein R¹ is as defined in the fifth embodiment; R² is as defined in the eighth embodiment; R³ is as defined in the twelfth embodiment; R⁴ is as defined in the eighteenth embodiment; R⁵ is as defined in the twenty-first embodiment; or alternatively R⁴ and R⁵ together with the carbon atoms to which each is attached and the fused ring N atom therebetween form a ring such that the compound of Formula I is a compound of Formula Ia1 or Ib1 as set forth in the forty-fourth embodiment; R⁶ is as defined in the twenty-fourth embodiment; R⁷ is as defined in the thirty-eighth embodiment; R⁸ is as defined in the forty-seventh embodiment; R⁹ is as defined in the fiftieth embodiment; HetA is as defined in the thirtieth embodiment; HetD is as defined in the thirty-second embodiment; HetF is as defined in the thirty-fifth embodiment; and all other variables are as originally defined above or as defined in any one of the foregoing embodiments.

A tenth class of the present invention includes compounds of Formula IV, and pharmaceutically acceptable salts thereof:

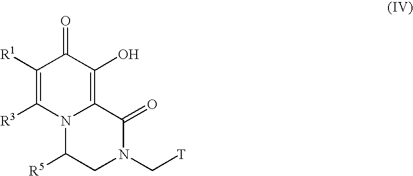

wherein R¹ is:
(1) H,
(2) C₁₋₃ alkyl,
(3) chloro,
(4) bromo,
(5) CH₂—N(R^{a})R^{b},
(6) CH(CH₃)—N(R^{a})R^{b},
(7) CH₂—N(R^{a})—C(═O)—R^{b},
(8) CH(CH₃)—N(R^{a})—C(═O)—R^{b},
(9) CH₂—N(R^{a})—SO₂R^{b},
(10) CH(CH₃)—N(R^{a})—SO₂R^{b},
(11) CH₂—N(R^{a})—C₁₋₃ alkylene-O—C₁₋₃ alkyl (e.g., CH₂—N(R^{a})—C₂₋₃ alkylene-O—C₁₋₃ alkyl),
(12) CH(CH₃)—N(R^{a})—C₁₋₃ alkylene-O—C₁₋₃ alkyl (e.g., CH(CH₃)—N(R^{a})—C₂₋₃ alkylene-O—C₁₋₃ alkyl),
(13) CH₂—N(R^{a})—C(═O)—C(═O)—N(R^{a})R^{b},
(14) CH(CH₃)—N(R^{a})—C(═O)—C(═O)—N(R^{a})R^{b},
(15) —CH₂—OH,
(16) CH(CH₃)—OH,
(17) CH₂—HetD,
(18) CH(CH₃)—HetD,
(19) CH₂—N(R^{a})—CH₂—HetA,
(20) CH(CH₃)—N(R^{a})—CH₂—HetA,
(21) HetA, or
(22) C(═O)—R^{a}; and

### R³ is:

(1) H,

(2) C₁₋₃ alkyl,

(3) C(═O)—C₁₋₃ alkyl,

(4) CO₂H,

(5) C(═O)—O—C₁₋₃ alkyl, or

(6) C(═O)N(R^{a})R^{b};

### R⁵ is:

(1) H,

(2) C₁₋₃ alkyl,

(3) CH₂CO₂H,

(4) CH₂C(═O)—O—C₁₋₄ alkyl,

(5) (CH₂)₁₋₂N(R^{a})R^{b},

(6) CH₂C(═O)N(R^{a})R^{b},

(7) (CH₂)₁₋₂N(R^{a})—C(═O)—C(═O)—N(R^{a})R^{b},

(8) (CH₂)₁₋₂—HetF,

(9) CH₂C(═O)—HetF, or

(10) (CH₂)₁₋₂N(R^{a})—C(═O)—C(═O)—HetF;

### T is

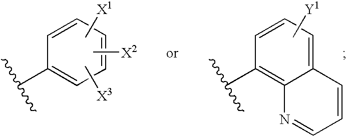

wherein X¹, X² and X³ are each independently selected from the group consisting of —H, halo, —C₁₋₄ alkyl, —O—C₁₋₄ alkyl, —C₁₋₄ fluoroalkyl, —SO₂—C₁₋₄ alkyl, —C(═O)—NH(—C₁₋₄ alkyl), —C(═O)—N(—C₁₋₄ alkyl)₂, and HetC;
Y¹ is —H, halo, —C₁₋₄ alkyl, or —C₁₋₄ fluoroalkyl;
HetA is a 5- or 6-membered heteroaromatic ring containing a total of from 1 to 3 heteroatoms independently selected from zero to 3 N atoms, zero or 1 O atom, and zero or 1 S atom; wherein the heteroaromatic ring is attached to the rest of the compound via a carbon atom in the ring, and wherein the heteroaromatic ring is (i) optionally substituted with 1 or 2 substituents each of which is independently a —C₁₋₃ alkyl and (ii) optionally substituted with phenyl or —CH₂-phenyl;
each HetC is independently a 5- or 6-membered heteroaromatic ring containing from 1 to 4 heteroatoms independently selected from N, O and S, wherein the heteroaromatic ring is optionally substituted with 1 or 2 substituents each of which is independently a —C₁₋₃ alkyl;
HetD is a 5- or 6-membered saturated heterocyclic ring containing a total of from 1 to 3 heteroatoms independently selected from 1 to 3 N atoms, zero or 1 O atom, and zero or 1 S atom, wherein any ring S atom is optionally oxidized to SO or SO₂, and wherein the heterocyclic ring is attached to the rest of the compound via a N atom in the ring, and wherein the heterocyclic ring is optionally substituted with —C₁₋₃ alkyl;
HetF is a 5- or 6-membered saturated heterocyclic ring containing 1 or 2 N atoms, zero or 1 O atom, and zero or 1 S atom, wherein any ring S atom is optionally oxidized to SO or SO₂, and wherein the heterocyclic ring is attached to the rest of the compound via a N atom in the ring, and wherein the heterocyclic ring is optionally substituted with 1 or 2 substituents each of which is independently a —C₁₋₄ alkyl;
each R^{a} is independently H or C₁₋₃ alkyl; and
each R^{b} is independently H or C₁₋₃ alkyl.

A first sub-class of the tenth class includes compounds of Formula IV, and pharmaceutically acceptable salts thereof, wherein R¹ is:
(1) H,
(2) CH₃,
(3) bromo,
(4) CH(CH₃)—N(R^{a})R^{b},
(5) CH(CH₃)—N(R^{a})—C(═O)—R^{b},
(6) CH(CH₃)—N(R^{a})—SO₂R^{b},
(7) CH(CH₃)—N(R^{a})—C₁₋₃ alkylene-O—C₁₋₁₃ alkyl (e.g., CH(CH₃)—N(R^{a})—C₂₋₃ alkylene-O—C₁₋₃ alkyl),
(8) CH(CH₃)—N(R^{a})—C(═O)—C(═O)—N(R^{a})R^{b},
(9) CH(CH₃)—OH,
(10) CH(CH₃)—HetD,
(11) CH(CH₃)—N(R^{a})—CH₂—HetA,
(12) HetA, or
(13) C(═O)CH₃;

### R³ is:

(1) H,

(2) CH₃,

(3) C(═O)—CH₃,

(4) CO₂H, or

(5) C(═O)N(CH₃)₂;

### R⁵ is:

(1) H,

(2) CH₃,

(3) CH₂CO₂H,

(4) CH₂CO₂CH₃,

(5) CH₂CO₂CH₂CH₃,

(6) (CH₂)₁₋₂N(H)CH₃,

(7) (CH₂)₁₋₂N(CH₃)₂,

(8) CH₂C(═O)N(H)CH₃,

(9) CH₂C(═O)N(CH₃)₂, or

(10) (CH₂)₁₋₂—HetF;

with the proviso that at least one of R³ and R⁵ is H;
T is 4-fluorophenyl, 4-fluoro-3-methylphenyl, or 3-chloro-4-fluorophenyl;
HetA is pyrrolyl, imidazolyl, pyridinyl, pyrimidinyl, or pyrazinyl;

### HetD is

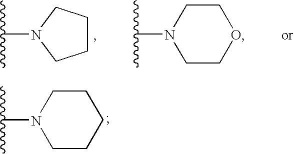

### HetF is

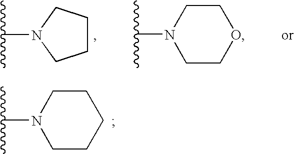

### R^{a} is H or CH₃; and

### R^{b} is CH₃ or CH(CH₃)₂.

A second subclass of the tenth class is identical to the first subclass, except that T is 4-fluorophenyl.

A third subclass of the tenth class includes compounds of Formula IVa, and pharmaceutically acceptable salts thereof:

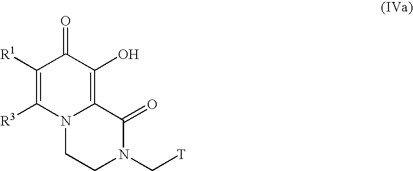

R³ is H, C₁₋₃ alkyl, or C(═O)—C₁₋₃ alkyl; and R¹, T and all variables included in the definitions of R¹ and T are as originally defined in the tenth class.

A fourth sub-class of the seventh class includes compounds of Formula IVa, and pharmaceutically acceptable salts thereof, wherein R¹ is as defined in the first sub-class of the tenth class; R³ is H, CH₃, or C(═O)—CH₃; T is 4-fluorophenyl; and R¹, HetA, HetD, R^{a}, and R^{b} are each as defined in the first sub-class of the tenth class.

An eleventh class of the present invention includes compounds of Formula V, and pharmaceutically acceptable salts thereof:

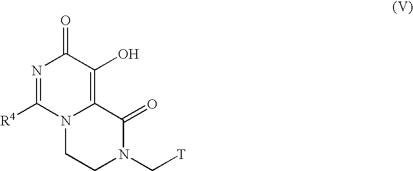

wherein R⁴ is:

(1) H,

(2) C₁₋₃ alkyl,

(3) CH₂—N(R^{a})R^{b},

(4) CH(CH₃)—N(R^{a})R^{b},

(5) CH₂—N(R^{a})—C(═O)—R^{b},

(6) CH(CH₃)—N(R^{a})—C(═O)—R^{b},

(7) CH₂—HetD, or

(8) CH(CH₃)—HetD;

and T, X¹, X², X³, Y¹, HetC, HetD, R^{a} and R^{b} are each as defined in the tenth class.

A first sub-class of the eleventh class includes compounds of Formula V, and pharmaceutically acceptable salts thereof, wherein R⁴ is:

(1) H,

(2) C₁₋₃ alkyl,

(3) CH₂—N(R^{a})R^{b},

(4) CH(CH₃)—N(R^{a})R^{b},

(5) CH₂—N(R^{a})—C(═O)—R^{b},

(6) CH(CH₃)—N(R^{a})—C(═O)—R^{b},

(7) CH₂—HetD, or

(8) CH(CH₃)—HetD;

T is 4-fluorophenyl, 4-fluoro-3-methylphenyl, or 3-chloro-4-fluorophenyl;

### HetD is

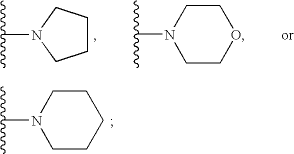

### R^{a} is H or CH₃; and

### R^{b} is CH₃.

A second sub-class of the eleventh class is identical to the first sub-class except that T is 4-fluorophenyl.

A twelfth class of the present invention includes compounds of Formula VI, and pharmaceutically acceptable salts thereof:

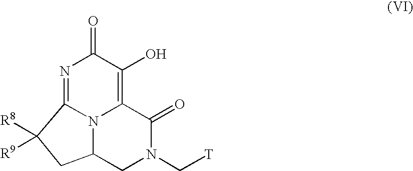

wherein

### R⁸ is:

(1) H,

(2) C₁₋₃ alkyl,

(3) N(R^{a})R^{b},

(4) N(R^{a})—C(═O)—O—C₁₋₄ alkyl,

(5) N(R^{a})—C(═O)—C(═O)—N(R^{a})R^{b},

(6) HetF, or

(7) N(R^{a})—C(═O)—C(═O)—HetF;

### R⁹ is H or CH₂-T;

and T, X¹, X², X³, Y¹, HetC, HetF, R^{a} and R^{b} are each as defined in the tenth class.

A first sub-class of the twelfth class includes compounds of Formula VI, and pharmaceutically acceptable salts thereof, wherein:

### R⁸ is:

(1) N(H)CH₃,

(2) N(CH₃)₂,

(3) N(CH₃)—C(═O)—O—C₁₋₄ alkyl,

(4) N(CH₃)—C(═O)—C(═O)—N(H)CH₃, or

(5) N(CH₃)—C(═O)—C(═O)—N(CH₃)₂,

(6) HetF, or

(7) N(CH₃)—C(═O)—C(═O)—HetF;

### R⁹ is H or CH₂-T;

T is 4-fluorophenyl, 4-fluoro-3-methylphenyl, or 3-chloro-4-fluorophenyl; and

### HetF is

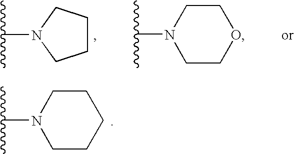

A second sub-class of the twelfth class is identical to the first sub-class, except that R⁹ is H.

A third sub-class of the twelfth class is identical to the first sub-class, except that T is 4-fluorophenyl

A fourth sub-class of the twelfth class is identical to the first sub-class, except that R⁹ is H; and T is 4-fluorophenyl

Another embodiment of the present invention is a compound, or a pharmaceutically acceptable salt thereof, selected from the group consisting of the compounds set forth in Table 1 below.

Other embodiments of the present invention include the following:

(a) A pharmaceutical composition comprising an effective amount of a compound of Formula (I) and a pharmaceutically acceptable carrier.

(b) A pharmaceutical composition which comprises the product prepared by combining (e.g., mixing) an effective amount of a compound of Formula (I) and a pharmaceutically acceptable carrier.

(c) The pharmaceutical composition of (a) or (b), further comprising an effective amount of an HIV infection/AIDS treatment agent selected from the group consisting of HIV/AIDS antiviral agents, immunomodulators, and anti-infective agents.

(d) The pharmaceutical composition of (c), wherein the HIV infection/AIDS treatment agent is an antiviral selected from the group consisting of HIV protease inhibitors, non-nucleoside HIV reverse transcriptase inhibitors, and nucleoside HIV reverse transcriptase inhibitors.

(e) A pharmaceutical combination which is (i) a compound of Formula I and (ii) an HIV infection/AIDS treatment agent selected from the group consisting of HIV/AIDS antiviral agents, immunomodulators, and anti-infective agents; wherein the compound of Formula I and the HIV infection/AIDS treatment agent are each employed in an amount that renders the combination effective for inhibiting HIV integrase, for treating or preventing infection by HIV, or for preventing, treating or delaying the onset of AIDS.

(f) The combination of (e), wherein the HIV infection/AIDS treatment agent is an antiviral selected from the group consisting of HIV protease inhibitors, non-nucleoside HIV reverse transcriptase inhibitors and nucleoside HIV reverse transcriptase inhibitors.

(g) A method of inhibiting HIV integrase in a subject in need thereof which comprises administering to the subject an effective amount of a compound of Formula I.

(h) A method of preventing or treating infection by HIV in a subject in need thereof which comprises administering to the subject an effective amount of a compound of Formula I.

(i) The method of (h), wherein the compound of Formula (I) is administered in combination with an effective amount of at least one antiviral selected from the group consisting of HIV protease inhibitors, non-nucleoside HIV reverse transcriptase inhibitors, and nucleoside HIV reverse transcriptase inhibitors.

(j) A method of preventing, treating or delaying the onset of AIDS in a subject in need thereof which comprises administering to the subject an effective amount of a compound of Formula I.

(k) The method of (j), wherein the compound is administered in combination with an effective amount of at least one antiviral selected from the group consisting of HIV protease inhibitors, non-nucleoside HIV reverse transcriptase inhibitors, and nucleoside HIV reverse transcriptase inhibitors

(l) A method of inhibiting HIV integrase in a subject in need thereof which comprises administering to the subject the pharmaceutical composition of (a), (b), (c) or (d) or the combination of (e) or (f).

(m) A method of preventing or treating infection by HIV in a subject in need thereof which comprises administering to the subject the pharmaceutical composition of (a), (b), (c) or (d) or the combination of (e) or (f).

(n) A method of preventing, treating or delaying the onset of AIDS in a subject in need thereof which comprises administering to the subject the pharmaceutical composition of (a), (b), (c) or (d) or the combination of (e) or (f).

The present invention also includes a compound of the present invention (i) for use in, (ii) for use as a medicament for, or (iii) for use in the preparation of a medicament for: (a) inhibiting HIV integrase, (b) preventing or treating infection by HIV, or (c) preventing, treating or delaying the onset of AIDS. In these uses, the compounds of the present invention can optionally be employed in combination with one or more HIV/AIDS treatment agents selected from HIV/AIDS antiviral agents, anti-infective agents, and immunomodulators.

Additional embodiments of the invention include the pharmaceutical compositions, combinations and methods set forth in (a)-(n) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkylene” refers to any linear or branched chain alkylene group (or alternatively “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Also of interest is the alkylene —CH(CH₃)—.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “C₄₋₇ azacycloalkyl” (or “C₄-C₇ azacycloalkyl”) means a saturated cyclic ring consisting of one nitrogen and from four to seven carbon atoms (i.e., pyrrolidinyl, piperidinyl, azepanyl, or octahydroazocinyl).

The term “C₃₋₆ diazacycloalkyl” (or “C₃-C₆ diazacycloalkyl”) means a saturated cyclic ring consisting of two nitrogens and from three to six carbon atoms (e.g., imidazolidinyl, pyrazolidinyl, or piperazinyl).

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth.

When any variable (e.g., R^{a} or HetC) occurs more than one time in any constituent or in Formula I or in any other formula depicting and describing compounds of the invention, its definition on each occurrence is independent of its definition at every other occurrence. Also, combinations of substituents and/or variables are permissible provided that combinations result in stable compounds.

The term “substituted” (e.g., as in “is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., aryl, a heteroaromatic ring, or a saturated heterocyclic ring) provided such ring substitution is chemically allowed and results in a stable compound.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The symbol “ [symbol] ” in front of an open bond in the structural formula of a group marks the point of attachment of the group to the rest of the molecule.

When a compound of the present invention has one or more asymmetric centers and thus can occur as an optical isomer (e.g., an enantiomer or a diastereomer), it is understood that the present invention includes all isomeric forms of the compound, singly and in mixtures.

As would be recognized by one of ordinary skill in the art, certain of the compounds of the present invention can exist as tautomers, such as the following:

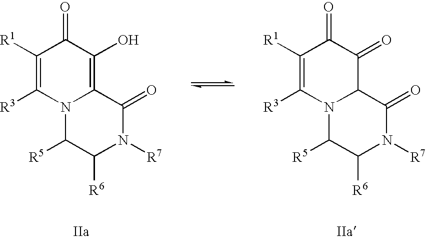

For the purposes of the present invention, a reference herein to a compound of Formula I (or Ia, Ia1, Ib, Ib1, IIa, IIb, IIIa, IIIb, IV, IVa, V, or VI) is a reference to compound I per se (or Ia, Ia1, Ib, Ib1, IIa, IIb, IIIa, IIIb, IV, IVa, V, or VI), to any one of its tautomers per se, or to mixtures thereof.

The compounds of the present inventions are useful in the inhibition of HIV integrase, the prevention or treatment of infection by human immunodeficiency virus (HIV) and the prevention, treatment or the delay in the onset of consequent pathological conditions such as AIDS. Preventing AIDS, treating AIDS, delaying the onset of AIDS, or preventing or treating infection by HIV is defined as including, but not limited to, treatment of a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by such means as blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

Compounds representative of the present invention have been tested for inhibition in an assay for the strand transfer activity of integrase. The assay is conducted in the manner described in WO 02/30930. Representative compounds of the present invention exhibit inhibition of strand transfer activity in this assay. For example, the compounds set forth in Table 1 below were tested in the integrase assay and demonstrated IC₅₀'s of about 1 micromolar or less. Further description on conducting the assay using preassembled complexes is found in Hazuda et al., J. Virol. 1997, 71: 7005-7011; Hazuda et al., Drug Design and Discovery 1997, 15: 17-24 and Hazuda et al., Science 2000, 287: 646-650.

Compounds representative of the present invention have also been tested in an assay for inhibition of acute HIV infection of T-lymphoid cells, conducted in accordance with Vacca, J. P. et al., Proc. Natl. Acad. Sci. USA 1994, 91: 4096. For example, the first thirty-two compounds set forth below in Table 1 demonstrated IC₉₅'s of less than about 20 micromolar.

The compounds of the present invention may be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of the invention mean providing the compound or a prodrug of the compound to the individual in need of treatment. When a compound of the invention or a prodrug thereof is provided in combination with one or more other active agents (e.g., antiviral agents useful for treating HIV infection or AIDS), “administration” and its variants are each understood to include concurrent and sequential provision of the compound or prodrug and other agents.

As used herein, the term “composition” is intended to encompass a product comprising the specified ingredients in the specified amounts, as well as any product which results, directly or indirectly, from combining the specified ingredients in the specified amounts.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV integrase and thereby elicit the response being sought (i.e., an “inhibition effective amount”). When the active compound (I.e., active ingredient) is administered as the salt, references to the amount of active ingredient are to the free acid or free base form of the compound.

For the purpose of inhibiting HIV integrase, preventing or treating HIV infection or preventing, treating or delaying the onset of AIDS, the compounds of the present invention, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 18^{th} edition, edited by A. R. Gennaro, Mack Publishing Co., 1990.

The compounds of this invention can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of the HIV integrase inhibitor compounds of the present invention with one or more agents useful in the treatment of HIV infection or AIDS. For example, the compounds of this invention may be effectively administered, whether at periods of pre-exposure and/or post-exposure, in combination with effective amounts of one or more HIV/AIDS antivirals, immunomodulators, antiinfectives, or vaccines useful for treating HIV infection or AIDS, such as those disclosed in Table 1 of WO 01/38332 or in the Table in WO 02/30930. Suitable HIV/AIDS antivirals for use in combination with the compounds of the present invention include, for example, HIV protease inhibitors (e.g., indinavir, atazanavir, lopinavir optionally with ritonavir, saquinavir, or nelfinavir), nucleoside HIV reverse transcriptase inhibitors (e.g., abacavir, lamivudine (3TC), zidovudine (AZT), or tenofovir), and non-nucleoside HIV reverse transcriptase inhibitors (e.g., efavirenz or nevirapine). It will be understood that the scope of combinations of the compounds of this invention with HIV/AIDS antivirals, immunomodulators, anti-infectives or vaccines is not limited to the foregoing substances or to the list in the above-referenced Tables in WO 01/38332 and WO 02/30930, but includes in principle any combination with any pharmaceutical composition useful for the treatment of AIDS. The HIV/ADS antivirals and other agents will typically be employed in these combinations in their conventional dosage ranges and regimens as reported in the art, including, for example, the dosages described in the Physicians' Desk Reference, 57^{th} edition, Thomson PDR, 2003. The dosage ranges for a compound of the invention in these combinations are the same as those set forth above.

Abbreviations used in the instant specification, particularly the Schemes and Examples, include the following:
AIDS=acquired immunodeficiency syndrome
ARC=AIDS related complex
Bn=benzyl
(BOC)₂O (or BOC₂O)=di-t-butyl carbonate
Bz=benzoate
DCM=dichloromethane
DEAD=diethylazodicarboxylate
DMAP=4-dimethylaminopyridine
DMF=N,N-dimethylformamide
DMSO=dimethylsulfoxide
EDC=1-ethyl-3-(3-dimethylaminopropyl)carbodiimide
ES=electrospray
Et=ethyl
EtOH=ethanol
EtOAc=ethyl acetate
HIV=human immunodeficiency virus
HOBT or HOBt=1-hydroxy benzotriazole hydrate
HPLC=high performance liquid chromatography
i-Pr=isopropyl
m-CPBA=meta-chloroperbenzoic acid
Me=methyl
MeOH=methanol
NBS=N-bromosuccinimide
NIS=N-iodosuccinimide
NMR=nuclear magnetic resonance
Ph=phenyl
PMB=para-methoxybenzyl
PyBOP=benzotriazol-1-yl-oxytripyrrolidinophosphonium hexafluorophosphate
RP-HPLC=reverse phase HPLC
TBS=t-butyl-dimethylsilyl
Tf₂O=triflic anhydride
TFA=trifluoroacetic acid
THf=tetrahydrofuran
TMSCN=trimethylsilyl cyanide

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

The general synthetic strategy for these compounds is outlined in Scheme 1. Essentially a functionalized heterocyclic carboxylic acid 1-1 (either a pyridine or a pyrimidine) is coupled with a secondary amine 1-0 bearing both a substituted benzyl group and a substituted protected 2-hydroxyethyl group. Once coupled, the protecting group(s) is/are removed to reveal a cyclization precursor 1-2. This key intermediate can be cyclized in a variety of conditions, such as by conversion of the hydroxyl to a suitable leaving group, (e.g. chloride) and then base mediated cyclization, or via a Mitsunobu process. These cyclizations give the crucial bicycle 1-3 which can be further synthetically elaborated to an analogue 1-4. Final deprotection yields the desired inhibitors 1-4 or 1-5.

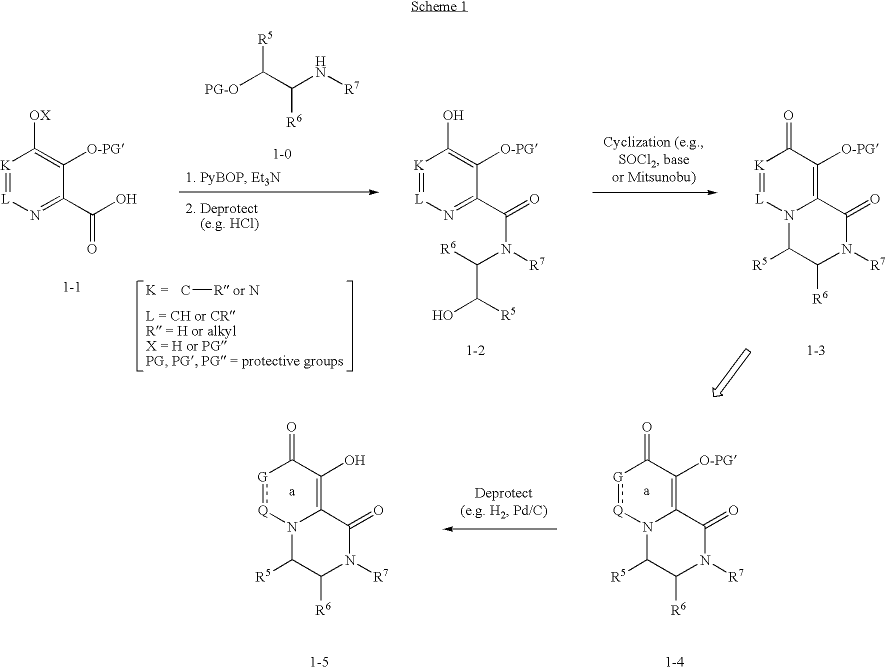

The key carboxylic acids or derivatives thereof employed as 1-1 in Scheme 1 can be readily obtained via established chemical processes (see, e.g., WO 02/06246; Sunderland et al. Inorganic Chem. 2001, 40: 6746; Piyamongkol et al., Tetrahedron 2001, 57: 3479; Boger, J. Am. Chem. Soc. 1999, 121: 2471; and Shimano, Tetrahedron Lett. 1998, 39: 4363). The secondary amines 1-0 can be readily prepared through alkylation processes (see, e.g., Michael B. Smith and Jerry March, Advanced Organic Chemistry, 5th edition, John Wiley & Sons, 2001, p. 499 and Richard Larock, Comprehensive Organic Transformations, VCH Publishers Inc., 1989 p. 397) or reductive aminations (see, e.g., R. O. Hutchins in Comprehensive Organic Synthesis, edited by B. M. Trost, Vol. 8, Pergamon Press, 1993, p. 25 and E. W. Baxter and A. B. Reitz, Organic Reactions, edited by L. E. Overman, Vol. 59, John Wiley, 2002, p. 1). Representative cyclization methods are described Seibel, Bioorg. Med. Chem. Lett. 2003, 13: 387; Mickelson et al., J. Org. Chem. 1995, 60: 4177; and Machon et al., Farmaco Ed. Sci. 1985, 40: 695-700. Suitable protecting groups and methods for removing them are described, for example, in T. W. Greene and P. G. M. Wuts, Protective Groups in Organic Synthesis, 3^{rd} Edition, Wiley-Interscience, 1999; and P. J. Kocienski, Protecting Groups, Thieme, 1994.

Schemes 2 to 15 below illustrate and expand upon the chemistry portrayed in Scheme 1. These schemes illustrate the chemistry In Scheme 2, a 3,4-dihydroxypyridine is differentially protected to give 2-1 (e.g., with benzyl and p-methoxybenzyl, although other protecting groups can be employed) and then N-oxidized and rearranged in a similar manner as described in Tetrahedron 2001, 57: 3479. After basic hydrolysis to the 2-hydroxymethyl compound 2-2, sequential oxidation (e.g., by Swern oxidation and then using sodium chlorite in the presence of sulfamic acid) will afford the aldehyde 2-3 and then the acid 2-4 (other suitable methods are described in M. Hudlicky, Oxidations in Organic Chemistry, Am. Chem. Soc., Washington, 1990). Amide coupling (e.g. with PyBOP) will give the amide 2-5 and the tert-butyldimethylsilyl and para-methoxybenzyl groups can then be removed from the alcohol (e.g., using acid although other deprotection methods are available) to give 2-6. Compound 2-6 can be cyclized by treatment with thionyl chloride in the presence of pyridine to afford the bicycle 2-7 (as described by Machon, Z. et al. Farmaco Ed. Sci., 1985, 40(9), 695-700). The benzyl group can then be removed (e.g., by hydrogenolysis) to give 2-8.

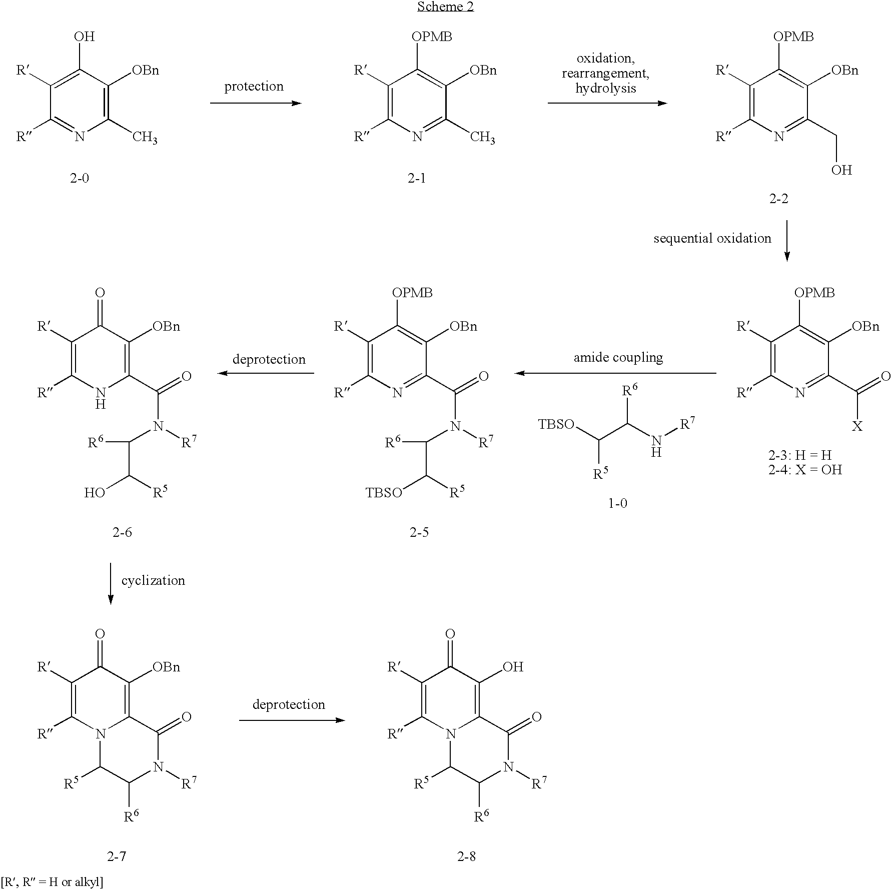

An alternative process for the preparation of these compounds is described in Scheme 3, where a suitable heterocyclic carboxylic ester 3-0 (synthesized as described, e.g., in Sunderland et al. Inorganic Chem. 2001, 40: 6746) can be hydrolyzed (e.g., KOH in EtOH with heating) to the acid 3-1, which can then be amide coupled (e.g., using PyBOP and Et₃N) and deprotected (e.g., HCl in THF) to afford 3-2. This material can be cyclized under Mitsunobu conditions (e.g., as described by Seibel, Bioorg. Med. Chem. Lett. 2003, 13: 387; and Mickelson et al., J. Org. Chem. 1995, 60: 4177) to give the desired bicycle 3-3. Hydrogenation (e.g., H₂, Pd/C, MeOH) then gives structures of the type either 2-(benzyl)-9-hydroxy-6-alkyl-3,4-dihydro-2H-pyrazino[1,2-c]pyrimidine-1,8-dione 3-5 and/or 2-(benzyl)-9-hydroxy-6-alkyl-3,4,6,7-tetrahydro-2H-pyrazino[1,2-c]pyrimidine-1,8-dione 3-4 depending on conditions used.

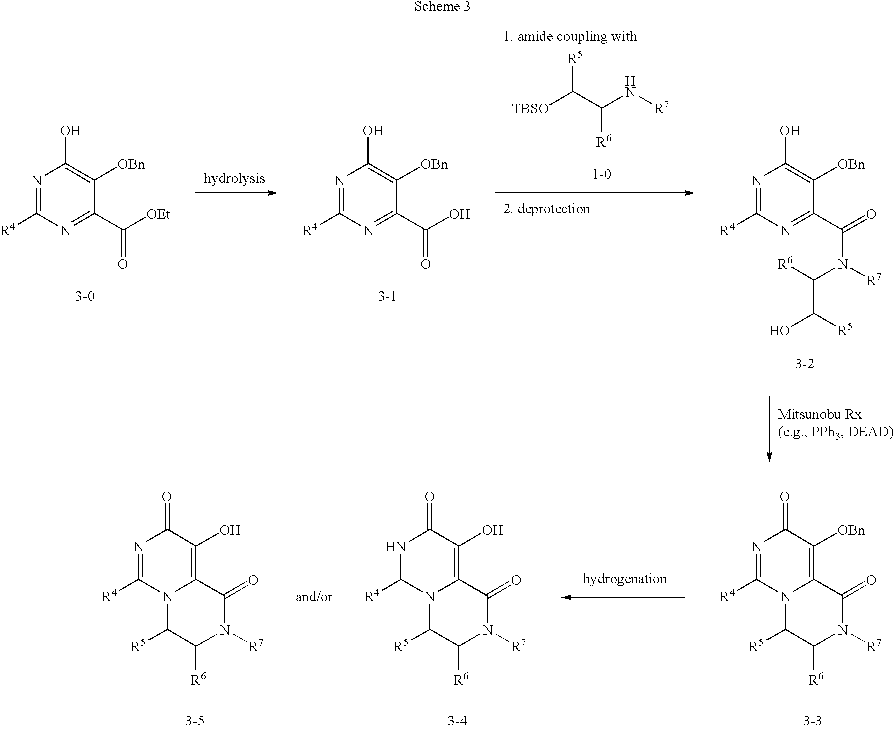

The core scaffold can be further manipulated as shown in Scheme 4. Halogenation (e.g., using NIS with mCPBA or using Br₂) can be performed to give the intermediate 4-1. This intermediate can then be cross-coupled (e.g., using a Stille reaction with an appropriate organostannane under Pd(0) catalysis as described in J. Tsuji, Palladium Reagents and Catalysts, Wiley, 1997, p. 228) to introduce a substituent at the C-7 position. Subsequent deprotection to remove benzyl will afford 4-2.

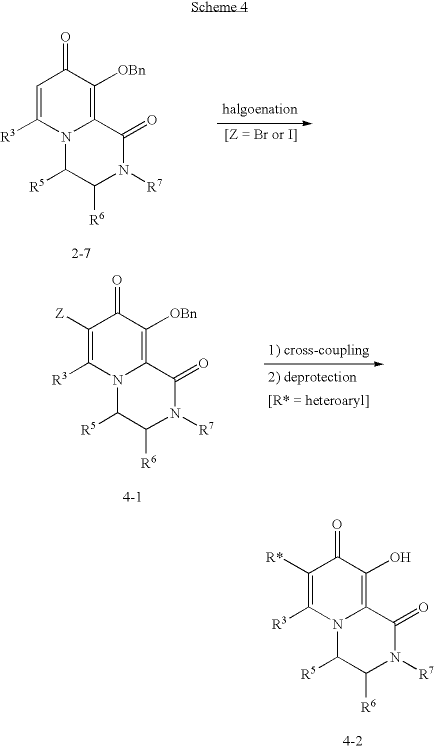

A modification of this procedure is depicted in Scheme 5 whereby the halogen intermediate 4-1 can be cross-coupled with a stannylated alkyl enol ether (see Chemistry Lett. 1989, 1959-62). The resulting intermediate enol ether can then be hydrolyzed with acid to give the corresponding ketone 5-1, which can then be deprotected (e.g., HCl in THF with heating) to afford the compound 5-2.

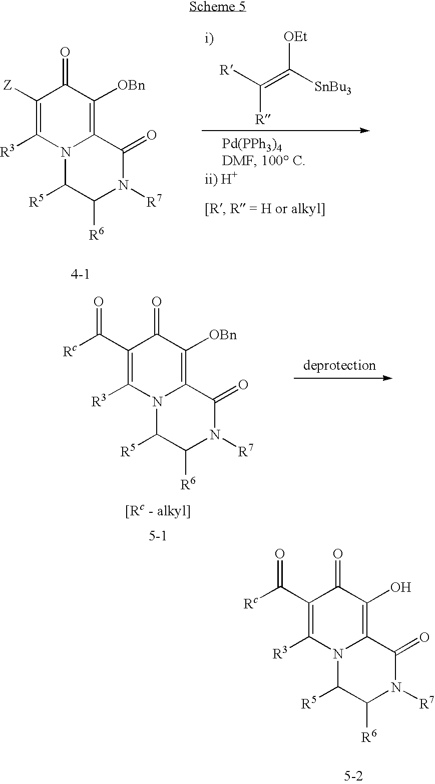

The ketone 5-1 can be readily transformed into the corresponding alcohol 6-1 as depicted in Scheme 6 using a suitable reducing agent (e.g., sodium borohydride or agents described in M. Hudlicky, Reductions in Organic Chemistry, A.C.S., Washington, 1996). Subsequent deprotection of 6-1 (e.g., H₂ with Pd/C) will then afford 6-2.

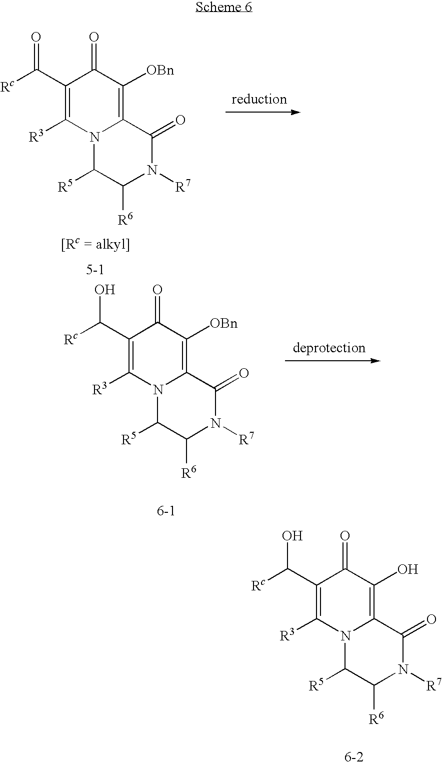

The ketone 5-1 can also be transformed into an amine 7-1 as described in Scheme 7 utilizing a reductive amination (e.g., treating 7-1 with excess amine in MeOH in the presence of sodium cyanoborohydride). Suitable reductive amination methods are described in, e.g., R. O. Hutchins in Comprehensive Organic Synthesis, edited by B. M. Trost, Pergamon Press, Vol. 8, 1993, p. 25 and E. W. Baxter and A. B. Reitz, Organic Reactions, edited by L. E. Overman, Vol. 59, John Wiley, 2002, p. 1. The amine 7-1 can then either be deprotected (e.g., by hydrogenation) to provide compound 7-2. The amine can also be further reacted with a capping group (Cap-Cl). Suitable capping groups include acyl chlorides, sulfonyl chlorides, and carbamyl chlorides and the like. Other acid derivatives in combination with an appropriate activating reagent (e.g., a carboxylic and coupling reagent such as EDC/HOBt or PyBOP) are suitable for use in this reaction. These reactions are conducted in the presence of a base (e.g., triethylamine) to scavenge the HCl by-product. Subsequent deprotection (e.g., H₂ with Pd/C) will then afford compound 7-3.

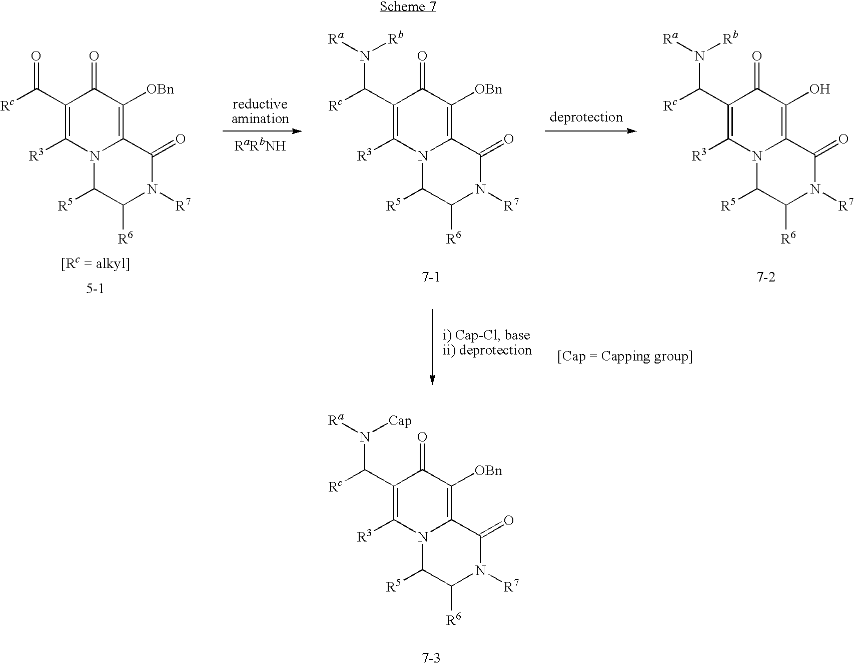

An alternative method for functionalizing the core scaffold is depicted in Scheme 8, using the propensity of the carbon atom at the C-6 position of the bicycle to undergo radical bromination. Treatment of 8-1 with a brominating agent (e.g., N-bromosuccinimide in the presence of catalytic benzoyl peroxide) will afford the bromine derivative 8-2. The bromine can then be displaced by an amine (e.g., using chemistry described in, for example, Michael B. Smith and Jerry March, Advanced Organic Chemistry, 5th edition, John Wiley & Sons, 2001, p. 499 and in Richard Larock, Comprehensive Organic Transformations, VCH Publishers Inc, 1989, p. 397) to afford 8-3. The amine can then either be deprotected (e.g., by hydrogenation) to give 8-4, or the amine can be further reacted with a capping group (Cap-Cl) in the manner described above in Scheme 7. Subsequent deprotection (e.g., H₂ with Pd/C) will then afford compound 8-5.

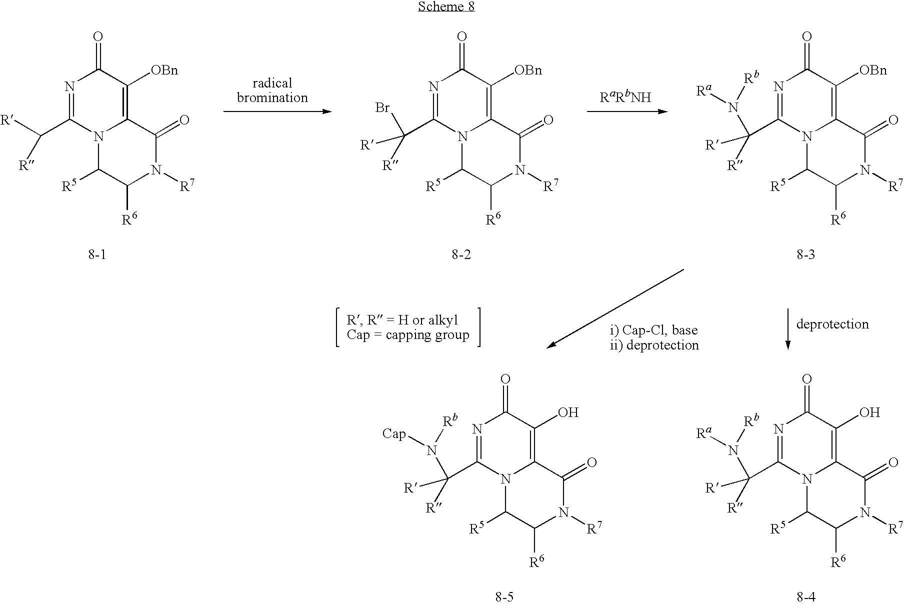

Scheme 9 below illustrates and expands upon the chemistry portrayed in Scheme 2. Here the substituted pyridine 9-1 can be N-oxidized and rearranged in a manner similar to that described in Tetrahedron 2001, 57: 3479 to yield the 2-acetoxymethylpyridine 9-2. A second N-oxidation with m-CPBA and treatment with TMSCN and diethylcarbamyl chloride as described in Wilmer K. Fife, J. Org. Chem. 1983, 48, 1375-1377 and Sheng-Tung Huang and Dana M. Gordon, Tetrahedron Lett. 1998, 39, 9335 introduces a nitrile at the 6-position of the pyridine. This intermediate can be converted into the hydroxylmethyl ester 9-4 through treatment first with K₂CO₃/MeOH and then H⁺/MeOH. Sequential oxidation as laid out in Scheme 2, for instance Swern oxidation followed by treatment with sodium chlorate, followed by coupling to the secondary functionalized amine and cyclization under Mitsunobu conditions can afford the desired bicycle 9-7. The ester can then be converted to amides by heating with the appropriate amines. The benzyl group can then be removed (e.g., by hydrogenolysis) to give the desired inhibitor 9-8 and the acid 9-9 as side product.

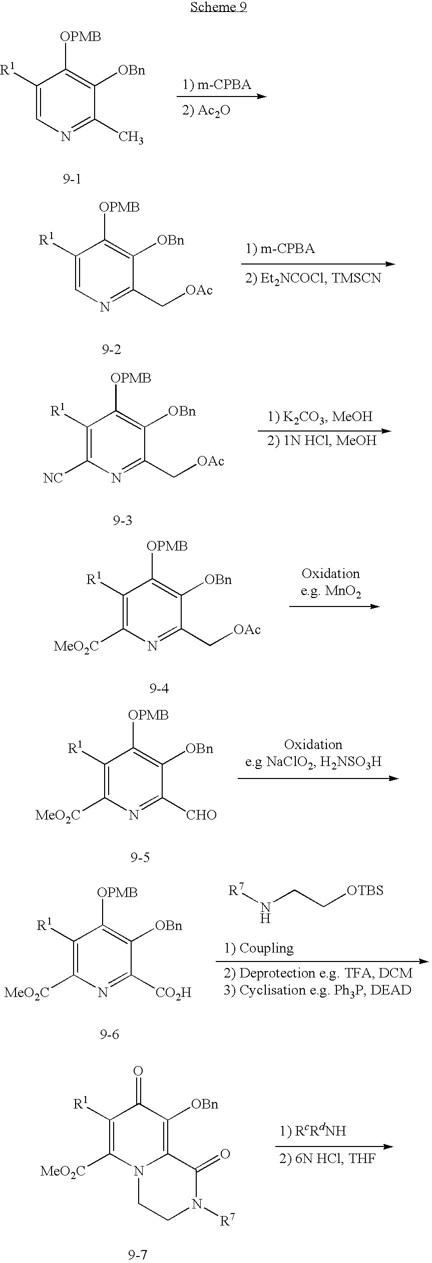

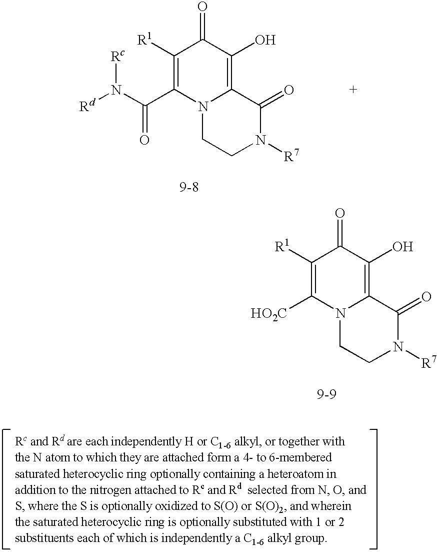

A method to introduce substituents onto the pyrazine ring is depicted in Scheme 10 whereby the functionalized carboxylic acid 2-4 is coupled with an amine 10-2 bearing an α,β-unsaturated ester. This amine 10-2 can be prepared as described in Tetrahedron 1997, 53 (32), 11126 by reacting amine 10-1 with ethyl 4-bromocrotonate in the presence of KF/celite. This amine can be coupled to the acid 2-4 using, for example, PyBOP to yield the desired amide 10-3. Treatment of this material with mineral acid (e.g., aqueous HCl in THF) results in cyclization to 10-4 with concurrent loss of the para-methoxybenzyl protecting group. Removal of the other protecting group (e.g. by hydrogenation) yields the desired ester 10-5 together with some carboxylic acid 10-6 as a result of hydrolysis.

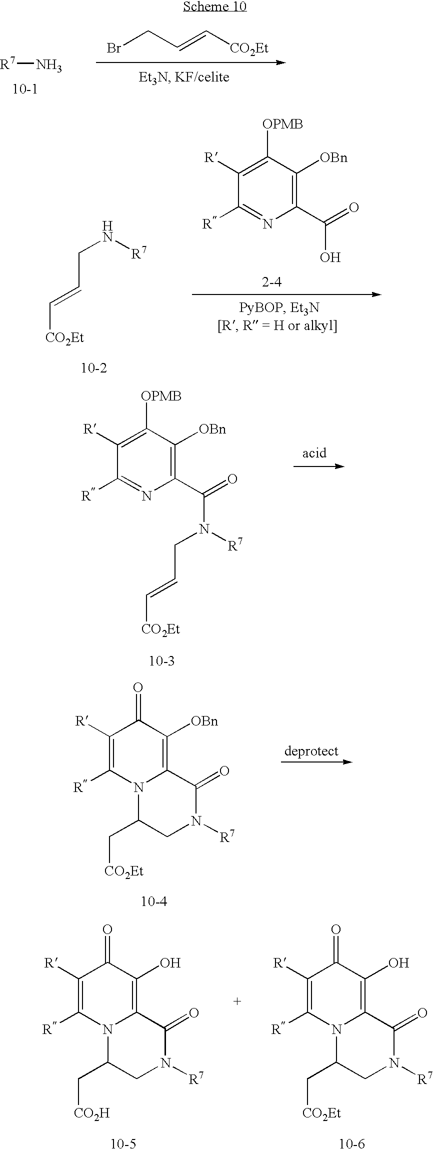

Alternatively the ester 10-4 can be converted into amides such as 11-2 as shown in Scheme 11, by hydrolysis of the ester 10-4 to the acid 11-1 by contacting 10-4 with an inorganic base (e.g., KOH in methanol-water at elevated temperature), followed by coupling the acid to an amine using a coupling reagent (e.g., PyBOP in the presence of triethylamine). Deprotection yields the desired compound of the invention 11-2.

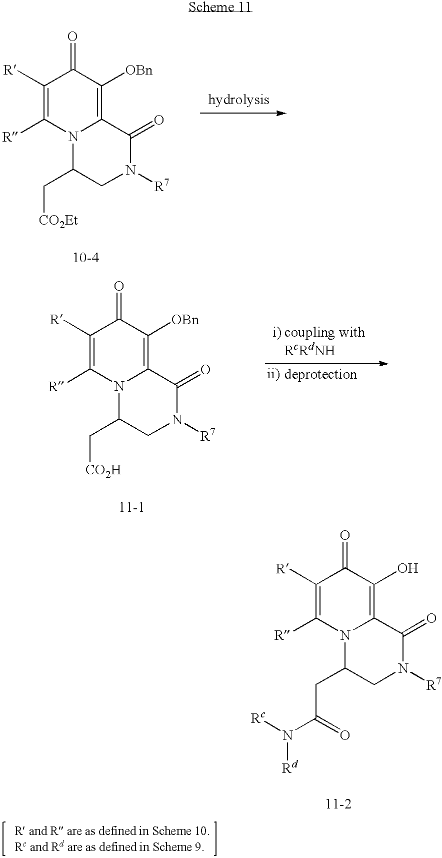

The ester 10-4 can also be transformed into amine 12-3 as depicted in Scheme 12 by reducing the ester to an alcohol 12-1 and subsequently oxidizing the alcohol to aldehyde 12-2, and then performing a reductive amination. Suitable methods to reduce an ester to an alcohol include treatment with LiAlH₄ and other reducing agents, such as those described in M. Hudlicky, Reductions in Organic Chemistry, American Chemical Society, Washington, 1996. The alcohol 12-1 can be oxidized to the corresponding aldehyde by the Swern method or by other methods such as those described in M. Hudlicky, Oxidations in Organic Chemistry, American Chemical Society, Washington, 1990. The reductive amination can be conducted using sodium cyanoborohydride and other agents and methods, such as those described in R. O. Hutchins in Comprehensive Organic Synthesis, edited by B. M. Trost, Pergamon Press, Vol. 8, 1993, p. 25 and E. W. Baxter and A. B. Reitz, Organic Reactions, edited by L. E. Overman, Vol. 59, John Wiley, 2002, p. 1. The desired compounds of the invention 12-3 can then be obtained from the aminated intermediate by deprotection (e.g., by hydrogenation such as H₂ with Pd/C) of the hydroxy group.

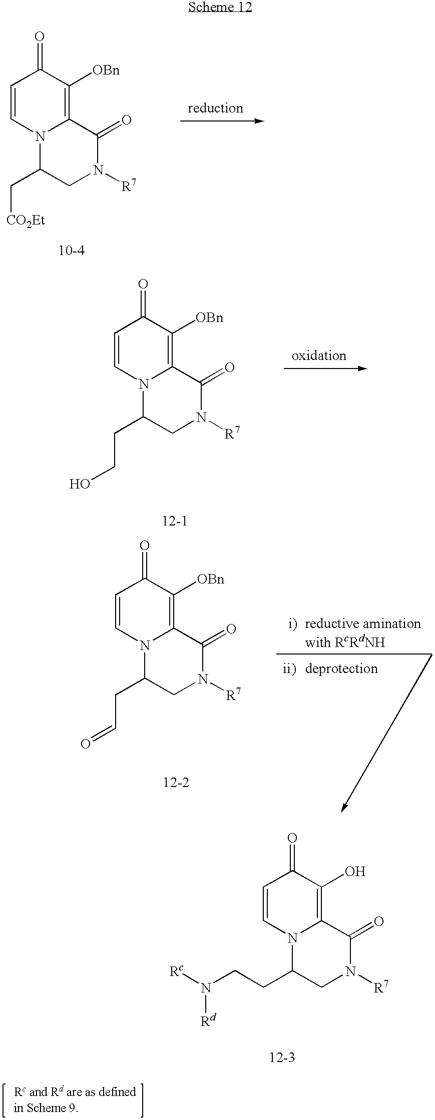

Tricyclic ring systems can be synthesized in the manner shown in Schemes 13 to 15. The tricyclic framework can be prepared from unsaturated amino acid 13-1 wherein the amine group can be readily protected with an amine protective group such as Boc as shown in Scheme 13 (other suitable amine protective groups are described in T. W. Greene and P. G. M. Wuts, Protective Groups in Organic Synthesis, 3^{rd} Edition, Wiley-Interscience, 1999; and P. J. Kocienski, Protecting Groups, Thieme, 1994) and the protected 13-1 converted into primary amide 13-2. The primary amide can then be dehydrated by treatment with a suitable dehydrating agent (e.g., triflic anhydride and a base such as triethylamine) to afford nitrile 13-3, which can be alkylated with a suitable alkylating agent (e.g., Me₂SO₄ in the presence of NaH) to afford 13-4. The alkylated nitrile 13-4 can then be reacted with hydroxylamine (e.g., in an alcohol such as isopropyl alcohol at elevated temperature such as 55-65° C.) and the resulting amidoxime intermediate can be then treated with dimethyl acetylenedicarboxylate to form adduct 13-5. This adduct can be thermally cyclized as described in J. Heterocyclic Chem. 1979, 16: p. 1423 (e.g., in xylene at 120-160° C.) to yield the required pyrimidine core, which can then be reacted with benzoic anhydride (e.g., with DMAP and pyridine) to protect the 5-hydroxyl group giving 13-6. Bromination of the terminal olefin using a suitable brominating agent (e.g., NBS) results in cyclization to the bicycle 13-7. Treatment of the bromide with sodium azide followed by hydrogenation results in ring closure to the tricyclic framework 13-8. The secondary amide can then be alkylated to afford 13-9 which can then be treated with a suitable amine deprotecting agent (e.g., aqueous TFA or HCl) to afford the desired compound 13-10. The diastereomers can be separated (e.g., by chiral chromatography) at the stage of final compounds or during the synthetic route.

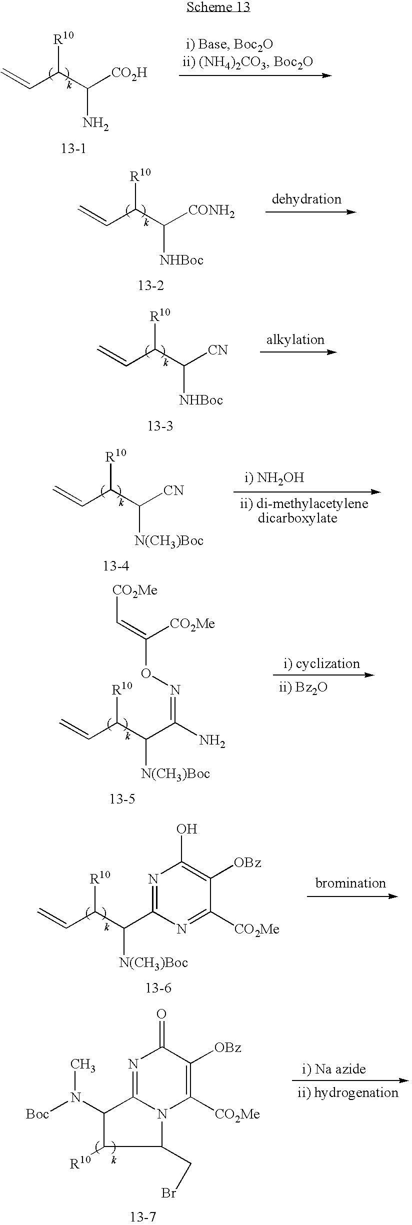

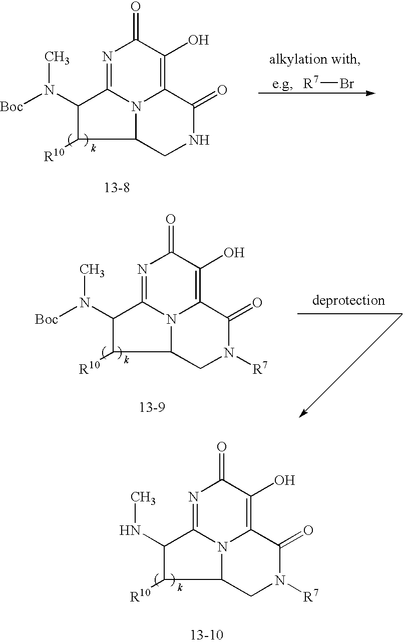

Occasionally the alkylation can be driven to occur twice (e.g., using an alkylating agent and NaH in the presence of 18-crown-6), which will afford, after deprotection of the amine group, compounds such as 14-1, as depicted in Scheme 14.

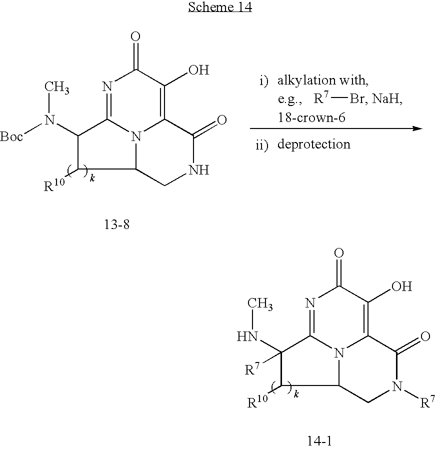

The pendant amino group in 13-10 can be functionalized in the manner depicted in Scheme 15 to give dialkyl amines and amides. For example, reductive amination of 13-10 with a suitable aldehyde using a suitable reducing agent such as sodium cyanoborohydride will afford amine 15-1. Alternatively, the free amine can be reacted with an acyl chloride such as methyl chlorooxoacetate to form amide 15-2 which can then be further functionalized further by reaction with an amine to form oxalamide 15-3.

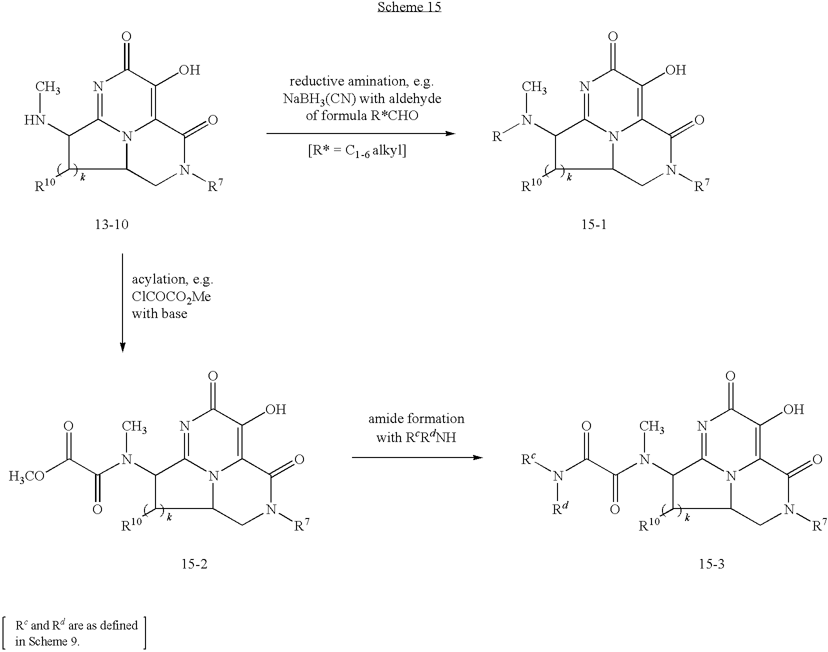

The following examples serve only to illustrate the invention and its practice. The examples are not to be construed as limitations on the scope or spirit of the invention.

### EXAMPLE 1

### 2-(4-Fluorobenzyl)-9-hydroxy-3,4-dihydro-2H-pyrido[1,2-α]pyrazine-1,8-dione

### Step 1

### 3-(Benzyloxy)-4-[(4-methoxybenzyl)oxy]-2-methylpyridine (A1)

DEAD (1.5 equivalent) was added dropwise over 10 minutes to a stirred solution of 3-(benzyloxy)-2-methylpyridin-4-ol (1.0 equivalent), 4-methoxybenzylalcohol (1.3 equivalents) and triphenylphosphine (1.5 equivalents) in THF at room temperature. The mixture was stirred overnight and then the solvent was removed under reduced pressure. The resulting mixture was triturated with EtOAc and hexanes, and filtered. The solution was concentrated under reduced pressure and then purified by column chromatography on silica eluting with 100% EtOAc to yield the desired pyridine A1. ¹H NMR (400 MHz, CDCl₃) δ 8.15 (1H, d, J=5.6 Hz), 7.72-7.30 (7H, m), 6.96 (2H, d, J=7.8 Hz), 6.83 (1H, d, J=5.6 Hz), 5.15 (2H, s), 4.98 (2H, s), 3.74 (3H, s), 2.45 (3H, s). MS (ES) C₂₁H₂₁NO₃ requires: 335. found: 336 (M+H⁺).

### Step 2

### {3-(Benzyloxy)-4-[(4-methoxybenzyl)oxy]pyridin-2-yl}methanol (A2)

mCPBA (2.0 equivalents) was added portionwise over 15 minutes to a stirred solution of the pyridine A1 (1.0 equivalent) in DCM at 0° C. The reaction was stirred overnight, gradually warming to room temperature. The reaction mixture was then diluted with DCM and washed with 1 M NaOH solution (3 times), then brine and dried (Na₂SO₄). The desired pyridine-N-oxide was used without further purification. MS (ES) C₂₁H₂₁NO₄ requires: 351. found: 352 (M+H⁺). The residue (1 equivalent) was taken up in excess Ac₂O, and the resulting mixture was heated at 130° C. for 90 minutes. After cooling to room temperature, the mixture was concentrated under reduced pressure and was then taken up in DCM. The solution was washed with saturated NaHCO₃ solution and brine, and then dried (Na₂SO₄) prior to concentrating under reduced pressure. MS(ES) C₂₃H₂₃NO₅ requires: 393. found: 394 (M+H⁺). The acetoxy derivative was taken up in MeOH and was treated with K₂CO₃ (1.5 equivalents). The mixture was stirred for 90 minutes and was then quenched by the addition of 6 M HCl solution. The MeOH was removed under reduced pressure and then more H₂O was added. The organics were extracted with DCM, and these DCM extracts were washed with brine and dried (Na₂SO₄). After concentrating under reduced pressure the desired alcohol A2 was obtained. ¹H NMR (400 MHz, CDCl₃) δ 8.19 (1H, d, J=5.6 Hz), 7.73-7.30 (10H, m), 6.89 (1H, d, J=5.6 Hz), 5.23 (2H, s), 5.07 (2H, s), 4.67 (2H, s). MS(ES) C₂₁H₂₁NO₄ requires: 351. found: 352 (M+H⁺).

### Step 3

### 3-(Benzyloxy)-4-[(4-methoxybenzyl)oxy]pyridine-2-carbaldehyde (A3)

Anhydrous DMSO (2.4 equivalents) was added dropwise over 10 minutes to a stirred solution of oxalyl chloride (1.2 equivalents) in dry DCM at −78° C. under N₂. The resulting mixture was then stirred at this temperature for 5 minutes and a solution of the above alcohol A2 (1 equivalent) in DCM was added dropwise over 10 minutes. After stirring for a further 30 minutes at −78° C., Et₃N (4.0 equivalents) was added dropwise over 5 minutes, the mixture was then stirred for 10 minutes and after the cooling bath was removed and the reaction was warmed to room temperature and stirred for an hour. After diluting with DCM, the mixture was washed with H₂O and then brine, dried (Na₂SO₄) and concentrated under reduced pressure. The residue was purified by column chromatography on silica eluting with 80% EtOAc/petroleum ether to yield the desired aldehyde A3. ¹H NMR (400 MHz, CDCl₃) δ 10.28 (1H, s), 8.44 (1H, d, J=5.6 Hz), 7.45-7.30 (7H, m), 7.11 (1H, d, J=5.6 Hz), 6.96 (2H, d, J=7.8 Hz), 5.18 (2H, s), 5.15 (2H, s), 3.88 (3H, s). MS (ES) C₂₁H₁₉NO₄ requires: 349. found: 368 (M+H₂O+H⁺).

### Step 4

### 3-(Benzyloxy)-4-[(4-methoxybenzyl)oxy]pyridine-2-carboxylic acid (A4)

Sulfamic acid (1.4 equivalents) and then sodium chlorite (1.1 equivalents) were added sequentially to a stirred solution of the aldehyde A3 (1.0 equivalents) in acetone and water. The resulting mixture was stirred at room temperature for 30 minutes and then the acetone was removed under reduced pressure. The organics were extracted with DCM, and then the DCM extracts were washed with brine. The extracts were dried (Na₂SO₄) and concentrated under reduced pressure to yield the desired acid A4. ¹H NMR (400 MHz, d₆-DMSO) δ 8.25 (1H, d, J=5.6 Hz), 7.48-7.27 (8H, m), 6.97 (2H, d, J=7.8 Hz), 5.24 (2H, s), 5.05 (2H, s), 3.78 (3H, s). MS(ES) C₂₁H₁₉NO₅ requires: 365. found: 366 (M+H⁺).

### Step 5

### 3-(Benzyloxy)-N-(2-{[tert-butyl(dimethyl)silyl]oxy}ethyl)-N-(4-fluorobenzyl)-4-[(4-ethoxybenzyl)oxy]pyridine-2-carboxamide (A5)

PyBOP (1.2 equivalents) was added to a stirred solution of the acid A4 (1.0 equivalent), (2-{[tert-butyl(dimethyl)silyl]oxy}ethyl)(4-fluorobenzyl)amine (1.2 equivalents) [Prepared from 4-fluorobenzylamine and 2-{[tert-butyl(dimethyl)silyl]-oxy}ethanal with NaBH₄ in MeOH] and Et₃N (1.5 equivalents) in DCM and the mixture was stirred at room temperature overnight. The reaction was diluted with DCM and washed sequentially with 0.5 N HCl solution, saturated NaHCO₃ solution and brine and then dried (Na₂SO₄). The resulting solution was concentrated under reduced pressure and then purified by column chromatography on silica eluting with 50-60% EtOAc/petroleum ether to yield the desired amide A5. MS (ES) C₃₆H₄₃FN₂O₅Si requires: 630. found: 631 (M+H⁺).

### Step 6

### 3-(Benzyloxy)-N-(4-fluorobenzyl)-N-(2-hydroxyethyl)-4-oxo-1,4-dihydropyridine-2-carboxamide (A6)

The amide A5 (1 equivalent) was taken up in THF and treated with 3.5 N HCl solution (7 equivalents). The resulting solution was stirred overnight and then was neutralized with solid NaOH. The THF was removed under reduced pressure and the organics were then extracted with DCM. The combined organic extracts were dried and concentrated under reduced pressure. The residue purified by column chromatography on silica eluting with 10-20% MeOH/DCM to yield the desired alcohol A6. MS(ES) C₂₂H₂₁FN₂O₄ requires: 396. found: 397 (M+H⁺).

### Step 7

### 9-(Benzyloxy)-2-(4-fluorobenzyl)-3,4-dihydro-2H-pyrido[1,2-α]pyrazine-1,8-dione (A7)

A mixture of the above alcohol A6 (1 equivalent), thionyl chloride (5 equivalents), and pyridine (10 equivalents) in CHCl₃ were heated at reflux for 8 hours. Further thionyl chloride (3.5 equivalents), and pyridine (10 equivalents) were added and heating was continued for a further 2 hours. The reaction was cooled to room temperature and was diluted with DCM. The mixture was washed with 1 N NaOH solution, H₂O and brine. After drying (Na₂SO₄) the mixture was concentrated under reduced pressure and purified by column chromatography on silica eluting with 10% MeOH/DCM to yield the desired bicycle A7. ¹H NMR (300 MHz, CDCl₃) δ 7.66 (2H, d, J=7.4 Hz), 7.45-7.27 (5H, m), 7.11 (1H, d, J=7.4 Hz), 7.07 (2H, t, J=8.4 Hz), 6.44 (1H, d, J=7.4 Hz), 5.37 (2H, s), 4.66 (2H, s), 3.88 (2H, t, J=5.3 Hz), 3.45 (2H, t, J=5.3 Hz). MS (ES) C₂₂H₁₉FN₂O₃ requires: 378. found: 379 (M+H⁺).

### Step 8

### 2-(4-Fluorobenzyl)-9-hydroxy-3,4-dihydro-2H-pyrido[1,2-α]pyrazine-1,8-dione (A8)

10% Pd on carbon was added to a stirred solution of the bicycle A7 (1 equivalent) in MeOH containing 1 M HCl solution (1.5 equivalents) and then after degassing the reaction vessel an H₂ atmosphere was introduced and the reaction was stirred for 90 minutes. The catalyst was filtered off through celite and the filter pad washed well with MeOH. The organics were concentrated under reduced pressure and the residue was purified by reverse phase HPLC to yield the desired bicycle A8. ¹H NMR (400 MHz, d₆-DMSO) δ 7.88 (1H, d, J=7.0 Hz), 7.45 (2H, dd, J=8.5, 5.5 Hz), 7.19 (2H, t, J=8.5 Hz), 6.57 (1H, d, J=7.0 Hz), 4.73 (2H, s), 4.33 (2H, t, J=5.5 Hz), 3.75 (2H, t, J=5.5 Hz). MS(ES) C₁₅H₁₃FN₂O₃ requires: 288. found: 289 (M+H⁺).

### EXAMPLE 2

### 2-(4-Fluorobenzyl)-9-hydroxy-7-pyridin-3-yl-3,4-dihydro-2H-pyrido[1,2-α]pyrazine-1,8-dione

### Step 1

### 9-(Benzyloxy)-2-(4-fluorobenzyl)-7-iodo-3,4-dihydro-2H-pyrido[1,2-α]pyrazine-1,8-dione (B1)

To a solution of the bicycle A7 (1 equivalent) in MeOH at 70° C. was added N-iodo-succinimide (4 equivalents) and then mCPBA (4 equivalent). The mixture was then heated at 75° C. for 3 hours and was subsequently concentrated under reduced pressure. The residue was taken up in DCM and washed with sodium sulfite solution and 0.5 N NaOH solution. The mixture was dried (Na₂SO₄) and concentrated under reduced pressure to yield the crude iodide B1. ¹H NMR (400 MHz, d₆-DMSO) δ 8.46 (1H, s), 7.53 (2H, d, J=6.6 Hz), 7.44-7.27 (5H, m), 7.19 (2H, t, J=8.9 Hz), 5.09 (2H, s), 4.67 (2H, s), 4.18 (2H, t, J=5.5 Hz), 3.13 (2H, t, J=5.5 Hz). MS(ES) C₂₂H₁₈FIN₂O₃ requires: 504. found: 505 (M+H⁺).

### Step 2

### 2-(4-Fluorobenzyl)-9-hydroxy-7-pyridin-3-yl-3,4-dihydro-2H-pyrido[1,2-α]pyrazine-1,8-dione (B2)

A mixture of the iodide B1 (1 equivalent) and 3-pyridyltributylstannane (3 equivalents) and Pd(PPh₃)₄ (10 mol %) in DMF was heated at 100° C. for 2 hours under N₂. The solvent was removed under reduced pressure whilst azeotroping with xylene. MS (ES) C₂₇H₂₂FN₃O₃ requires: 455. found: 456 (M+H⁺). The residue was taken up in THF and treated with 6 N HCl; this mixture was heated at 60° C. for 4 hours and was subsequently freeze dried and purified by reverse phase HPLC to yield the desired pyridine B2. ¹H NMR (300 MHz, d₆-DMSO) δ 9.39 (1H, s), 8.78 (1H, d, J=6.0 Hz), 8.38 (1H, s), 7.98 (1H, t, J=6.2 Hz), 7.47 (2H, dd, J=8.5, 5.5 Hz), 7.22 (2H, t, J=8.5 Hz), 6.57 (1H, d, J=7.0 Hz), 4.76 (2H, s), 4.33 (2H, t, J=5.5 Hz), 3.75 (2H, t, J=5.5 Hz). MS (ES) C₂₀H₁₆FN₃O₃ requires: 365. found: 366 (M+H⁺).

### EXAMPLE 3

### 7-Acetyl-2-(4-fluorobenzyl)-9-hydroxy-3,4-dihydro-2H-pyrido[1,2-α]pyrazine-1,8-dione

### Step 1

### 7-Acetyl-9-(benzyloxy)-2-(4-fluorobenzyl)-3,4-dihydro-2H-pyrido[1,2-α]pyrazine-1,8-dione (C1)

The iodide B1 (1 equivalent) was cross-coupled with 2-ethoxyvinyltributyl stannane as described in Example 2 Step 1. The crude residue, obtained after azeotroping with xylene, was taken up in THF and treated with 0.5 M HCl at room temperature for 30 minutes. The solution was neutralized with 1 N NaOH solution and extracted with DCM. The combined organic extracts were dried (Na₂SO₄) and concentrated under reduced pressure. The residue was then purified by column chromatography on silica eluting with 100% EtOAc to yield the desired ketone C1. ¹H NMR (400 MHz, CDCl₃) δ 8.02 (1H, s), 7.77-7.01 (9H, m), 5.43 (2H, s), 4.73 (2H, s), 4.04 (2H, t, J=5.5 Hz), 3.52 (2H, t, J=5.5 Hz), 2.77

(3H, s). MS (ES) C₂₄H₂₁FN₂O₄ requires: 420. found: 421 (M+H⁺).

### Step 2

### 7-Acetyl-2-(4-fluorobenzyl)-9-hydroxy-3,4-dihydro-2H-pyrido[1,2-α]pyrazine-1,8-dione (C2)

The ketone C1 (1 equivalent) was taken up in THF and treated with 6 N HCl; this mixture was heated at 60° C. for 4 hours and was then concentrated under reduced pressure and purified by reverse phase HPLC to yield the desired bicycle C2. ¹H NMR (300 MHz, d₆-DMSO) δ 12.40 (1H, br. s), 8.17 (1H, s), 7.44 (2H, dd, J=8.7, 5.8 Hz), 7.18 (2H, t, J=8.7 Hz), 4.72 (2H, s), 4.33 (2H, t, J=5.5 Hz), 3.72 (2H, t, J=5.5 Hz), 2.57 (3H, s). MS(ES) C₁₇H₁₅FN₂O₄ requires: 330. found: 331 (M+H⁺).

### EXAMPLE 4

### 2-(4-Fluorobenzyl)-9-hydroxy-7-(1-hydroxyethyl)-3,4-dihydro-2H-pyrido[1,2-α]pyrazine-1,8-dione

### Step 1

### 9-(Benzyloxy)-2-(4-fluorobenzyl)-7-(1-hydroxyethyl)-3,4-dihydro-2H-pyrido[1,2-a]pyrazine-1,8-dione (D1)

Sodium borohydride (1 equivalent) was added to a stirred solution of the C1 (1 equivalent) in EtOH and the resulting mixture was stirred at room temperature for 45 minutes. The reaction was quenched with NH₄Cl solution was added and the solvent was removed under reduced pressure. H₂O was added and then the organics were extracted with DCM. The organic extracts were dried (Na₂SO₄), and concentrated under reduced pressure to yield the alcohol D1. MS(ES) C₂₄H₂₃N₂O₄F requires: 422. found: 423 (M+H⁺).

### Step 2

### 2-(4-Fluorobenzyl)-9-hydroxy-7-(1-hydroxyethyl)-3,4-dihydro-2H-pyrido[1,2-α]pyrazine-1,8-dione (D2)

The bicycle D1 was deprotected in accordance with the procedure described in Example 1 Step 8 to yield D2 after reverse phase HPLC. ¹H NMR (300 MHz, d₆-DMSO) δ 7.87 (1H, s), 7.47 (2H, dd, J=8.6, 5.5 Hz), 7.26 (2H, t, J=8.6 Hz), 4.92 (1H, q, J=6.4 Hz), 4.87 (2H, s), 4.42 (2H, t, J=5.5 Hz), 3.77 (2H, t, J=5.5 Hz), 1.35 (3H, d, J=6.4 Hz). MS (ES) C₁₇H₁₇N₂O₄F requires: 332. found: 333 (M++).

### EXAMPLE 5

### 2-(4-Fluorobenzyl)-9-hydroxy-7-[1-(methylamino)ethyl]-3,4-dihydro-2H-pyrido[1,2-α]pyrazine-1,8-dione

### Step 1

### 9-(Benzyloxy)-2-(4-fluorobenzyl)-7-[1-(methylamino)ethyl]-3,4-dihydro-2H-pyrido[1,2-α]pyrazine-1,8-dione (E1)

Sodium cyanoborohydride (6 equivalents) was added to a stirred solution of methylamine. HCl (10 equivalents) and the ketone C1 (1 equivalent) in MeOH and the mixture was stirred at room temperature overnight. The mixture was quenched by the addition of NH₄Cl solution and 1 M NaOH. The MeOH was removed under reduced pressure and the organics were then extracted with DCM, dried (Na₂SO₄) and concentrated under reduced pressure. MS(ES) C₂₅H₂₆FN₃O₃ requires: 435, found: 436 (M+H⁺).

### Step 2

### 2-(4-Fluorobenzyl)-9-hydroxy-7-[1-(methylamino)ethyl]-3,4-dihydro-2H-pyrido[1,2-α]pyrazine-1,8-dione (E2)

The bicycle E1 was deprotected as described in Example 1 step 8 to yield E2, as the TFA salt, after reverse phase HPLC. ¹H NMR (300 MHz, d₆-DMSO) δ 12.25 (1H, br. s), 8.85 (1H, br. s), 8.70 (1H, br. s), 7.87 (1H, s), 7.47 (2H, dd, J=8.8, 5.7 Hz), 7.26 (2H, t, J=8.8 Hz), 4.73 (2H, s), 4.38-4.24 (3H, m), 3.77 (2H, t, J=5.5 Hz), 2.44 (3H, t, J=4.9 Hz), 1.55 (3H, d, J=6.8 Hz). MS(ES) C₁₈H₂₀N₃O₃F requires: 345. found: 346 (M+H⁺).

### EXAMPLE 6

### N-{1-[2-(4-Fluorobenzyl)-9-hydroxy-1,8-dioxo-1,3,4,8-tetrahydro-2H-pyrido[1,2-α]pyrazin-7-yl]ethyl}-N-methylacetamide

### Step 1

### N-{1-[2-(4-Fluorobenzyl)-9-hydroxy-1,8-dioxo-1,3,4,8-tetrahydro-2H-pyrido[1,2-α]pyrazin-7-yl]ethyl}-N-methylacetamide (F1)

Ac₂O (3 equivalents) was added to a stirred solution of the crude amine E1 (1 equivalent) and Et₃N (3 equivalents) in DCM and the resulting mixture was stirred at room temperature for 1 hour. More DCM was added and the mixture was washed with saturated aqueous NaHCO₃ solution, and brine. The solvent was removed under reduced pressure and the crude residue was purified by column chromatography on silica eluting with 3-6% MeOH/DCM to yield the desired acetamide F1. MS(ES) C₂₇H₂₈FN₃O₄ requires: 477. found: 478 (M+H⁺). The intermediate was deprotected as described in Example 1 step 8 to yield F2 after reverse phase HPLC. ¹H NMR (400 MHz, d₆-DMSO) Major Rotamer: δ 7.83 (1H, s), 7.47-7.35 (2H, m), 7.19 (2H, t, J=8.6 Hz), 5.18 (1H, q, J=7.0 Hz), 4.80-4.65 (2H, m), 4.30-4.18 (2H, m), 3.78-3.65 (2H, m), 2.78 (3H, s), 2.29 (3H, s), 1.38 (3H, d, J=7.0 Hz). MS(ES) C₂₀H₂₂N₃O₄F requires: 387. found: 388 (M+H⁺).

### EXAMPLE 7

### 2-(4-Fluorobenzyl)-9-hydroxy-6-methyl-3,4,6,7-tetrahydro-2H-pyrazino[1,2-c]pyrimidine-1,8-dione and 2-(4-Fluorobenzyl)-9-hydroxy-6-methyl-3,4-dihydro-2H-pyrazino[1,2-c]pyrimidine-1,8-dione

### Step 1

### 5-(Benzyloxy)-6-hydroxy-2-methylpyrimidine-4-carboxylic acid (G1)

A solution of ethyl 5-(benzyloxy)-6-hydroxy-2-methylpyrimidine-4-carboxylate (1 equivalent) [Inorganic Chem. 2001, 40, 6746] in MeOH was treated with KOH (3.4 equivalents) and the mixture was heated at reflux of 90 minutes. The reaction was quenched by the addition of 6 M HCl solution and the solvent was removed under reduced pressure. The organics were dissolved in 5% MeOH/DCM and were dried (Na₂SO₄), filtered and concentrated under reduced pressure to yield the acid G1. ¹H NMR (400 MHz, d₆-DMSO) δ 7.45-7.30 (5H, m), 5.13 (2H, s), 2.29 (3H, s). MS(ES) C₁₃H₁₂N₂O₄ requires: 260. found: 261 (M+H⁺).

### Step 2

### 5-(Benzyloxy)-N-(4-fluorobenzyl)-6-hydroxy-N-(2-hydroxyethyl)-2-methylpyrimidine-4-carboxamide (G2)

The acid G1 was coupled with (2-{[tert-butyl(dimethyl)silyl]-oxy}ethyl)(4-fluorobenzyl)amine via the procedure described in Example 1 Step 5 to yield after purification by column chromatography on silica eluting with 100% EtOAc to yield the desired amide. MS (ES) C₂₈H₃₆FN₃O₄Si requires: 525. found: 526 (M+H⁺). The intermediate was taken up in THF and treated with 1 M HCl (1.5 equivalents). After stirring at room temperature for 1 hour the reaction was quenched by the addition of 1 M NaOH solution. The organics were extracted with DCM, dried (Na₂SO₄) and concentrated under reduced pressure. Column chromatography on silica eluting with 5% MeOH/DCM yielded the desired alcohol G2. ¹H NMR (400 MHz, d₆-DMSO) Major Rotamer: δ 7.48-7.30 (7H, m), 6.95 (2H, t, J=8.5 Hz), 5.15 (2H, s), 4.73 (2H, s), 3.48-3.37 (2H, m), 3.15-3.00 (2H, m) 2.29 (3H, s). MS (ES) C₂₂H₂₂FN₃O₄ requires: 411. found: 412 (M+H⁺).

### Step 3

### 9-(Benzyloxy)-2-(4-fluorobenzyl)-6-methyl-3,4-dihydro-2H-pyrazino[1,2-c]pyrimidine-1,8-dione (G3)

DEAD (1.5 equivalent) was added dropwise over 10 minutes to a stirred solution of the alcohol G2 (1.0 equivalent) and triphenylphosphine (1.5 equivalents) in DCM at room temperature. The mixture was stirred for 90 minutes and then the solvent was removed under reduced pressure whilst dry loading onto silica. The desired bicycle was purified by column chromatography on silica eluting with 5% MeOH/DCM to yield G3. ¹H NMR (400 MHz, d₆-DMSO) δ 7.51 (2H, d, J=6.6 Hz), 7.43-7.31 (5H, m), 7.18 (2H, t, J=8.8 Hz), 5.09 (2H, s), 4.67 (2H, s), 4.07-3.97 (2H, m), 3.60-3.52 (2H, m) 2.37 (3H, s). MS (ES) C₂₂H₂₀FN₃O₃ requires: 393. found: 394 (M+H⁺).

### Step 4

### 2-(4-Fluorobenzyl)-9-hydroxy-6-methyl-3,4-dihydro-2H-pyrazino[1,2-c]pyrimidine-1,8-dione (G5) and 2-(4-fluorobenzyl)-9-hydroxy-6-methyl-3,4,6,7-tetrahydro-2H-pyrazino[1,2-c]pyrimidine-1,8-dione (G4)

The bicycle G3 was deprotected in the manner described in Example 1 Step 8, except that acid was not present, to yield after reverse phase HPLC, eluted first G5 and then G4.

Spectra for G5: ¹H NMR (400 MHz, d₆-DMSO) δ 7.44 (2H, dd, J=5.7, 8.6 Hz), 7.18 (2H, t, J=8.6 Hz), 4.73 (2H, s), 4.32 (2H, t, J=5.5 Hz), 3.68 (2H, t, J=5.5 Hz), 2.45 (3H, s). MS (ES) C₁₅H₁₄FN₃O₃ requires: 303. found: 304 (M+H⁺).

Spectra for G4: ¹H NMR (400 MHz, d₆-DMSO) δ 11.08 (1H, br. s), 8.09 (1H, s), 7.34 (2H, dd, J=5.7, 8.6 Hz), 7.16 (2H, t, J=8.6 Hz), 4.63 (1H, d, J=8.8 Hz), 4.58 (1H, d, J=8.8 Hz), 4.23 (1H, q, J=8.8 Hz), 3.56-3.32 (2H, m), 3.16-3.07 (1H, m), 2.78-2.66 (1H, m), 1.26 (3H, d, J=6.0 Hz). MS (ES) C₁₅H₁₆FN₃O₃ requires: 305. found: 306 (M+H⁺).

### EXAMPLE 8

### 2-(4-Fluorobenzyl)-9-hydroxy-6-(morpholin-4-ylmethyl)-3,4-dihydro-2H-pyrazino[1,2-c]pyrimidine-1,8-dione

### Step 1

### 2-(4-Fluorobenzyl)-9-hydroxy-6-(morpholin-4-ylmethyl)-3,4-dihydro-2H-pyrazino[1,2-c]pyrimidine-1,8-dione (H1)

A solution of the bicycle G3 (1 equivalent) and freshly recrystallized N-bromo-succinimide (1.4 equivalents) in DMF was treated with catalytic benzoyl peroxide and the mixture was heated at 70° C. for 40 minutes to yield the bromo-derivative. MS (ES) C₂₂H₁₉BrFN₃O₃ requires: 471, found: 472 (M+H⁺). Morpholine (10 equivalents) was added to the reaction mixture and the temperature was raised to 90° C. for 40 minutes. The solvent was removed under reduced pressure whilst azeotroping with xylene. MS (ES) C₂₆H₂₇FN₄O₄ requires: 478. found: 479 (M+H⁺). The crude residue was deprotected as described in Example 1 step 8 to yield the desired amine H1 after reverse phase HPLC purification. ¹H NMR (400 MHz, d₆-DMSO) δ 12.22 (1H, br. s), 7.47 (2H, dd, J=8.5, 5.7 Hz), 7.25 (2H, t, J=8.5 Hz), 4.78 (2H, s), 4.25-4.18 (4H, m), 3.84-3.60 (8H, m), 3.15-2.90 (2H, m). MS (ES) C₁₉H₂₁FN₄O₄ requires: 388. found: 389 (M+H⁺).

### EXAMPLE 9

### 7-Bromo-2-(4-fluorobenzyl)-9-hydroxy-6-methyl-3,4-dihydro-2H-pyrido[1,2-α]pyrazine-1,8-dione

### Step 1

### 7-Bromo-2-(4-fluorobenzyl)-9-hydroxy-6-methyl-3,4-dihydro-2H-pyrido[1,2-α]pyrazine-1,8-dione (I1)

To a solution of 9-(benzyloxy)-2-(4-fluorobenzyl)-6-methyl-3,4-dihydro-2H-pyrido[1,2-α]pyrazine-1,8-dione the bicycle 10 (1 equivalent) [Prepared in a similar manner to Example 1] in DCM was treated with bromine (2 equivalents) and stirred at room temperature for 24 hours. The reaction was concentrated under reduced pressure and the residue was purified by reverse phase HPLC to yield the desired bicycle I1. ¹H NMR (300 MHz, d₆-DMSO) δ 7.48 (1H, dd, J=8.6, 5.5 Hz), 7.26 (2H, t, J=8.6 Hz), 4.79 (2H, s), 4.30 (2H, t, J=5.4 Hz), 3.75 (2H, t, J=5.4 Hz), 2.61 (3H, s). MS(ES) C₁₆H₁₄BrFN₂O₃ requires: 380. found: 381 (M+H⁺).

### EXAMPLE 10

### (+/−) cis tert-Butyl[(2RS,8aRS)-7-(4-fluorobenzyl-5-hydroxy-4,6-dioxo-2,4,6,7,8,8a-hexahydro-1H-3,7,8b-triazaacenaphthylen-2-yl]methylcarbamate and (+/−) trans tert-Butyl[(2RS,8aRS)-7-(4-fluorobenzyl-5-hydroxy-4,6-dioxo-2,4,6,7,8,8a-hexahydro-1H-3,7,8b-triazaacenaphthylen-2-yl]methylcarbamate

### Step 1

### tert-Butyl[1-(aminocarbonyl)but-3-en-1-yl]carbamate (J1)

To a solution of 2-aminopent-4-enoic acid in 1,4-dioxane/water (1:2) were added KHCO₃ (1.1 equivalents) and Boc₂O (1 equivalent) and the suspension was stirred at room temperature. After 18 hours, the solvent was concentrated under reduced pressure and the residue dissolved in CHCl₃. The organic phase was washed with 1N HCl, dried (Na₂SO₄) and concentrated under reduced pressure. The residue in was dissolved in 1,4-dioxane/pyridine (10:1) and (NH₄)₂CO₃ (1.1 equivalents) and Boc₂O (1.1 equivalents) were added. The suspension was stirred at room temperature for 16 hours, EtOAc was added and the organic phase was washed with 1N HCl, dried (Na₂SO₄) and the filtrate concentrated under reduced pressure to give a white solid. ¹H-NMR (400 MHz, CDCl₃) δ: 7.74 (1H, br. s), 6.62 (1H, br. s), 6.51 (1H, br. s), 5.81-5.71 (1H, m), 5.21-5.13 (2H, m), 4.23 (1H, br. s), 2.58-2.45 (2H, m), 1.45 (9H, s). MS (ES) C₁₀H₁₈N₂O₃ requires: 214. found: 214 (M)⁺.

### Step 2

### tert-Butyl (1cyanobut-3-en-1-yl)carbamate (J2)

To a solution of tert-butyl[1-(aminocarbonyl)but-3-en-1-yl]carbamate (J1) in DCM at 0° C. were added Et₃N (2.2 equivalents) and Tf₂O (1.1 equivalents) and the reaction mixture was stirred at room temperature. After 2 hours, 1N HCl was added and the organic phase was separated and washed with sat. aq. NaHCO₃ solution and brine. The combined organic layer was dried (Na₂SO₄) and concentrated under reduced pressure. The residue was purified by column chromatography on silica gel eluting with 20% EtOAc/Petroleum ether to yield the desired nitrile. ¹H-NMR (400 MHz, CDCl₃) δ: 5.83-5.72 (1H, m), 5.27-5.23 (3H, m), 4.59 (1H, br. s), 2.56-2.47 (2H, m), 1.45 (9H, s). MS (ES) C₁₀H₁₆N₂O₂ requires: 196. found: 197 (M+H)⁺.

### Step 3

### tert-Butyl (1-cyanobut-3-en-1-yl)methylcarbamate (J3)

A solution of tert-butyl (1 cyanobut-3-en-1-yl)carbamate (J2) in THF and H₂O (0.2 equivalents) was added to NaH (2 equivalents) in THF. After 10 minutes Me₂SO₄ (1.8 equivalents) was added and the reaction mixture was stirred at room temperature for 1 hour, after which, ammonia, toluene and water were added and the organic phase was separated. The aqueous phase was extracted with toluene, and the combined organic layers were dried (Na₂SO₄) and concentrated under reduced pressure to yield the desired material. ¹H-NMR (400 MHz, CDCl₃) δ: 5.76-5.65 (1H, m), 5.25-5.15 (3H, m), 2.82 (3H, s), 2.67-2.57 (2H, m), 1.42 (9H, s). MS (ES) C₁₁H₁₈N₂O₂ requires: 210. found: 211 (M+H)⁺.

### Step 4

### Dimethyl 2-[1-amino-2-[(tert-butoxycarbonyl)(methyl)amino]pent-4-en-1-ylidene]oxy]but-2-enedioate (J4)

To a solution of tert-butyl (1-cyanobut-3-en-1-yl)methylcarbamate (J3) in i-PrOH was added NH₂OH (10 equivalents) and the solution was stirred at 60° C. for 16 hours. The solution was concentrated under reduced pressure and the residue dissolved in MeOH, dimethyl acetylenedicarboxylate (2.7 equivalents) was added and the solution was stirred at room temperature. After 3 hours the solvent was concentrated under reduced pressure and the residue purified by chromatography on silica gel eluting with 80% EtOAc/Petroleum ether to yield the desired material as a mixture of isomers. ¹H-NMR (400 MHz, CDCl₃) δ: 6.5 (0.5H, br. s); 6.18 (1.5H, s), 5.7-5.6 (2H, m), 5.15-5.0 (2H, m), 4.76-4.62 (1H, m), 3.81 (0.5H, s), 3.77 (0.5H, s), 3.75 (2H, s), 3.64 (2H, s), 3.59 (1H, s), 2.68 (1H, s), 2.63 (2H, s), 2.46-2.26 (2H, m), 1.39 (9H, s).

### Step 5

### Methyl 2-[[1-[(tert-butoxycarbonyl)(methyl)amino]but-3-en-1-yl]]-5,6-dihydroxypyrimidine-4-carboxylate (J5)

A solution of dimethyl 2-[1-amino-2-[(tert-butoxycarbonyl)(methyl)amino]pent-4-en-1-ylidene]oxy]but-2-enedioate (J4) in xylene was stirred at 140° C. After 5 hours the solvent was concentrated under reduced pressure and the residue was dissolved in EtOAc and washed with sat. aq. NaHCO₃ solution. The combined aqueous layers were acidified with 6N HCl and extracted with DCM. The combined DCM layers were dried (Na₂SO₄) and concentrated under reduced pressure to yield the desired pyrimidine. ¹H-NMR (400 MHz, d₆-DMSO) δ: 12.89 (1H, br. s), 10.35 (1H, s), 5.75 (1H, br. s), 5.15-5.03 (2.5H, m), 4.76 (0.5H, br. s), 3.85 (3H, s), 2.73 (3H, s), 2.73-2.68 (1H, m), 2.41-2.20 (1H, m), 1.39-1.24 (9H, m). MS (ES) C₁₆H₂₃N₃O₆ requires: 353. found: 354 (M+H)⁺.

### Step 6

### Methyl 5-(benzoyloxy)-2-[[1-[(tert-butoxycarbonyl)(methyl)amino]but-3-en-1-yl]]-6-hydroxypyrimidine-4-carboxylate (J6)

To a solution of methyl 2-[[1-[(tert-butoxycarbonyl)(methyl)amino]but-3-en-1-yl]]-5,6-dihydroxypyrimidine-4-carboxylate (J5) in DCM/pyridine (5:1) were added Bz₂O (1 equivalent) and DMAP (0.1 equivalents) and the solution was stirred at room temperature. After 18 hours the solution was concentrated under reduced pressure. The residue was dissolved in EtOAc, washed with sat. aq. NaHCO₃ solution and 1N HCl, dried (Na₂SO₄) and concentrated under reduced pressure to yield the desired benzoate. ¹H-NMR (300 MHz, d₆-DMSO) δ: 8.01 (2H, d, J=7.3 Hz), 7.92 (1H, t, J=7.5 Hz), 7.63 (1H, t, J=7.5 Hz), 5.81 (1H, br. s), 5.25-5.15 (2H, m), 4.80-4.55 (1H, m), 3.76 (3H, s), 2.85 (3H, s), 2.85-2.58 (2H, m), 1.48-1.27 (9H, m). MS (ES) C₂₃H₂₇N₃O₇ requires: 473. found: 474 (M+H)⁺.

### Step 7

### Methyl 3-(benzoyloxy)-6-(bromomethyl)-8-[(tert-butoxycarbonyl)(methyl)amino]2-oxo-2,6,7,8-tetrahydropyrrolo[1,2-α]pyrimidine-4-carboxylate (J7)

To a solution of methyl 5-(benzoyloxy)-2-[[1-[(tert-butoxycarbonyl)(methyl)amino]but-3-en-1-yl]]-6-hydroxypyrimidine-4-carboxylate (J6) in DMSO were added H₂O (2 equivalents) and NBS (2 equivalents) and the solution was stirred at room temperature. After 10 minutes H₂O was added and the mixture was extracted with EtOAc. The combined organic layers were dried (Na₂SO₄) and concentrated under reduced pressure. The product was purified by preparative RP-HPLC (using H₂O (0.1% TFA) and MeCN (0.1% TFA) as eluants, column: C18) and the two diastereoisomers were separated. The products were obtained after lyophilization of the pooled product fractions.

Diastereomer A: ¹H-NMR (300 MHz cryo, 330K, d₆-DMSO) δ: 8.07 (2H, d, J=7.4 Hz), 7.80-7.77 (1H, m), 7.63 (2H, t, J=7.7 Hz), 5.35-5.20 (1H, m), 4.88 (1H, br. s), 3.89 (3H, s), 3.91-3.78 (2H, m), 2.9 (3H, s), 2.71-2.5 (2H, m), 1.44 (9H, br. s). MS (ES) C₂₃H₂₆BrN₃O₇ requires: 536. found: 537 (M+H)⁺.

Diastereomer B: ¹H-NMR (500 MHz, 325K, d₆-DMSO) δ: 8.07 (2H, dd, J=8.2, 1.1 Hz), 7.77 (1H, t, J=7.6 Hz), 7.62 (2H, t, J=8.2 Hz), 5.53 (1H, t, J=9.7 Hz), 5.04-5.01 (1H, m), 3.88 (3H, s), 3.82 (1H, dd, J=11.4, 1.6 Hz), 3.75-3.68 (1H, m), 2.85-2.79 (4H, m), 2.12-2.06 (1H, m), 1.43 (9H, s). MS (ES) C₂₃H₂₆BrN₃O₇ requires: 536. found: 537 (M+H)⁺.

### Step 8

### tert-Butyl (5-hydroxy-4,6-dioxo-2,4,6,7,8,8a-hexahydro-1H-3,7,8b-triazaacenaphthylen-2-yl)methylcarbamate (J8)

Sodium azide (2 equivalents) was added to a solution of a mixture of the diastereomers of methyl 3-(benzoyloxy)-6-(bromomethyl)-8-[(tert-butoxycarbonyl) (methyl)amino]2-oxo-2,6,7,8-tetrahydropyrrolo[1,2-α]pyrimidine-4-carboxylate (J7) in DMF and the solution was stirred at room temperature. After 48 hours the solution was concentrated under reduced pressure. The residue was dissolved in MeOH and Pd/C (10%) was added and the reaction mixture was stirred at room temperature under an H₂ atmosphere. After 16 hours the suspension was filtered over celite and the filtrated was concentrated under reduced pressure. The product was purified by preparative RP-HPLC, using a gradient of H₂O (0.1% TFA) and MeCN (0.1% TFA) as eluants (column: C18) and the product was obtained after lyophilization of the desired fractions.

Two patterns of signals corresponding to two diastereoisomers 1:1: ¹H-NMR (500 MHz, 300K, d₆-DMSO) O: 8.75 (1H, s), 5.61-5.54 (0.2H, m), 5.21-4.97 (0.8H, m), 4.62 (1H, br. s), 4.28 (1H, br. s), 3.71-3.60 (1H, m), 3.49 (0.3H, t, J=12 Hz), 3.39 (0.7 Hz, t, J=12 Hz), 2.80 (2H, s), 2.70 (1H, s), 2.34 (0.5H, br. s), 2.19 (0.5H, br. s), 2.05-1.94 (0.5H, m), 1.43-1.28 (9H, m). MS (ES) C₁₅H₂₀N₄O₅ requires: 336. found: 337 (M+H)⁺.

### Step 9

### (+/−) cis tert-Butyl[7-(4-fluorobenzyl-5-hydroxy-4,6-dioxo-2,4,6,7,8,8a-hexahydro-1H-3,7,8b-triazaacenaphthylen-2-yl]methylcarbamate (J9) and (+/−) trans tert-Butyl [(2RS,8aRS)-7-(4-fluorobenzyl-5-hydroxy-4,6-dioxo-2,4,6,7,8,8a-hexahydro-1H-3,7,8b-triazaacenaphthylen-2-yl]methylcarbamate (J10)

To a suspension of KH (3 equivalents) in THF was added a solution of tert-butyl (5-hydroxy-4,6-dioxo-2,4,6,7,8,8a-hexahydro-1H-3,7,8b-triazaacenaphthylen-2-yl)methylcarbamate (J8) in DMF and the reaction mixture was stirred at room temperature. After 10 minutes para-fluorobenzyl bromide (2 equivalents) was added and the resulting solution was stirred at room temperature. After 16 hours AcOH was added and the reaction mixture was concentrated to dryness under reduced pressure. The product was purified by preparative RP-HPLC, separating the diastereoisomer by, using a gradient of water (0.1% TFA) and acetonitrile (0.1% TFA) as eluants (column: C18). The products were obtained after lyophilization of the desired fractions.

Diastereomer A, cis-isomer, (J9): More polar, first to be eluted: Two patterns of signal corresponding to two conformers: ¹H-NMR (600 MHz cryo, 300K, d₆-DMSO) δ: 7.39-7.37 (2H, m), 7.21-7.17 (2H, m), 5.60-5.57 (0.5H, m), 5.19 (0.5H, br. s), 4.81-4.77 (1H, m), 4.54 (1H, dd, J=22.5, 14.7 Hz), 4.35 (1H, br. s), 3.77-3.36 (4H, m), 2.76 (1.5H, s), 2.67 (1.5H, s), 2.53-2.49 (1H, m), 2.02-1.92 (1H, m), 1.43 (4.5H, s), 1.27 (4.5H, s). MS (ES) C₂₂H₂₅FN₄O₅ requires: 444. found: 445 (M+H)⁺.

Diastereomer B, trans-isomer (J10): Less polar, second to be eluted: ¹H-NMR (600 MHz cryo, 296K, DMSO) δ: 10.35 (1H, br. s), 7.38 (2H, br. s), 7.20 (2H, t, J=8.8 Hz), 5.18-5.01 (1H, m), 4.73-4.58 (2H, m), 3.70 (1H, dd, J=12.2, 3.5 Hz), 3.57 (1H, t, J=12.2 Hz), 2.77 (3H, s), 2.36-2.26 (1H, m), 2.23-2.12 (1H, m), 1.43-1.17 (9H, m). MS (ES) C₂₂H₂₅FN₄O₅ requires: 444. found: 445 (M+H)⁺.

### EXAMPLE 11

### 2,7-bis(4-Fluorobenzyl)-5-hydroxy-2-methylamino)8,8a-dihydro-1H-3,7,8b-triazaacenaphthylene-4,6(2H,7H)-dione trifluoroacetate salt (L1)

To a solution of tert-butyl (5-hydroxy-4,6-dioxo-2,4,6,7,8,8a-hexahydro-1H-3,7,8b-triazaacenaphthylen-2-yl)methylcarbamate (J8) in DMF were added NaH (6 equivalents) and 18-crown-6 (1 equivalent) and the reaction mixture was stirred at 40° C. After 15 minutes para-fluorobenzyl bromide (2 equivalents) was added and the suspension was stirred at 70° C. After 2 hours the reaction mixture was cooled to room temperature and concentrated under reduced pressure. The product was purified by preparative RP-HPLC, using a gradient of H₂O (0.1% TFA) and MeCN (0.1% TFA) as eluants (column: C18) and the desired fractions lyophilised to give tert-butyl[2,7-bis(4-fluorobenzyl)-5-hydroxy-4,6-dioxo-2,4,6,7,8,8a-hexahydro-1H-3,7,8b-triazaacenaphthylen-2-yl]methylcarbamate. To the resulting material in DCM was added TFA and the solution was stirred at room temperature. After 2 hours the reaction mixture was concentrated under reduced pressure. The product was purified by preparative RP-HPLC, using a gradient of H₂O (0.1% TFA) and MeCN (0.1% TFA) as eluants (column: C18) and the desired fractions lyophilised. ¹H-NMR (600 MHz, d₆-DMSO+TFA, 300K) δ: 7.30-7.26 (2H, m), 7.22-7.12 (6H, m), 4.75 (1H, d, J=14.8 Hz), 4.61-4.56 (1H, m), 4.39 (1H, d, J=14.8 Hz), 3.64 (1H, dd, J=12.2, 4.0 Hz, 1H), 3.45 (1H, t, J=12.2 Hz), 3.40-3.36 (1H, m), 3.34 (1H, d, J=13.4 Hz), 3.27 (1H, d, J=13.4 Hz), 2.69 (1H, dd, J=6.3, 13.8 Hz), 2.62 (3H, s), 2.31 (1H, dd, J=13.8, 8.9 Hz). MS (ES) C₂₄H₂₂F₂N₄O₃ requires: 452. found: 453 (M+H)⁺.

### EXAMPLE 12

### (+/−) cis 2-(Dimethylamino)-7-(4-fluorobenzyl)-5-hydroxy-8,8a-dihydro-1H-3,7,8b-triazaacenaphthylene-4,6(2H, 7H)-dione trifluoroacetate salt (M1)

To a solution of cis tert-butyl[7-(4-fluorobenzyl-5-hydroxy-4,6-dioxo-2,4,6,7,8,8a-hexahydro-1H-3,7,8b-triazaacenaphthylen-2-yl]methylcarbamate (J9) in DCM was added TFA and the solution was stirred at room temperature. After 2 hours the reaction mixture was concentrated to dryness under reduced pressure to give cis 7-(4-fluorobenzyl)-5-hydroxy-N-methyl-4,6-dioxo-2,4,6,7,8,8a-hexahydro-1H-3,7,8b-triazaacenaphthylen-2-aminium trifluoroacetate. The residue was dissolved in MeOH, formaldehyde and NaBH₃(CN) (1 equivalent) were added and the suspension was stirred at room temperature. After 30 minutes, the reaction mixture was concentrated under reduced pressure and purified by preparative RP-HPLC, using a gradient of water (0.1% TFA) and acetonitrile (0.1% TFA) as eluants (column: C18). The product was obtained after lyophilization of the pooled product fractions. ¹H-NMR (400 MHz, d₆-DMSO) δ: 10.47 (s, br, 1H), 7.41 (dd, J=8.8, 5.6 Hz, 2H), 4.86 (s, br, 1H), 4.84 (d, J=14.8 Hz, 1H), 4.51 (d, J=14.8 Hz, 1H), 4.39-4.43 (m, 1H), 3.81 (dd, J=3.6, 12.2 Hz, 1H), 3.65 (t, J=12.2 Hz, 1H), 2.73 (s, 6H), 2.73-2.63 (m, 1H), 2.25-2.18 (m, 1H). MS (ES) C₁₈H₁₉FN₄O₃ requires: 358. found: 359 (M+H)⁺.

### EXAMPLE 13

### (+/−) cis N-[7-(4-Fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide (N1)

To a solution of cis 7-(4-fluorobenzyl)-5-hydroxy-N-methyl-4,6-dioxo-2,4,6,7,8,8a-hexahydro-1H-3,7,8b-triazaacenaphthylen-2-aminium trifluoroacetate (prepared as described in Example 12) in DCM were added Et₃N (3 equivalents) and methyl chlorooxoacetate (2 equivalents) and the solution was stirred at room temperature for 30 minutes. The reaction mixture was concentrated under reduced pressure and the residue was dissolved in a solution of Me₂NH in MeOH and the resulting solution stirred at room temperature. After 2 hours the reaction mixture was concentrated under reduced pressure and purified by preparative RP-HPLC, using a gradient of H₂O (0.1% TFA) and MeCN (0.1% TFA) as eluants (column: C18), and the desired product was obtained after lyophilisation. Two patterns of signal corresponding to two conformers 2:1: ¹H-NMR (400 MHz, DMSO) δ: 10.26 (1H, br. s), 7.42-7.38 (2H, m), 7.20 (2H, t, J=8.8 Hz), 5.83 (0.66H, dd, J=10.3, 8.1 Hz), 5.17 (0.33H, dd, J=9.9, 8.1 Hz), 4.86 (1H, dd, J=18.6, 14.8 Hz), 4.53-4.44 (2H, m), 3.79-3.64 (2H, m), 2.99 (2H, s), 2.98 (1H, s), 2.89 (2H, s), 2.88 (1H, s), 2.80 (2H, s), 2.72 (1H, s), 2.61-2.49 (1H, m), 2.14-2.38 (1H, m). MS (ES) C₂₁H₂₂FN₅O₅ requires: 443. found: 444 (M+H)⁺.

### EXAMPLE 14

### (+/−) trans N-[7-(4-Fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide (O1)

To a solution of trans 7-(4-fluorobenzyl)-5-hydroxy-N-methyl-4,6-dioxo-2,4,6,7,8,8a-hexahydro-1H-3,7,8b-triazaacenaphthylen-2-aminium trifluoroacetate (prepared as described in Example 12 but starting from J10) in DCM were added Et₃N (3 equivalents) and methyl chlorooxoacetate (2 equivalents) and the solution was stirred at room temperature for 30 minutes. The reaction mixture was concentrated under reduced pressure and the residue was dissolved in a solution of Me₂NH in MeOH and the resulting solution stirred at room temperature. After 2 hours the reaction mixture was concentrated under reduced pressure and purified by preparative RP-HPLC, using a gradient of H₂O (0.1% TFA) and MeCN (0.1% TFA) as eluants (column: C18), and the desired product was obtained after lyophilisation. Two patterns of signal corresponding to two conformers 2:1: ¹H-NMR (300 MHz, d₆-DMSO) δ: 7.61 (2H, dd, J=10.8, 8.8 Hz), 7.38 (2H, t, J=8.8 Hz), 5.46 (0.66H, d, J=10.1 Hz), 5.21-5.16 (0.33H, m), 5.01-4.80 (3H, m), 3.98-3.93 (2H, m), 3.84 (1H, t, J=11.9 Hz), 3.25-2.96 (9H, m), 2.65-2.44 (1H, m). MS (ES) C₂₁H₂₂FN₅O₅ requires: 443. found: 444 (M+H)⁺.

### EXAMPLE 15

### (+/−) trans N-[7-(3-Chloro-4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide

### Step 1

### (+/−) trans tert-Butyl[7-(3-chloro-4-fluorobenzyl-5-hydroxy-4,6-dioxo-2,4,6,7,8,8a-hexahydro-1H-3,7,8b-triazaacenaphthylen-2-yl]methylcarbamate (P1)

To a suspension of KH (9 equivalents) in THF was added a solution of tert-butyl (5-hydroxy-4,6-dioxo-2,4,6,7,8,8a-hexahydro-1H-3,7,8b-triazaacenaphthylen-2-yl)methylcarbamate (J8) in THF and the reaction mixture was stirred at room temperature for 10 minutes. 3-Chloro-4-fluorobenzyl bromide (2 equivalents) was added and the resulting suspension was stirred at room temperature for 16 hours. AcOH was added and the reaction mixture was concentrated under reduced pressure. The residue was purified by preparative RP-HPLC, using a gradient of H₂O (0.1% TFA) and MeCN (0.1% TFA) as eluants (column: C18) and the product was obtained after lyophilization of the desired fractions. ¹H-NMR (400 MHz, d₆-DMSO) δ: 7.57 (11H, br. s), 7.42-7.39 (2H, m), 5.14-4.92 (1H, m), 4.77-4.67 (1H, m), 4.64 (2H, s), 3.71 (1H, dd, J=11.8, 3.7 Hz), 3.61 (1H, t, J=10.9 Hz), 2.78 (3H, s), 2.38-2.27 (1H, m), 2.22-2.11 (1H, m), 1.39 (6H, s), 1.31 (3H, br. s). MS (ES) C₂₂H₂₄ClFN₄O₅ requires: 478. found: 479 (M+H)⁺.

### Step 2

### (+/−) trans N-[7-(3-Chloro-4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide (P2)

To a solution of (+/−) trans tert-butyl[7-(3-chloro-4-fluorobenzyl-5-hydroxy-4,6-dioxo-2,4,6,7,8,8a-hexahydro-1H-3,7,8b-triazaacenaphthylen-2-yl]methylcarbamate (P1) in DCM was added TFA and the solution was stirred at room temperature for 2 hours. The reaction mixture was concentrated under reduced pressure and the residue was dissolved in DCM, Et₃N (4 equivalents) and methyl chlorooxoacetate (2 equivalents) were added and the solution was stirred at room temperature for 2 hours. The solvents were removed under reduced pressure and the residue was dissolved in a solution of Me₂NH in MeOH. The resulting solution was stirred at room temperature for 16 hours and was then concentrated under reduced pressure and purified by preparative RP-HPLC (gradient of H₂O (0.1% TFA) and MeCN (0.1% TFA) as eluants, column: C18). The desired product was obtained after lyophilisation of the desired fractions. Two patterns of signal corresponding to two conformers 2:1: ¹H-NMR (300 MHz, d₆-DMSO) δ: 7.78 (1H, d, J=6.4 Hz), 7.59 (2H, app. d, J=7 Hz), 5.50 (0.66H, d, J=9.0 Hz), 5.17 (0.33H, dd, J=6.6, 4.4 Hz), 5.07-4.91 (1H, m), 4.88 (1H, s), 4.82-4.73 (1H, m), 4.02-3.79 (2H, m), 3.24-2.95 (9H, m), 2.70-2.40 (2H, m). MS (ES) C₂₁H₂₁ClFN₅O₅ requires: 477. found: 478 (M+H)⁺.

### EXAMPLE 16

### 2-(3-Chloro-4-fluorobenzyl)-9-hydroxy-N,N-dimethyl-1,8-dioxo-1,3,4,8-tetrahydro-2H-pyrido[1,2-α]pyrazine-6-carboxamide (Q8) and 2-(3-chloro-4-fluorobenzyl)-9-hydroxy-1,8-dioxo-1,3,4,8-tetrahydro-2H-pyrido[1,2-α]pyrazine-6-carboxylic acid (Q9)

### Step 1

### {3-(Benzyloxy)-4-[(4-methoxybenzyl)oxy]pyridin-2-yl}methyl acetate (Q1)

mCPBA (2.0 equivalents) was added portionwise over 15 minutes to a stirred solution of the 3-(benzyloxy)-4-[(4-methoxybenzyl)oxy]-2-methylpyridine (A1) (1.0 equivalent) in DCM at 0° C. The reaction was stirred overnight, gradually warming to room temperature. The reaction mixture was then diluted with DCM and washed with 1 M NaOH solution (3 times), then brine and dried (Na₂SO₄). The desired pyridine-N-oxide was used without further purification. MS (ES) C₂₁H₂₁NO₄ requires: 351, found: 352 (M+H⁺). The residue (1 equivalent) was taken up in excess Ac₂O, and the resulting mixture was heated at 130° C. for 90 minutes. After cooling to room temperature, the mixture was concentrated under reduced pressure and was then taken up in DCM. The solution was washed with saturated NaHCO₃ solution and brine, and then dried (Na₂SO₄) prior to concentrating under reduced pressure. MS (ES) C₂₃H₂₃NO₅ requires: 393. found: 394 (M+H⁺).

### Step 2

### {3-(Benzyloxy)-6-cyano-4-[(4-methoxybenzyl)oxy]pyridin-2-yl}methyl acetate (Q2)

mCPBA (1.3 equivalent) was added in one portion to a stirred pyridine (Q1) (1.0 equivalent) in chloroform. The reaction mixture was stirred at 45° C. for 45 minutes and at 60° C. for 60 minutes. After cooling to room temperature, mixture was diluted with chloroform and washed with saturated NaHCO₃ solution, dried (Na₂SO₄), filtered and concentrated under reduced pressure to yield after trituration with ethyl ether the desired N-oxide. MS (ES) C₂₃H₂₃NO₆ requires: 409. found: 410 (M+H⁺). ¹H NMR (300 MHz, CDCl₃) δ 8.15 (1H, d, J=5.6 Hz), 7.40-7.30 (7H, m), 6.96 (2H, d, J=7.8 Hz), 6.82 (1H, d, J=5.6 Hz), 5.35 (2H, s), 5.12 (4H, s), 3.81 (3H, s), 2.05 (3H, s). The N-oxide was taken up in dry DCM and treated with TMSCN (2 equivalents). After 5 minutes stirring at room temperature diethylcarbamyl chloride (2 equivalents) was added and stirring prolonged for 24 hours. The mixture was poured in DCM and washed with 1 N NaOH (3 times) and brine, dried (Na₂SO₄) and concentrated under reduced pressure. Product was obtained from the residue upon trituration with Et₂O. ¹H NMR (300 MHz, CDCl₃) δ 7.42-7.25 (8H, m), 6.95 (2H, d, J=7.8 Hz), 5.18 (4H, s), 5.15 (2H, s), 3.88 (3H, s), 2.11 (3H, s). MS (ES) C₂₄H₂₂N₂O₅ requires: 418. found: 419 (M+H⁺).

### Step 3

### Methyl 5-(benzyloxy)-6-(hydroxymethyl)-4-[(4-methoxybenzyl)oxy]pyridine-2-carboxylate (Q3)

K₂CO₃ (1 equivalent) was added in one portion to a suspension of the nitrile (Q2) in MeOH and the mixture was stirred at room temperature for 90 minutes. The mixture was cooled to 0° C. and 1N HCl (2.5 equivalents) was added dropwise. The reaction was stirred overnight gradually warming to room temperature. The volatiles were partially evaporated under reduced pressure and residue was taken in EtOAc and washed with sat. aq. NaHCO₃ solution (3 times). The organics were dried (Na₂SO₄) and concentrated under reduced pressure and the crude product was used without further purification. MS (ES) C₂₃H₂₃NO₆ requires: 409. found: 410 (M+H⁺).

### Step 4

### Methyl 5-(benzyloxy)-6-formyl-4-[(4-methoxybenzyl)oxy]pyridine-2-carboxylate (Q4)

MnO₂ (25 equivalents) was added to a stirred solution of the alcohol (Q3) in CHCl₃ and the mixture was refluxed for 60 minutes. The reaction was cooled to room temperature and filtered under vacuum. The solid cake was extensively washed with CHCl₃ and filtrate was evaporated to an oily residue under reduced pressure. This residue was purified by flash chromatography on silica eluting with 33% EtOAc/Petroleum Ether to give the desired aldehyde. ¹H NMR (400 MHz, CDCl₃) δ 10.21 (1H, s), 7.99 (1H, s), 7.40-7.22 (7H, m), 6.95 (2H, d, J=7.8 Hz), 5.28 (2H, s), 5.20 (2H, s), 4.03 (3H, s), 3.83 (3H, s). MS (ES) C₂₃H₂₁NO₆ requires: 407. found: 408 (M+H⁺).

### Step 5

### 3-(Benzyloxy)-4-[(4-methoxybenzyl)oxy]-6-(methoxycarbonyl)pyridine-2-carboxylic acid (Q5)

Sulfamic acid (1.4 equivalents) and sodium chlorite (1.1 equivalents) were added sequentially to a stirred solution of the aldehyde (Q4) (1.0 equivalents) in acetone and water. The resulting mixture was stirred at room temperature for 90 minutes and then the acetone was removed under reduced pressure. The organics were extracted with DCM, and then the DCM extracts were washed with brine. The extracts were dried (Na₂SO₄) and concentrated under reduced pressure to yield the desired acid. MS (ES) C₂₃H₂₁NO₇ requires: 423. found: 424 (M+H⁺).

### Step 6

### Methyl 5-(benzyloxy)-6-{[(3-chloro-4-fluorobenzyl)(2-hydroxyethyl)amino]carbonyl}-4-hydroxypyridine-2-carboxylate (Q6)

PyBOP (1.2 equivalents) was added to a stirred solution of the acid (Q5) (1.0 equivalent), (2-{[tert-butyl(dimethyl)silyl]oxy}ethyl)(3-chloro-4-fluorobenzyl)amine (1.2 equivalents) [Prepared from 3-chloro-4-fluorobenzylamine and 2-{[tert-butyl(dimethyl)silyl]-oxy}ethanal with NaBH₄ in MeOH] and Et₃N (1.5 equivalents) in DCM and the mixture was stirred at room temperature overnight. The reaction was diluted with DCM and washed with sat. aq. NaHCO₃ solution and brine and then dried (Na₂SO₄). The resulting solution was concentrated under reduced pressure and purified by column chromatography on silica eluting with 30% EtOAc/petroleum ether. The isolated amide was taken up in DCM/TFA (9/1) and the resulting solution was stirred 90 minutes at room temperature. The volatiles were removed under reduced pressure and the crude residue was triturated with Et₂O to yield the desired alcohol. MS(ES) C₂₄H₂₂ClFN₂O₆ requires: 488. found: 489 (M+H⁺).

### Step 7

### Methyl 9-(benzyloxy)-2-(3-chloro-4-fluorobenzyl)-1,8-dioxo-1,3,4,8-tetrahydro-2H-pyrido[1,2-α]pyrazine-6-carboxylate (Q7)

DEAD (1.5 equivalent) was added dropwise over 10 minutes to a stirred suspension of the alcohol (Q6) (1.0 equivalent) and PPh₃ (1.5 equivalents) in DCM at room temperature. The mixture became homogeneous and was stirred for 60 minutes, then the solvent was removed under reduced pressure and desired bicycle 8 was purified by column chromatography on silica eluting with CHCl₃/EtOAc/MeOH (8:2:0.1). ¹H NMR (400 MHz, CDCl₃ δ 7.60 (2H, d, J=6.9 Hz), 7.44-7.05 (7H, m), 5.39 (2H, s), 4.61 (2H, s), 4.19-4.11 (2H, m), 3.92 (3H, s), 3.41-3.38 (2H, m). MS(ES) C₂₄H₂₀ClFN₂O₅ requires: 470. found: 471 (M+H⁺).

### Step 8

### 2-(3-Chloro-4-fluorobenzyl)-9-hydroxy-N,N-dimethyl-1,8-dioxo-1,3,4,8-tetrahydro-2H-pyrido[1,2-α]pyrazine-6-carboxamide (Q8) and 2-(3-chloro-4-fluorobenzyl)-9-hydroxy-1,8-dioxo-1,3,4,8-tetrahydro-2H-pyrido[1,2-α]pyrazine-6-carboxylic acid (Q9)

The above methyl ester (Q7) was taken up in 2M solution of diethylamine in MeOH and mixture was heated in a sealed tube at 80° C. for 120 minutes. The reaction mixture was cooled to room temperature, evaporated under reduced pressure. The crude amide was taken up in THF, 6N HCl (excess) was added and mixture stirred at 60° C. overnight. The volatiles were removed under reduced pressure and residue was purified by reverse phase HPLC to yield two products after lyophilisation of the desired fractions.

2-(3-Chloro-4-fluorobenzyl)-9-hydroxy-N,N-dimethyl-1,8-dioxo-1,3,4,8-tetrahydro-2H-pyrido[1,2-α]pyrazine-6-carboxamide (Q8): ¹H NMR (400 MHz, CD₃CN δ 7.58 (1H, d, J=5.9 Hz), 7.39-7.21 (2H, m), 7.19 (1H, s), 4.75-4.71 (2H, m), 4.22-4.10 (2H, m), 3.73-3.68 (2H, m), 3.05 (3H, s), 2.91 (3H, s). MS(ES) C₁₈H₁₇ClFN₃O₄ requires: 393. found: 394 (M+H⁺).

2-(3-Chloro-4-fluorobenzyl)-9-hydroxy-1,8-dioxo-1,3,4,8-tetrahydro-2H-pyrido[1,2-α]pyrazine-6-carboxylic acid (Q9): ¹H NMR (400 MHz, d₆-DMSO δ 7.63 (1H, d, J=5.9 Hz), 7.48-7.40 (2H, m), 6.61 (1H, s), 4.73 (2H, s), 4.48-4.42 (2H, m), 3.70-3.66 (2H, m). MS (ES) C₁₆H₁₂ClFN₂O₅ requires: 366. found: 367 (M+H⁺).

### EXAMPLE 17

### 4-(Carboxymethyl)-2-(4-fluorobenzyl)-9-hydroxy-1,8-dioxo-1,3,4,8-tetrahydro-2H-pyrido[1,2-α]pyrazin-5-ium trifluoroacetate (R4) and 4-(2-Ethoxy-2-oxoethyl)-2-(4-fluorobenzyl)-9-hydroxy-1,8-dioxo-1,3,4,8-tetrahydro-2H-pyrido[1,2-α]pyrazin-5-ium trifluoroacetate (R5)

### Step 1

### Ethyl 4-[(4-fluorobenzyl)amino]but-2-enoate (R1)

A suspension of 50% KF/Celite in MeCN was treated with 4-fluorobenzylamine (1 equivalent) and Et₃N (2 equivalents) and the mixture was cooled to 0° C. Ethyl 4-bromocrotonate (1 equivalent) was added dropwise over 10 minutes and the mixture was warmed to room temperature and stirred for 2 hours. The mixture was filtered under vacuum and the solvent removed under reduced pressure to yield the desired amine. ¹H NMR (300 MHz, CDCl₃) δ 7.38-7.20 (3H, m), 7.05-6.93 (2H, m), 6.02 (1H, d, J=14.0 Hz), 4.12 (2H, q, J=7.2 Hz), 3.78 (2H, s), 3.42 (2H, d, J=5.5 Hz), 1.29 (3H, t, J=7.2 Hz). MS (ES) C₁₃H₁₆FNO₂ requires: 237. found: 238 (M+H⁺).

### Step 2

### Ethyl (2E)-4-[({3-(benzyloxy)-4-[(4-methoxybenzyl)oxy]pyridin-2-yl}carbonyl)(4-fluorobenzyl)amino]but-2-enoate (R2)

PyBOP (1.2 equivalents) was added to a stirred solution of 3-(benzyloxy)-4-[(4-methoxybenzyl)oxy]pyridine-2-carboxylic acid (A4) (1.0 equivalent), ethyl 4-[(4-fluorobenzyl)amino]but-2-enoate (R1) (1.2 equivalents), and Et₃N (1.3 equivalents) in DMF and the mixture was stirred at room temperature overnight. Xylene was added and the reaction mixture was concentrated under reduced pressure. The residue was taken up in EtOAc and was washed with sat. aq. NaHCO₃ solution and brine and then dried (Na₂SO₄). The resulting solution was concentrated under reduced pressure and purified by column chromatography on silica eluting with 60-100% EtOAc/petroleum ether to yield the desired amide. MS(ES) C₃₄H₃₃FN₂O₆ requires: 584. found: 585 (M+H⁺).

### Step 3

### Ethyl[9-(benzyloxy)-2-(4-fluorobenzyl)-1,8-dioxo-1,3,4,8-tetrahydro-2H-pyrido[1,2-α]pyrazin-4-yl]acetate (R3)

The amide (R2) (1 equivalent) was taken up in THF and 3N HCl (15 equivalents) and the mixture was stirred at room temperature overnight. The reaction mixture was neutralized with 2 N NaOH solution and the organics were extracted with DCM (3 times). The organic extracts were concentrated under reduced pressure and used without further purification in the next step. MS (ES) C₂₆H₂₅N₂O₅F requires: 464. found: 465 (M+H⁺).

### Step 4

### 4-(Carboxymethyl)-2-(4-fluorobenzyl)-9-hydroxy-1,8-dioxo-1,3,4,8-tetrahydro-2H-pyrido[1,2-α]pyrazin-5-ium trifluoroacetate (R4) and 4-(2-Ethoxy-2-oxoethyl)-2-(4-fluorobenzyl)-9-hydroxy-1,8-dioxo-1,3,4,8-tetrahydro-2H-pyrido[1,2-α]pyrazin-5-ium trifluoroacetate (R5)

The ester (R3) (1 equivalent) was taken up in MeOH and 1 M HCl (1 equivalent) was added followed up 10% Pd/C. The reaction was stirred under an H₂ atmosphere for 1 hour and then the H₂ was evacuated and the reaction was filtered. The filter cake washed with MeOH and the filtrate concentrated under reduced pressure. The residue was purified by preparative RP-HPLC (using H₂ (0.1% TFA) and MeCN (0.1% TFA) as eluants, column: C18) and the desired fractions lyophilized to yield first the acid (R4) and then the ester (R5). The desired fractions were lyophilized.

4-(Carboxymethyl)-2-(4-fluorobenzyl)-9-hydroxy-1,8-dioxo-1,3,4,8-tetrahydro-2H-pyrido[1,2-α]pyrazin-5-ium trifluoroacetate (R4): ¹H NMR (400 MHz, d₆-DMSO) δ 7.69 (1H, d, J=5.5 Hz), 7.50-7.38 (2H, m), 7.21 (2H, t, J=6.7 Hz), 6.28 (1H, d, J=5.5 Hz), 4.85 (1H, d, J=13.0 Hz), 4.72 (1H, br. s), 4.52 (1H, d, J=13.0 Hz), 3.97 (1H, d, J=8.8 Hz), 3.54 (1H, d, J=8.8 Hz), 2.80-2.30 (2H, m). MS (ES) C₁₇H₁₅FN₂O₅ requires: 346. found: 347 (M+H⁺).

4-(2-Ethoxy-2-oxoethyl)-2-(4-fluorobenzyl)-9-hydroxy-1,8-dioxo-1,3,4,8-tetrahydro-2H-pyrido[1,2-α]pyrazin-5-ium trifluoroacetate (R5): ¹H NMR (300 MHz, d₆-DMSO) δ 7.82 (1H, d, J=6.5 Hz), 7.47-7.38 (2H, m), 7.20 (2H, t, J=8.8 Hz), 6.48 (1H, d, J=6.5 Hz), 4.92 (1H, d, J=12.0 Hz), 4.90-4.82 (1H, m), 4.46 (1H, d, J=12.0 Hz), 4.10-3.85 (3H, m), 3.60-3.30 (2H, m), 2.80-2.30 (2H, m), 1.12 (3H, t, J=7.0 Hz). MS (ES) C₁₉H₁₉FN₂O₅ requires: 374. found: 377 (M+H⁺).

### EXAMPLE 18

### 4-[2-(Dimethylamino)-2-oxoethyl]-2-(4-fluorobenzyl)-9-hydroxy-1,8-dioxo-1,3,4,8-tetrahydro-2H-pyrido[1,2-α]pyrazin-5-ium trifluoroacetate

### Step 1

### [9-(Benzyloxy)-2-(4-fluorobenzyl)-1,8-dioxo-1,3,4,8-tetrahydro-2H-pyrido[1,2-α]pyrazin-4-yl]acetic acid (S1)

Ethyl[9-(benzyloxy)-2-(4-fluorobenzyl)-1,8-dioxo-1,3,4,8-tetrahydro-2H-pyrido[1,2-α]pyrazin-4-yl]acetate (R3) (1.0 equivalent) was taken up in MeOH, and KOH (5 equivalents) and H₂O were added. The reaction mixture was heated at 50° C. for 30 minutes and then was quenched by the addition of 1M HCl to neutralize the base. The MeOH was removed under reduced pressure and the organics were extracted with DCM (2 times). Then the DCM extracts were dried (Na₂SO₄) and concentrated under reduced pressure to yield the desired acid which was used without further purification. MS (ES) C₂₄H₂₁FN₂O₅ requires: 436. found: 437 (M+H⁺).

### Step 2

### 4-[2-(Dimethylamino)-2-oxoethyl]-2-(4-fluorobenzyl)-9-hydroxy-1,8-dioxo-1,3,4,8-tetrahydro-2H-pyrido[1,2-α]pyrazin-5-ium trifluoroacetate (S2)

PyBOP (1.2 equivalents) was added to a stirred mixture of the acid (S1) (1.0 equivalent), a solution of Me₂NH in THF (5 equivalents), and Et₃N (1.2 equivalents) in DCM. The resulting mixture was stirred at room temperature for 3 hours. The reaction mixture was concentrated under reduced pressure whilst azeotroping with xylene and the crude residue was purified by preparative RP-HPLC (using H₂O (0.1% TFA) and MeCN (0.1% TFA) as eluants, column: C18) to yield 9-(benzyloxy)-4-[2-(dimethylamino)-2-oxoethyl]-2-(4-fluorobenzyl)-1,8-dioxo-1,3,4,8-tetrahydro-2H-pyrido[1,2-α]pyrazin-5-ium trifluoroacetate. MS (ES) C₂₆H₂₆FN₃O₄ requires: 463. found: 464 (M+H⁺). The amide (1 equivalent) was taken up in MeOH and 10% Pd/C was added. The reaction was stirred under an H₂ atmosphere for 90 minutes and then the H₂ was evacuated and the reaction was filtered. The filter cake washed with MeOH and the filtrate concentrated under reduced pressure. The residue was purified by preparative RP-HPLC (using H₂O (0.1% TFA) and MeCN (0.1% TFA) as eluants, column: C18) and the desired fractions were lyophilized to yield the desired amide. ¹H NMR (300 MHz, d₆-DMSO) δ 7.95 (1H, d, J=6.0 Hz), 7.50-7.38 (2H, m), 7.21 (2H, t, J=8.0 Hz), 6.60 (1H, d, J=6.0 Hz), 5.12 (1H, d, J=12.5 Hz), 4.90-4.78 (1H, m), 4.28 (1H, d, J=12.5 Hz), 4.03 (1H, dd, J=8.5, 1.5 Hz), 3.57 (1H, d, J=8.5 Hz), 2.68 (3H, s), 2.63 (3H, s), 2.60-2.40 (2H, m). MS (ES) C₁₉H₂₀FN₃O₄ requires: 373. found: 374 (M+H⁺).

### EXAMPLE 19

### 2-(4-Fluorobenzyl)-9-hydroxy-1,8-dioxo-4-(2-pyrrolidinium-1-ylethyl)-1,3,4,8-tetrahydro-2H-pyrido[1,2-α]pyrazin-5-ium bis(trifluoroacetate)

### Step 1

### 9-(Benzyloxy)-2-(4-fluorobenzyl)-4-(2-hydroxyethyl)-3,4-dihydro-2H-pyrido[1,2-α]pyrazine-1,8-dione (T1)

LiAlH₄ (3.0 equivalents) was added in one portion to a stirred solution of ethyl[9-(benzyloxy)-2-(4-fluorobenzyl)-1,8-dioxo-1,3,4,8-tetrahydro-2H-pyrido[1,2-α]pyrazin-4-yl]acetate (R3) (1.0 equivalent) in THF at room temperature. The mixture was stirred for 3 hours and further portions of LiAlH₄ (2.0 equivalents) were added until complete reaction was observed. The reaction was quenched by careful addition of a sat. aq. solution of Rochelle's salt and the resulting mixture was stirred vigorously for 30 minutes. This mixture was extracted with DCM (5 times). These DCM extracts were concentrated a little under reduced pressure, washed with brine, dried (Na₂SO₄) and concentrated under reduced pressure to yield the desired alcohol which was used without further purification. MS (ES) C₂₄H₂₃FN₂O₄ requires: 422. found: 423 (M+H⁺).

### Step 2

### [9-(Benzyloxy)-2-(4-fluorobenzyl)-1,8-dioxo-1,3,4,8-tetrahydro-2H-pyrido[1,2-α]pyrazin-4-yl]acetaldehyde (T2)

The alcohol (T1) was oxidized under standard Swern conditions as described in Example 1 step 3 to yield the desired aldehyde. MS(ES) C₂₄H₂₁FN₂O₄ requires: 420. found: 421 (M+H⁺).

### Step 3

### 2-(4-Fluorobenzyl)-9-hydroxy-1,8-dioxo-4-(2-pyrrolidinium-1-ylethyl)-1,3,4,8-tetrahydro-2H-pyrido[1,2-α]pyrazin-5-ium bis(trifluoroacetate)

The aldehyde (Q2) was taken up in MeOH and treated with pyrrolidine (10 equivalents), AcOH (10 equivalents) and finally NaBH₃(CN) (6 equivalents). The mixture was stirred at room temperature for 12 hours and was then concentrated under reduced pressure. The residue was treated with 0.5 N NaOH solution and was then extracted with DCM (3 times). The DCM extracts were dried (Na₂SO₄) and the concentrated under reduced pressure to yield 9-(benzyloxy)-2-(4-fluorobenzyl)-4-(2-pyrrolidin-1-ylethyl)-3,4-dihydro-2H-pyrido[1,2-α]pyrazine-1,8-dione. MS(ES) C₂₈H₃₀FN₃O₃ requires: 475. found: 476 (M+H⁺). The amine was taken up in MeOH and 1M HCl (1 equivalent) was added, followed up 10% Pd/C. The reaction was stirred under an H₂ atmosphere for 3 hour and then the H₂ was evacuated and the reaction was filtered. The filter cake washed with MeOH and the filtrate concentrated under reduced pressure. The residue was purified by preparative RP-HPLC (using H₂O (0.1% TFA) and MeCN (0.1% TFA) as eluants, column: C18) and the desired fractions were lyophilized to yield the desired amine.TFA salt.

¹H NMR (400 MHz, d₆-DMSO) δ 9.71 (1H, br. s), 7.63 (1H, d, J=5.2 Hz), 7.51-7.39 (2H, m), 7.22 (2H, t, J=8.0 Hz), 6.24 (1H, d, J=5.2 Hz), 4.79 (1H, d, J=10.5 Hz), 4.68 (1H, d, J=10.5 Hz). 4.41 (1H, br. s), 4.05-2.80 (8H, m), 2.05-1.70 (6H, m). MS (ES) C₂₁H₂₄FN₃O₃ requires: 385. found: 386 (M+H⁺).

Table 1 below lists compounds of the present invention. The table provides the structure and name of each compound, the mass of its molecular ion plus 1 (M⁺) or molecular ion minus 1 (M⁻) as determined via ES, and a reference to the preparative example that is, or is representative of, the procedure employed to prepare the compound.

| Structure | Name | Ex. | M + H⁺ |
|---|---|---|---|
| 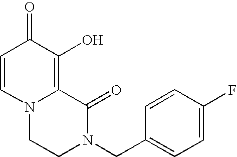 | 2-(4-Fluorobenzyl)-9-hydroxy-3,4-dihydro-2H-pyrido[1,2-a]pyrazine-1,8-dione | 1 | 289 |
| 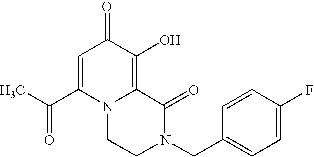 | 6-Acetyl-2-(4-fluorobenzyl)-9-hydroxy-3,4-dihydro-2H-pyrido[1,2-a]pyrazine-1,8-dione | 1 | 331 |
| 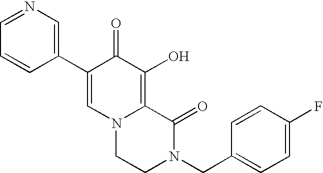 | 2-(4-Fluorobenzyl)-9-hydroxy-7-pyridin-3-yl-3,4-dihydro-2H-pyrido[1,2-a]pyrazine-1,8-dione | 2 | 366 |
| 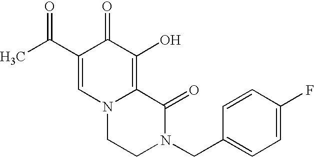 | 7-Acetyl-2-(4-fluorobenzyl)-9-hydroxy-3,4-dihydro-2H-pyrido[1,2-a]pyrazine-1,8-dione | 3 | 331 |
| 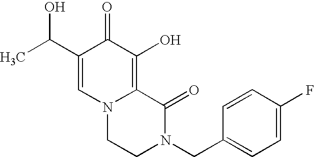 | 2-(4-Fluorobenzyl)-9-hydroxy-7-(1-hydroxyethyl)-3,4-dihydro-2H-pyrido[1,2-a]pyrazine-1,8-dione | 4 | 333 |
| 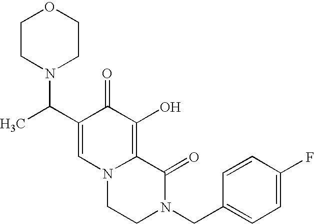 | 2-(4-Fluorobenzyl)-9-hydroxy-7-(1-morpholin-4-ylethyl)-3,4-dihydro-2H-pyrido[1,2-a]pyrazine-1,8-dione | 5 | 402 |
| 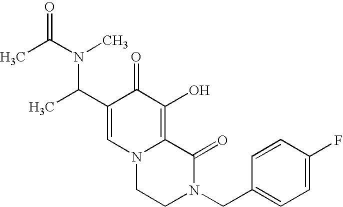 | N-{1-[2-(4-Fluorobenzyl)-9-hydroxy-1,8-dioxo-1,3,4,8-tetrahydro-2H-pyrido[1,2-a]pyrazin-7-yl]ethyl}-N-methylacetamide | 6 | 388 |
| 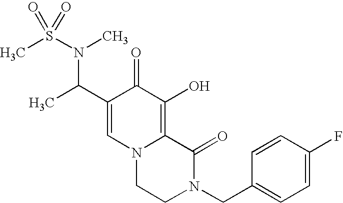 | N-{1-[2-(4-Fluorobenzyl)-9-hydroxy-1,8-dioxo-1,3,4,8-tetrahydro-2H-pyrido[1,2-a]pyrazin-7-yl]ethyl}-N-methylmethane-sulfonamide | 6 | 424 |
| 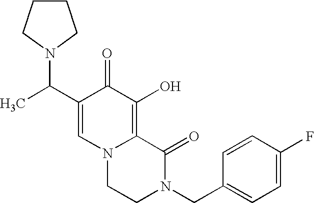 | 2-(4-Fluorobenzyl)-9-hydroxy-7-(1-pyrrolidin-1-ylethyl)-3,4-dihydro-2H-pyrido[1,2-a]pyrazine-1,8-dione | 5 | 386 |
| 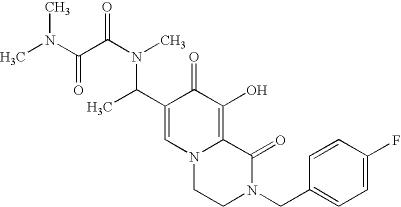 | N-{1-[2-(4-Fluorobenzyl)-9-hydroxy-1,8-dioxo-1,3,4,8-tetra-hydro-2H-pyrido[1,2-a]pyrazin-7-yl]ethyl}-N,N′,N′-trimethylethane-diamide | 6 | 445 |
| 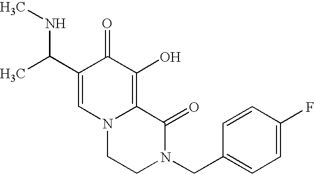 | 2-(4-Fluorobenzyl)-9-hydroxy-7-[1-(methylamino)ethyl]-3,4-dihydro-2H-pyrido[1,2-a]pyrazine-1,8-dione | 5 | 346 |
| 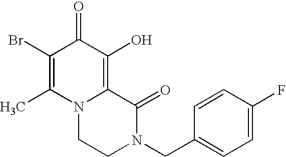 | 7-Bromo-2-(4-fluorobenzyl)-9-hydroxy-6-methyl-3,4-dihydro-2H-pyrido[1,2-a]pyrazine-1,8-dione | 9 | 381 |
| 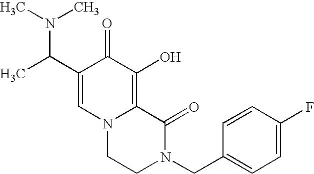 | 7-[1-(Dimethylamino)ethyl]-2-(4-fluorobenzyl)-9-hydroxy-3,4-dihydro-2H-pyrido[1,2-a]pyrazine-1,8-dione | 5 | 360 |
| 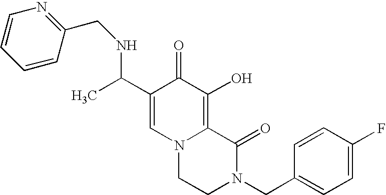 | 2-(4-Fluorobenzyl)-9-hydroxy-7-{1-[(pyridin-2-ylmethyl)amino]ethyl}-3,4-dihydro-2H-pyrido[1,2-a]pyrazine-1,8-dione | 5 | 423 |
| 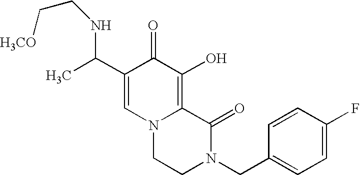 | 2-(4-Fluorobenzyl)-9-hydroxy-7-{1-[(2-methoxyethyl)amino]ethyl}-3,4-dihydro-2H-pyrido[1,2-a]pyrazine-1,8-dione | 5 | 390 |
| 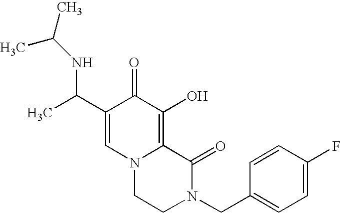 | 2-(4-Fluorobenzyl)-9-hydroxy-7-[1-(isopropylamino)ethyl]-3,4-dihydro-2H-pyrido[1,2-a]pyrazine-1,8-dione | 5 | 374 |
| 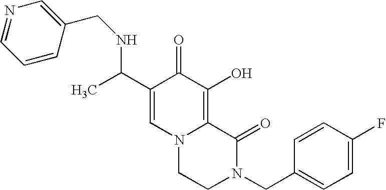 | 2-(4-Fluorobenzyl)-9-hydroxy-7-{1-[(pyridin-3-ylmethyl)amino]ethyl}-3,4-dihydro-2H-pyrido[1,2-a]pyrazine-1,8-dione | 5 | 423 |
| 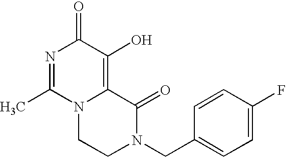 | 2-(4-Fluorobenzyl)-9-hydroxy-6-methyl-3,4-dihydro-2H-pyrazino[1,2-c]pyrimidine-1,8-dione | 7 | 304 |
| 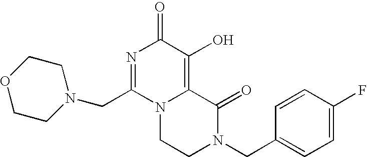 | 2-(4-Fluorobenzyl)-9-hydroxy-6-(morpholin-4-ylmethyl)-3,4-dihydro-2H-pyrazino[1,2-c]pyrimidine-1,8-dione | 8 | 389 |
| 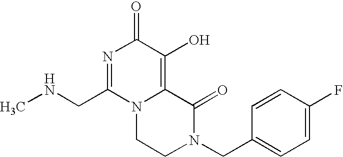 | 2-(4-Fluorobenzyl)-9-hydroxy-6-[(methylamino)methyl]-3,4-dihydro-2H-pyrazino[1,2-c]pyrimidine-1,8-dione | 8 | 333 |
| 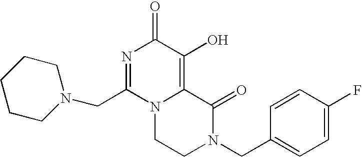 | 2-(4-Fluorobenzyl)-9-hydroxy-6-(piperidin-1-ylmethyl)-3,4-dihydro-2H-pyrazino[1,2-c]pyrimidine-1,8-dione | 8 | 387 |
| 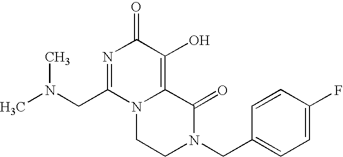 | 6-[(Dimethylamino)methyl]-2-(4-fluorobenzyl)-9-hydroxy-3,4-dihydro-2H-pyrazino[1,2-c]pyrimidine-1,8-dione | 8 | 347 |
| 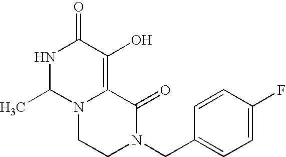 | 2-(4-Fluorobenzyl)-9-hydroxy-6-methyl-3,4,6,7-tetrahydro-2H-pyrazino[1,2-c]pyrimidine-1,8-dione | 7 | 306 |
| 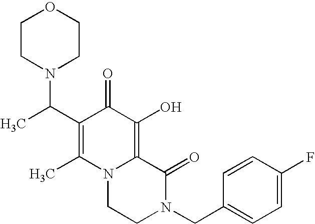 | 2-(4-fluorobenzyl)-9-hydroxy-6-methyl-7-(1-morpholin-4-ylethyl)-3,4-dihydro-2H-pyrido[1,2-a]pyrazine-1,8-dione | 5 | 416 |
| 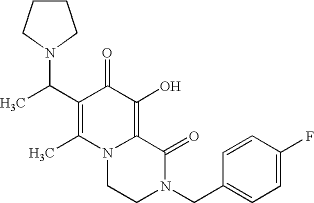 | 2-(4-fluorobenzyl)-9-hydroxy-6-methyl-7-(1-pyrrolidin-1-ylethyl)-3,4-dihydro-2H-pyrido[1,2-a]pyrazine-1,8-dione | 5 | 400 |
| 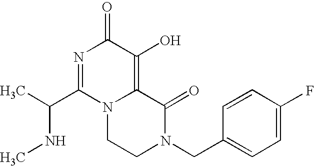 | 2-(4-fluorobenzyl)-9-hydroxy-6-[1-(methylamino)ethyl]-3,4-dihydro-2H-pyrazino[1,2-c]pyrimidine-1,8-dione | 8 | 347 |
| 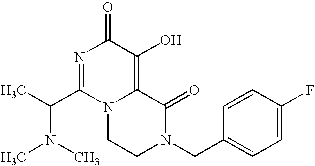 | 6-[1-(dimethylamino)ethyl]-2-(4-fluorobenzyl)-9-hydroxy-3,4-dihydro-2H-pyrazino[1,2-c]pyrimidine-1,8-dione | 8 | 361 |
| 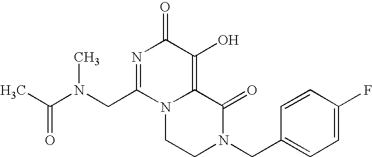 | N-{[2-(4-fluorobenzyl)-9-hydroxy-1,8-dioxo-1,3,4,8-tetrahydro-2H-pyrazino[1,2-c]pyrimidin-6-yl]methyl}-N-methylacetamide | 8 | 375 |
| 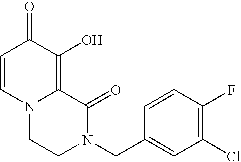 | 2-(3-chloro-4-fluorobenzyl)-9-hydroxy-3,4-dihydro-2H-pyrido[1,2-α]pyrazine-1,8-dione, TFA salt | 1 | 323 |
| 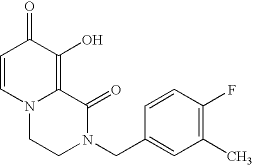 | 2-(4-fluoro-3-methylbenzyl)-9-hydroxy-3,4-dihydro-2H-pyrido[1,2-α]pyrazine-1,8-dione, TFA salt | 1 | 303 |
| 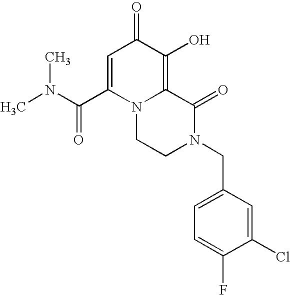 | 2-(3-chloro-4-fluorobenzyl)-9-hydroxy-N,N-dimethyl-1,8-dioxo-1,3,4,8-tetrahydro-2H-pyrido[1,2-α]pyrazine-6-carboxamide, TFA salt | 16 | 394 |
| 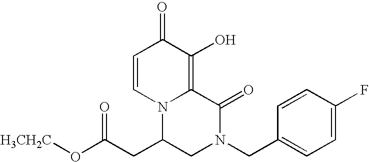 | ethyl [2-(4-fluorobenzyl)-9-hydroxy-1,8-dioxo-1,3,4,8-tetrahydro-2H-pyrido[1,2-α]pyrazin-4-yl]acetate, TFA salt | 17 | 375 |
| 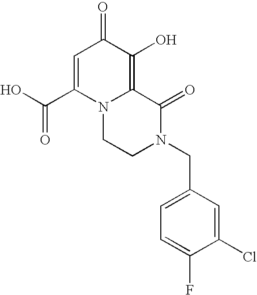 | 2-(3-chloro-4-fluorobenzyl)-9-hydroxy-1,8-dioxo-1,3,4,8-tetrahydro-2H-pyrido[1,2-α]pyrazine-6-carboxylic acid | 16 | 366 |
| 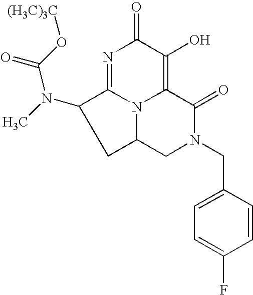 | cis tert-butyl [7-(4-fluorobenzyl)-5-hydroxy-4,6-dioxo-2,4,6,7,8,8a-hexahydro-1H-3,7,8b-triazaacenaphthylen-2-yl]methylcarbamate | 10 | 445 |
| 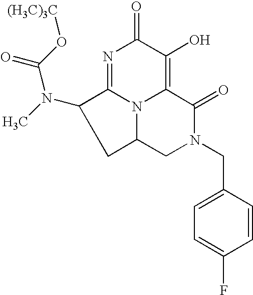 | trans tert-butyl [7-(4-fluorobenzyl)-5-hydroxy-4,6-dioxo-2,4,6,7,8,8a-hexahydro-1H-3,7,8b-triazaacenaphthylen-2-yl]methylcarbamate | 10 | 445 |
| 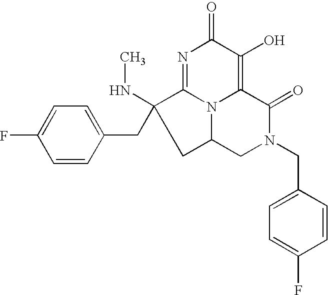 | 2,7-bis(4-fluorobenzyl)-5-hydroxy-2-(methylamino)-8,8a-dihydro-1H-3,7,8b-triazaacenaphthylene-4,6(2H,7H)-dione, TFA salt | 11 | 453 |
| 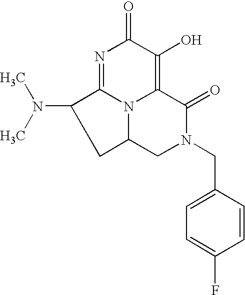 | cis 2-(dimethylamino)-7-(4-fluorobenzyl)-5-hydroxy-8,8a-dihydro-1H-3,7,8b-triazaacenaphthylene-4,6(2H,7H)-dione, TFA salt | 12 | 359 |
| 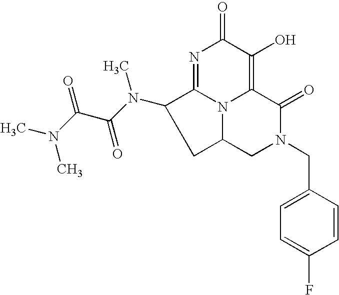 | cis N-[7-(4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide | 13 | 444 |
| 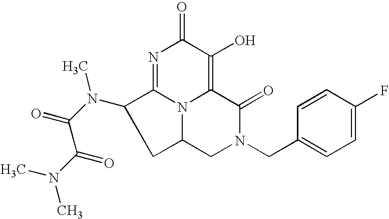 | trans N-[7-(4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide | 14 | 444 |
| 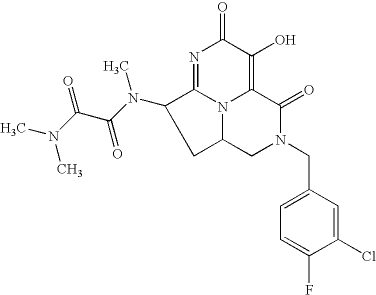 | N-[7-(3-chloro-4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide | 15 | 478 |
| 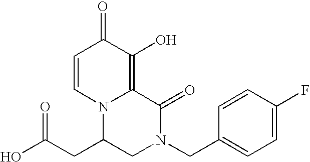 | [2-(4-fluorobenzyl)-9-hydroxy-1,8-dioxo-1,3,4,8-tetrahydro-2H-pyrido[1,2-α]pyrazin-4-yl]aceticacid, TFA salt | 17 | 347 |
| 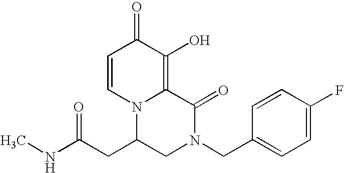 | 2-[2-(4-fluorobenzyl)-9-hydroxy-1,8-dioxo-1,3,4,8-tetrahydro-2H-pyrido[1,2-α]pyrazin-4-yl]-N-methylacetamide, TFA salt | 18 | 360 |
| 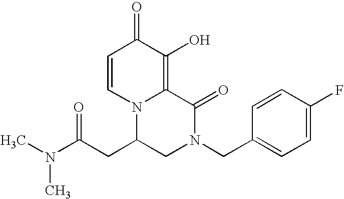 | 2-[2-(4-fluorobenzyl)-9-hydroxy-1,8-dioxo-1,3,4,8-tetrahydro-2H-pyrido[1,2-α]pyrazin-4-yl]-N,N-dimethylacetamide, TFA salt | 18 | 374 |
| 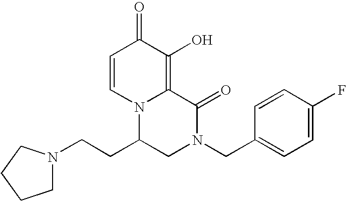 | 2-(4-fluorobenzyl)-9-hydroxy-4-(2-pyrrolidin-1-ylethyl)-3,4-dihydro-2H-pyrido[1,2-α]pyrazine-1,8-dione, TFA salt | 19 | 386 |
| 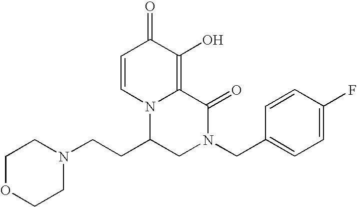 | 2-(4-fluorobenzyl)-9-hydroxy-4-(2-morpholin-4-ylethyl)-3,4-dihydro-2H-pyrido[1,2-α]pyrazine-1,8-dione, TFA salt | 19 | 402 |

While the foregoing specification teaches the principles of the present invention, with examples provided for the purpose of illustration, the practice of the invention encompasses all of the usual variations, adaptations and/or modifications that come within the scope of the following claims.

1. A compound of Formula Ia or Ib: 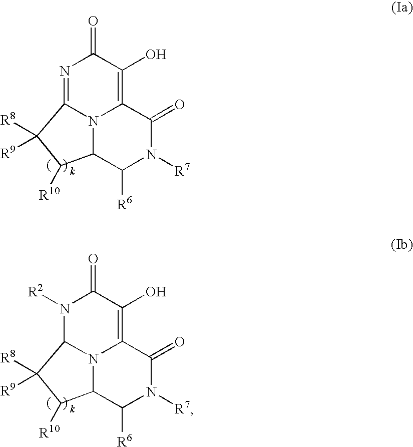
or a pharmaceutically acceptable salt thereof,
wherein k is an integer equal to 1 or 2;
R² is H or C₁₋₆ alkyl;
R⁶ is H or C₁₋₆ alkyl;
R⁷ is C₁₋₆ alkyl substituted with T, wherein T is:
(A) aryl or aryl fused to a 5- or 6-membered heteroaromatic ring containing from 1 to 4 heteroatoms independently selected from N, O and S, wherein the aryl or fused aryl is optionally substituted with from 1 to 5 substituents each of which is independently:
(1) —C₁₋₆ alkyl optionally substituted with —OH, —O—C₁₋₆ alkyl, —O—C₁₋₆ haloalkyl, —CN, —NO₂, —N(R^{a})R^{b}, —C(═O)N(R^{a})R^{b}, —C(═O)R^{a}, —CO₂R^{a}, —S(O)ₙR^{a} where n is an integer equal to zero or 1 or 2, —SO₂N(R^{a})R^{b}, —N(R^{a})C(═O)R^{b}, —N(R^{a})CO₂R^{b}, —N(R^{a})SO₂R^{b}, —N(R^{a})SO₂N(R^{a})R^{b}, —OC(═O)N(R^{a})R^{b}, or —N(R^{a})C(═O)N(R^{a})R^{b},
(2) —O—C₁₋₆ alkyl,
(3) —C₁₋₆ haloalkyl,
(4) —O—C₁₋₆ haloalkyl,
(5) —OH,
(6) halo,
(7) —CN,
(8) —NO₂,
(9) —N(R^{a})R^{b},
(10) —C(═O)N(R^{a})R^{b},
(11) —C(═O)R^{a},
(12) —CO₂R^{a},
(13) —SR^{a},
(14) —S(═O)R^{a},
(15) —SO₂R^{a},
(16) —SO₂N(R^{a})R^{b},
(17) —N(R^{a})SO₂R^{b},
(18) —N(R^{a})SO₂N(R^{a})R^{b},
(19) —N(R^{a})C(═O)R^{b},
(20) —N(R^{a})C(═O)—C(═O)N(R^{a})R^{b},
(21) —N(R^{a})CO₂R^{b},
(22) phenyl,
(23) benzyl,
(24) —HetB,
(25) —C(═O)—HetB, or
(26) —HetC, or
(B) a 5- or 6-membered heteroaromatic ring containing from 1 to 4 heteroatoms independently selected from N, O and S; wherein the heteroaromatic ring is
(i) optionally substituted with from 1 to 4 substituents each of which is independently halogen, —C₁₋₆ alkyl, —C₁₋₆ haloalkyl, —O—C₁₋₆ alkyl, —O—C₁₋₆ haloalkyl, or hydroxy; and
(ii) optionally substituted with 1 or 2 substituents each of which is independently aryl or —C₁₋₆ alkyl substituted with aryl;
R⁸ is:
(1) H,
(2) C₁₋₆ alkyl,
(3) N(R^{a})R^{b},
(4) N(R^{a})—CO₂R^{b},
(5) N(R^{a})—SO₂R^{b},
(6) N(R^{a})—C(═O)—R^{b},
(7) N(R^{a})—C(═O)—N(R^{a})R^{b},
(8) N(R^{a})—C(═O)—C(═O)—N(R^{a})R^{b},
(9) HetF,
(10) N(R^{a})—C(═O)—HetF, or
(11) N(R^{a})—C(═O)—C(═O)—HetF;
R⁹ is H, C₁₋₆ alkyl, or C₁₋₆ alkyl substituted with U, wherein U independently has the same definition as T;
each R¹⁰ is independently H or C₁₋₆ alkyl;
each HetB is independently a C₄₋₇ azacycloalkyl or a C₃₋₆ diazacycloalkyl, either of which is optionally substituted with from 1 to 4 substituents each of which is oxo or C₁₋₆ alkyl;
each HetC is independently a 5- or 6-membered heteroaromatic ring containing from 1 to 4 heteroatoms independently selected from N, O and S, wherein the heteroaromatic ring is optionally substituted with from 1 to 4 substituents each of which is independently halo, —C₁₋₆ alkyl, —C₁₋₆ haloalkyl, —O—C₁₋₆ alkyl, —O—C₁₋₆ haloalkyl, or hydroxy;
each HetF is independently a 4- to 7-membered saturated heterocyclic ring containing 1 or 2 N atoms, zero or 1 O atom, and zero or 1 S atom, wherein any ring S atom is optionally oxidized to SO or SO₂, and wherein the heterocyclic ring is attached to the rest of the compound via a N atom in the ring, and wherein the heterocyclic ring is optionally substituted with 1 or 2 substituents each of which is independently a —C₁₋₆ alkyl;
each aryl is independently phenyl or naphthyl;
each R^{a} is independently H or C₁₋₆ alkyl; and
each R^{b} is independently H or C₁₋₆ alkyl.

2. (canceled)

3. The compound according to claim 1, or a pharmaceutically acceptable salt thereof, wherein
R² is H or C₁₋₄ alkyl;
R⁶ is H or C₁₋₄ alkyl;
R⁷ is C₁₋₄ alkyl substituted with T, wherein T is phenyl, naphthyl, quinolinyl, or isoquinolinyl, wherein the phenyl, naphthyl, quinolinyl, or isoquinolinyl is optionally substituted with from 1 to 3 substituents each of which is independently halo, —C₁₋₄ alkyl, —O—C₁₋₄ alkyl, —C₁₋₄ fluoroalkyl, —SO₂—C₁₋₄ alkyl, —C(═O)—NH(—C₁₋₄ alkyl), —C(═O)—N(—C₁₋₄ alkyl)₂, or HetC;
R⁸ is:
(1) H,
(2) C₁₋₄ alkyl,
(3) N(R^{a})R^{b},
(4) N(R^{a})—CO₂R^{b},
(5) N(R^{a})—C(═O)—C(═O)—N(R^{a})R^{b},
(6) HetF, or
(7) N(R^{a})—C(═O)—C(═O)—HetF;
R⁹ is H, C₁₋₄ alkyl, or C₁₋₄ alkyl substituted with U, wherein U is phenyl, naphthyl, quinolinyl, or isoquinolinyl, wherein the phenyl, naphthyl, quinolinyl, or isoquinolinyl is optionally substituted with from 1 to 3 substituents each of which is independently halo, —C₁₋₄ alkyl, —O—C₁₋₄ alkyl, —C₁₋₄ fluoroalkyl, —SO₂—C₁₋₄ alkyl, —C(═O)—NH(—C₁₋₄ alkyl), —C(═O)—N(—C₁₋₄ alkyl)₂, or HetC;
each R¹⁰ is independently H or C₁₋₄ alky;
each HetC is independently a 5- or 6-membered heteroaromatic ring containing a total of 1 to 4 heteroatoms independently selected from 1 to 4 N atoms, zero or 1 O atom, and zero or 1 S atom, wherein the heteroaromatic ring is attached to the rest of the compound via a carbon atom in the ring, and wherein the heteroaromatic ring is optionally substituted with 1 or 2 substituents each of which is independently a —C₁₋₄ alkyl;
each HetF is independently a 5- or 6-membered saturated heterocyclic ring containing 1 or 2 N atoms, zero or 1 O atom, and zero or 1 S atom, wherein any ring S atom is optionally oxidized to SO or SO₂, and wherein the heterocyclic ring is attached to the rest of the compound via a N atom in the ring, and wherein the heterocyclic ring is optionally substituted with 1 or 2 substituents each of which is independently a —C₁₋₄ alkyl;
each R^{a} is independently H or C₁₋₄ alkyl; and
R^{b} is H or C₁₋₄ alkyl.

4. The compound according to claim 3, or a pharmaceutically acceptable salt thereof, which is a compound of Formula Ia1 or Ib1: 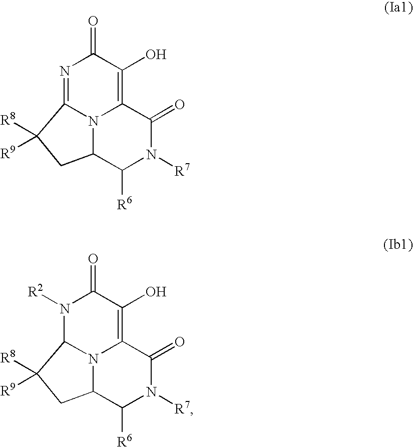
wherein
R² is H or C₁₋₃ alkyl;
R⁶ is H or C₁₋₃ alkyl;
R⁷ is CH₂-T, wherein T is phenyl which is optionally substituted with from 1 to 3 substituents each of which is independently halo, —C₁₋₃ alkyl, —O—C₁₋₃ alkyl, —C₁₋₃ fluoroalkyl, —SO₂—C₁₋₃ alkyl, —C(═O)—NH(—C₁₋₃ alkyl), —C(═O)—N(—C₁₋₃ alkyl)₂, or HetC;
R⁸ is:
(1) H,
(2) C₁₋₃ alkyl,
(3) N(R^{a})R^{b},
(4) N(R^{a})—C(═O)—O—C₁₋₄ alkyl,
(5) N(R^{a})—C(═O)—C(═O)—N(R^{a})R^{b},
(6) HetF, or
(7) N(R^{a})—C(═O)—C(═O)—HetF;
R⁹ is H, C₁₋₃ alkyl, or CH₂-U, wherein U is phenyl which is optionally substituted with from 1 to 3 substituents each of which is independently halo, —C₁₋₃ alkyl, —O—C₁₋₃ alkyl, —C₁₋₃ fluoroalkyl, —SO₂—C₁₋₃alkyl, —C(═O)—NH(—C₁₋₃ alkyl), —C(═O)—N(—C₁₋₃alkyl)₂, or HetC;
each R^{a} is independently H or C₁₋₃ alkyl; and
R^{b} is H or C₁₋₃ alkyl.

5. The compound according to claim 4, or a pharmaceutically acceptable salt thereof, wherein
R² is H or CH₃;
R⁶ is H or CH₃;
R⁷ is CH₂-T, wherein T is phenyl which is optionally substituted with from 1 to 3 substituents each of which is independently chloro, bromo, fluoro, CH₃, OCH₃, CF₃, SO₂CH₃, C(═O)NH(CH₃, C(═O)N(CH₃)₂, or oxadiazolyl;
R⁸ is:
(1) H,
(2) CH₃,
(3) N(H)CH₃,
(4) N(CH₃)₂,
(5) N(CH₃)—C(═O)—O—C₁₋₄ alkyl,
(6) N(CH₃)—C(═O)—C(═O)—N(H)CH₃,
(7) N(CH₃)—C(═O)—C(═O)—N(CH₃)₂,
(8) HetF, or
(9) N(CH₃)—C(═O)—C(═O)—HetF;
R⁹ is H, CH₃, or CH₂-U, wherein U is phenyl which is optionally substituted with from 1 to 3 substituents each of which is independently chloro, bromo, fluoro, CH₃, OCH₃, CF₃, SO₂CH₃, C(═O)NH(CH₃, C(═O)N(CH₃)₂, or oxadiazolyl; and
HetF is a heterocyclic ring selected from the group consisting of pyrrolidinyl, morpholinyl, thiomorpholinyl, piperidinyl, piperazinyl, and 4-methylpiperazinyl, wherein the heterocyclic ring is attached to the rest of the compound via a N atom in the ring.

6. (canceled)

7. (canceled)

8. (canceled)

9. A compound according to claim 1, or a pharmaceutically acceptable salt thereof, which is a compound selected from the group consisting of:
cis tert-butyl[7-(4-fluorobenzyl)-5-hydroxy-4,6-dioxo-2,4,6,7,8,8a-hexahydro-1H-3,7,8b-triazaacenaphthylen-2-yl]methylcarbamate;
trans tert-butyl[7-(4-fluorobenzyl)-5-hydroxy-4,6-dioxo-2,4,6,7,8,8a-hexahydro-1H-3,7,8b-triazaacenaphthylen-2-yl]methylcarbamate;
2,7-bis(4-fluorobenzyl)-5-hydroxy-2-(methylamino)-8,8a-dihydro-1H-3,7,8b-triazaacenaphthylene-4,6 (2H,7H)-dione;
cis 2-(dimethylamino)-7-(4-fluorobenzyl)-5-hydroxy-8,8a-dihydro-1H-3,7,8b-triazaacenaphthylene-4,6 (2H,7H)-dione;
cis N-[7-(4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide;
trans N-[7-(4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide; and
N-[7-(3-chloro-4-fluorobenzyl)-5-hydroxy-4,6-dioxo-2,4,6,7,8,8a-hexahydro-1H-3,7,8b-triazaacenaphthylen-2-yl]-N,N′,N′-trimethylethanediamide.

10. (canceled)

11. (canceled)

12. (canceled)

13. (canceled)

14. A compound according to claim 1, or a pharmaceutically acceptable salt thereof, wherein the compound is a compound of Formula VI: 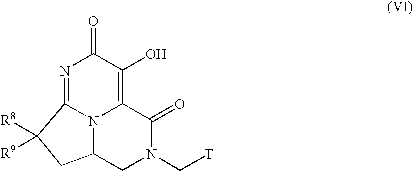
wherein
R⁸ is:
(1) H,
(2) C₁₋₃ alkyl,
(3) N(R^{a})R^{b},
(4) N(R^{a})—C(═O)—O—C₁₋₄ alkyl,
(5) N(R^{a})—C(═O)—C(═O)—N(R^{a})R^{b},
(6) HetF, or
(7) N(R^{a})—C(═O)—C(═O)—HetF;
R⁹ is H or CH₂-T;
T is 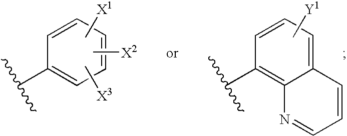
wherein X¹, X² and X³ are each independently selected from the group consisting of —H, halo, —C₁₋₄ alkyl, —O—C₁₋₄ alkyl, —C₁₋₄ fluoroalkyl, —SO₂—C₁₋₄ alkyl, —C(═O)—NH(—C₁₋₄ alkyl), —C(═O)—N(—C₁₋₄ alkyl)₂, and HetC;
Y¹ is —H, halo, —C₁₋₄ alkyl, or —C₁₋₄ fluoroalkyl;
each HetC is independently a 5- or 6-membered heteroaromatic ring containing from 1 to 4 heteroatoms independently selected from N, O and S, wherein the heteroaromatic ring is optionally substituted with 1 or 2 substituents each of which is independently a —C₁₋₃ alkyl;
HetF is a 5- or 6-membered saturated heterocyclic ring containing 1 or 2 N atoms, zero or 1 O atom, and zero or 1 S atom, wherein any ring S atom is optionally oxidized to SO or SO₂, and wherein the heterocyclic ring is attached to the rest of the compound via a N atom in the ring, and wherein the heterocyclic ring is optionally substituted with 1 or 2 substituents each of which is independently a —C₁₋₄ alkyl;
R^{a} is H or C₁₋₃ alkyl; and
R^{b} is H or C₁₋₃ alkyl.

15. A compound according to claim 14, or a pharmaceutically acceptable salt thereof, wherein R⁸ is:
(1) N(H)CH₃,
(2) N(CH₃)₂,
(3) N(CH₃)—C(═O)—O—C₁₋₄ alkyl,
(4) N(CH₃)—C(═O)—C(═O)—N(H)CH₃, or
(5) N(CH₃)—C(═O)—C(═O)—N(CH₃)₂,
(6) HetF, or
(7) N(CH₃)—C(═O)—C(═O)—HetF;
R⁹ is H or CH₂-T;
T is 4-fluorophenyl, 4-fluoro-3-methylphenyl, or 3-chloro-4-fluorophenyl; and
HetF is

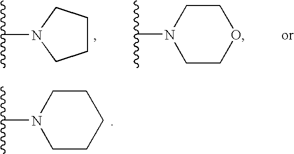

16. A pharmaceutical composition comprising an effective amount of a compound according to claim 1, or a pharmaceutically acceptable salt thereof, and a pharmaceutically acceptable carrier.

17. A method of inhibiting HIV integrase in a subject in need thereof which comprises administering to the subject an effective amount of the compound according claim 1, or a pharmaceutically acceptable salt thereof.

18. A method for treating infection by HIV or for treating or delaying the onset of AIDS in a subject in need thereof which comprises administering to the subject an effective amount of the compound according to claim 1, or a pharmaceutically acceptable salt thereof.

19. (canceled)

20. (canceled)

21. (canceled)

22. (canceled)

23. A pharmaceutical combination which is (i) a compound according to claim 1, or a pharmaceutically acceptable salt thereof, and (ii) an HIV infection/AIDS antiviral agent selected from the group consisting of HIV protease inhibitors, non-nucleoside HIV reverse transcriptase inhibitors and nucleoside HIV reverse transcriptase inhibitors; wherein the compound of (i) or its pharmaceutically acceptable salt and the HIV infection/AIDS antiviral agent of (ii) are each employed in an amount that renders the combination effective for inhibiting HIV integrase, for treating infection by HIV, or for treating or delaying the onset of AIDS.
